(19) United States
(12) Patent Application Publication
(10) Pub. No.: US 2015/0126969 A1
(43) Pub. Date: May 7, 2015

(54) DRUG DELIVERY DEVICE AND METHOD

(71) Applicant: Jianwei MO, Fremont, CA (US)

(72) Inventor: Jianwei MO, Fremont, CA (US)

(21) Appl. No.: 14/531,111

(22) Filed: Nov. 3, 2014

Related U.S. Application Data
(60) Provisional application No. 61/899,608, filed on Nov. 4, 2013.
(60) Provisional application No. 61/918,351, filed on Dec. 19, 2013.

(51) Int. Cl. A61M 5/315; A61M 5/19

(52) U.S. Cl. CPC A61M 5/31596; A61M 5/19
USPC 604/518; 604/89

(57) ABSTRACT

A method for combining a first liquid component with a second lyo component in a drug mixing apparatus is provided. The method comprises displacing a seal that prevents the first liquid component within a first barrel from coming into contact with the second lyo/liquid component within a second barrel. The method also comprises using an activating mechanism to move the first liquid component from the first barrel through at least one communication channel to an area within the second barrel holding the second lyo component, allowing the first liquid component to mix with the second lyo component to form a medicament. Additionally the method comprises displacing a second seal that prevents a medicament from entering an outlet channel. The method also comprises using the activating mechanism to eject the mixed medicament from the drug mixing apparatus through the outlet channel.

### CROSS REFERENCE TO RELATED APPLICATIONS

This application claims priority to U.S. Provisional Patent Application No. 61/899,608, filed Nov. 4, 2013, entitled “DRUG DELIVERY DEVICE AND METHOD,” and U.S. Provisional Patent Application No. 61/918,351, filed Dec. 19, 2013, entitled “DRUG DELIVERY DEVICE AND METHOD,” both of which are incorporated herein by reference for all purposes.

This application is related to U.S. patent application Ser. No. 13/463,464 filed May 3, 2012, entitled “MICRONEEDLE BASED TRANSDERMAL DRUG DELIVERY DEVICE AND METHOD” which is incorporated herein by reference for all purposes.

The present invention relates generally to needle-based drug delivery devices and methods that independently store components to be combined and then dispensed through a needle.

A large number of medical treatments involve the injection of a mixture/solution having two or more components. The mixtures/solutions may include one or more drugs, vaccines, diluents (e.g. a saline solution), liquids, etc. For many reasons, it is often beneficial to store the components separately and to only combine the components just before injection into a patient/recipient. In particular, storing some pharmacological components separately may increase the shelf-life of the medicaments (e.g., from days to years). Further, the efficacy/stability of mixed medicaments may considerably decrease over time. Accordingly, it is common practice for medical practitioners to combine medicament components relatively briefly before the administration of the medicament to a patient.

The current practice of mixing components has the potential for human error. For example, one method of mixing medicament components contemplates adding a liquid component, such as a diluent, to a lyophilized pharmacological component (lyo) stored in a sealed vial. Commonly this is accomplished by using a syringe to draw the diluent (such as a saline solution) into the syringe and thereafter eject the diluent from the syringe into a sealed vial that contains the lyo. After the lyo dissolves, the medicament mixture is drawn from the vial back into the syringe (or into a different syringe) and is thereafter injected into the patient. If too much or too little diluent is added, the injected dosage may be adversely affected. Further, since not all of the medicament mixture will be withdrawn from the vial after mixing, it is common practice to provide a greater amount of lyo drug component (e.g., 30% greater) in the vial than the actual prescribed dosage so as to ensure that the patient actually receives the prescribed dosage. Although this approach works well, some drugs are quite expensive and therefore the common practice of mixing a significantly greater amount of the pharmacological component than will actually be used can increase the expense of the injection.

These and other features of the present invention will be described in more details below in the detailed description of the invention and in conjunction with the following figures.

### BRIEF DESCRIPTION OF THE DRAWINGS

FIGS. 1A-1D illustrate a drug delivery device in accordance with an embodiment of the present invention.

FIGS. 2A-2E illustrate representative plunger and spring guard components suitable for use in accordance with the drug delivery device of FIGS. 1A-1D.

FIGS. 3A-3B illustrate spring activation components in accordance with an embodiment of the present invention.

FIGS. 4A-4B illustrate three-dimensional views of spring activation components in accordance with embodiments of FIGS. 3A-3B.

FIGS. 5A-5B illustrate a device having fill holes in accordance with an embodiment of the present invention.

FIGS. 6A-6B illustrate spring activation components in accordance with an embodiment of the present invention of FIGS. 5A-5B.

FIGS. 7A-7F illustrate another device in accordance with an embodiment of the present invention.

FIGS. 8A-8D illustrate a further device in accordance with an embodiment of the present invention.

FIGS. 9A-9D illustrate an additional device in accordance with an embodiment of the present invention.

FIGS. 10A-10D illustrate another device in accordance with embodiments of the present invention.

FIGS. 11A-11D illustrate components of a retractable needle apparatus in accordance with a device as provided in accordance with embodiments of the present invention.

FIGS. 12A-12F illustrate use of a retractable needle apparatus in accordance with a device as provided in accordance with the embodiment of FIGS. 11A-11D.

FIGS. 13A-13D illustrate components of a retractable needle apparatus in accordance with a device as provided in accordance with the embodiment of the present invention.

FIGS. 14A-14F illustrate use of a retractable needle apparatus in accordance with a device as provided in accordance with the embodiment of FIGS. 13A-13D.

FIGS. 15A-15D illustrate components of a retractable needle apparatus in accordance with a device as provided in accordance with the embodiment of FIGS. 3A-3D.

FIGS. 16A-16F illustrate use of a retractable needle apparatus in accordance with a device as provided in accordance with the embodiment of FIGS. 15A-15D.

FIGS. 17A-17D illustrate components of a retractable needle apparatus in accordance with a device as provided in embodiments of the present invention.

FIGS. 18A-18F illustrate use of a retractable needle apparatus in accordance with a device as provided in embodiments of the present invention.

FIG. 19 illustrates a 3-dimensional view of a base 1900 of a microneedle-based transdermal drug delivery device in accordance with embodiments of the present invention.

FIGS. 20A-20C illustrate a fully formed microneedle-based transdermal drug delivery device in accordance with embodiments of the present invention.

FIGS. 21A-21F illustrate use of a device with a retractable needle apparatus in accordance with embodiments of the present invention.

FIGS. 22A-22H illustrate a drug delivery device that contains separable, internally sealed chambers in accordance with embodiments of the present invention.

FIGS. 23A-23B illustrate illustrates a first casing uncoupled from a second casing of a device in accordance with embodiments of the present invention.

FIG. 24 illustrates a medicament component holder having angular connector plugs and recesses in accordance with embodiments of the present invention.

FIG. 25 illustrates a medicament component holder having t-shaped connector plugs and recesses in accordance with embodiments of the present invention.

FIGS. 26A-6B illustrate a drug delivery device with sealed chambers in accordance with embodiments of the present invention.

FIGS. 27A-27C illustrate a drug delivery device that has two outlets in accordance with embodiments of the present invention.

FIGS. 28A-28F illustrate a device having a retractable needle and internal seals in accordance with embodiments of the present invention.

### DETAILED DESCRIPTION OF THE PREFERRED EMBODIMENTS

The present invention will now be described in detail with reference to a few preferred embodiments thereof as illustrated in the accompanying drawings. In the following description, numerous specific details are set forth in order to provide a thorough understanding of the present invention. It will be apparent, however, to one skilled in the art, that the present invention may be practiced without some or all of these specific details. In other instances, well known process steps and/or structures have not been described in detail in order to not unnecessarily obscure the present invention.

In one aspect, a drug delivery device arranged to separately store two or more components of a medicament to be injected into a patient is provided. The medicament components are preferably stored in separate sealed compartments and mixed prior to injection. In other embodiments, the storage and mixing can be accomplished in an independent unit that may be attached to a drug delivery device such as a micro-needle, a syringe, etc. Additionally, corresponding methods for mixing liquid and/or solid components prior to injection of the mixture into a patient is provided. When used, the solid component may take the form of a lyophilized pharmacological component (lyo). A lyo has a solid form factor that can be dissolved in a diluent. The diluent may be a biologically inert solution such as saline, a liquid having a pharmacologically active ingredient, or any other suitable composition.

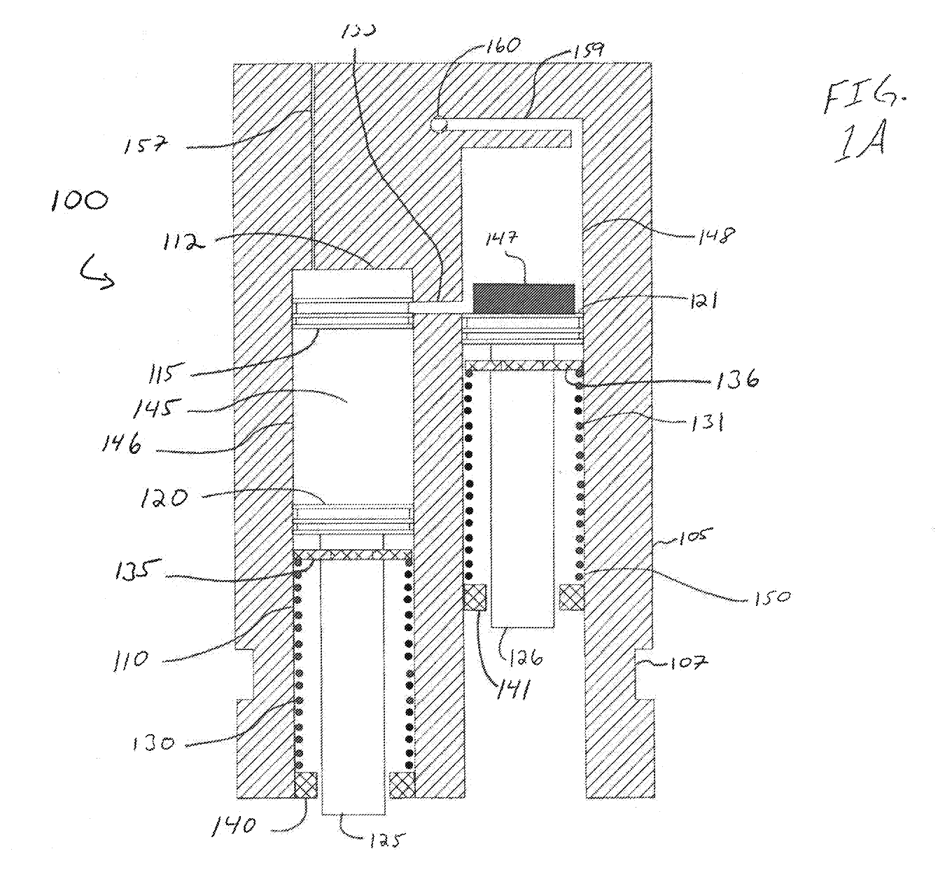

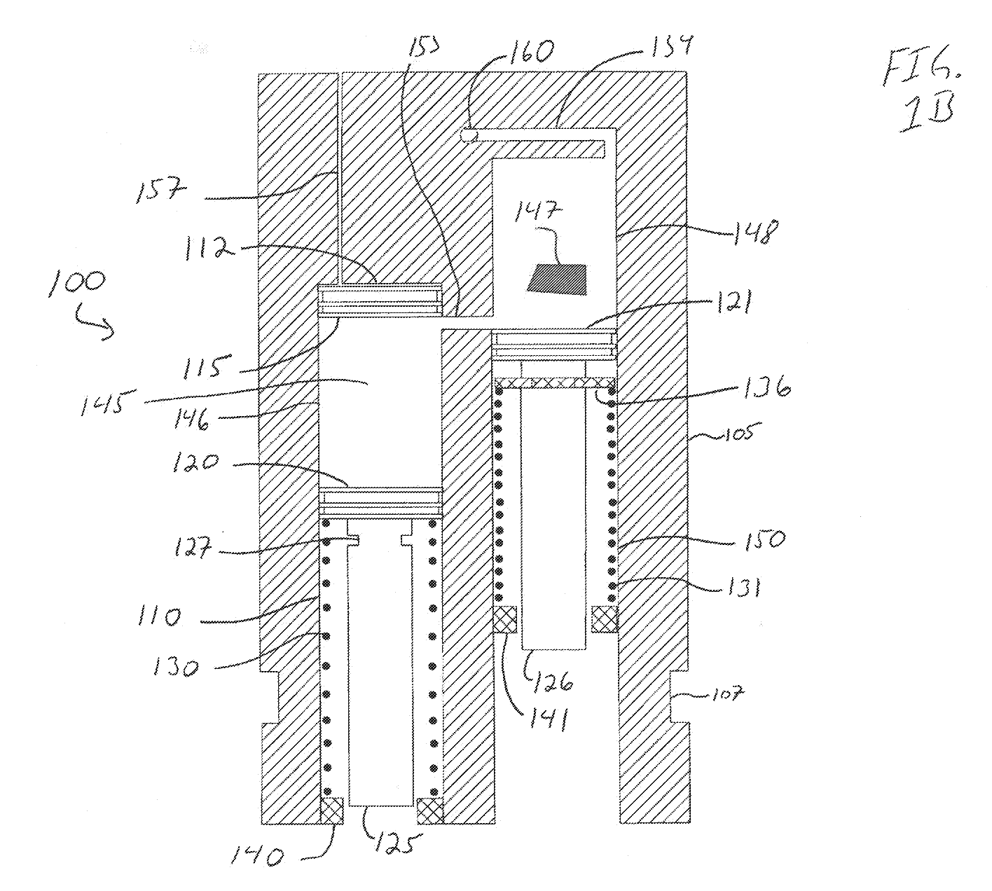

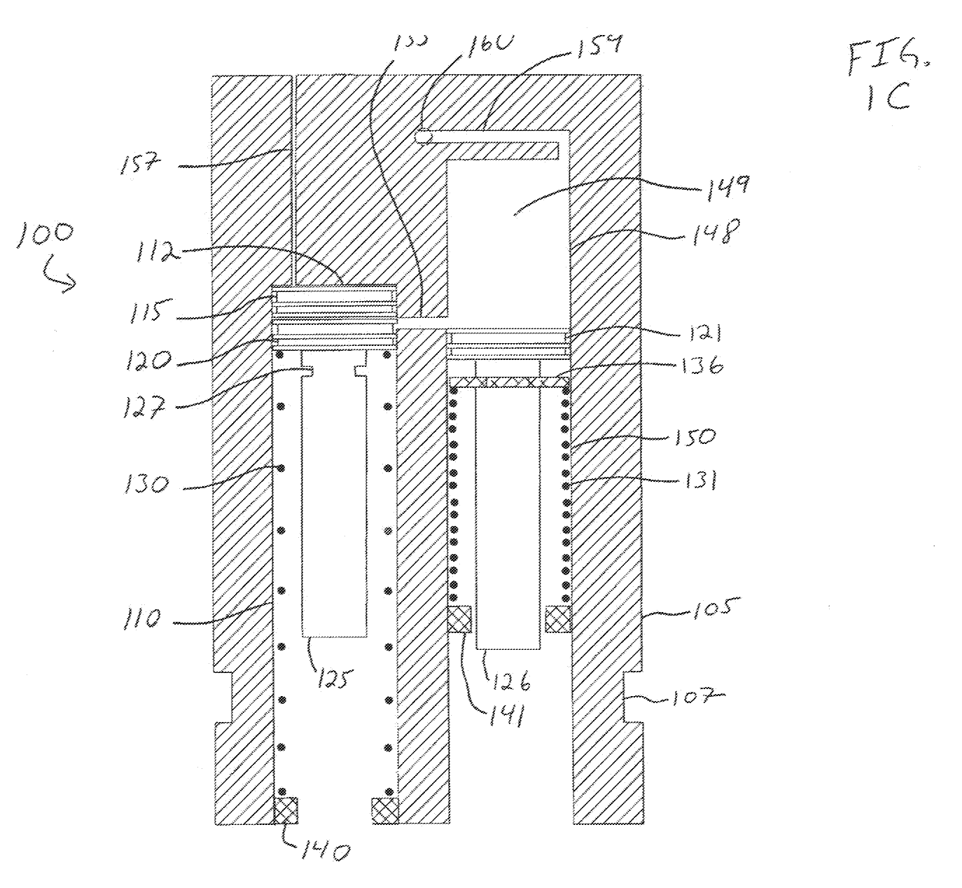

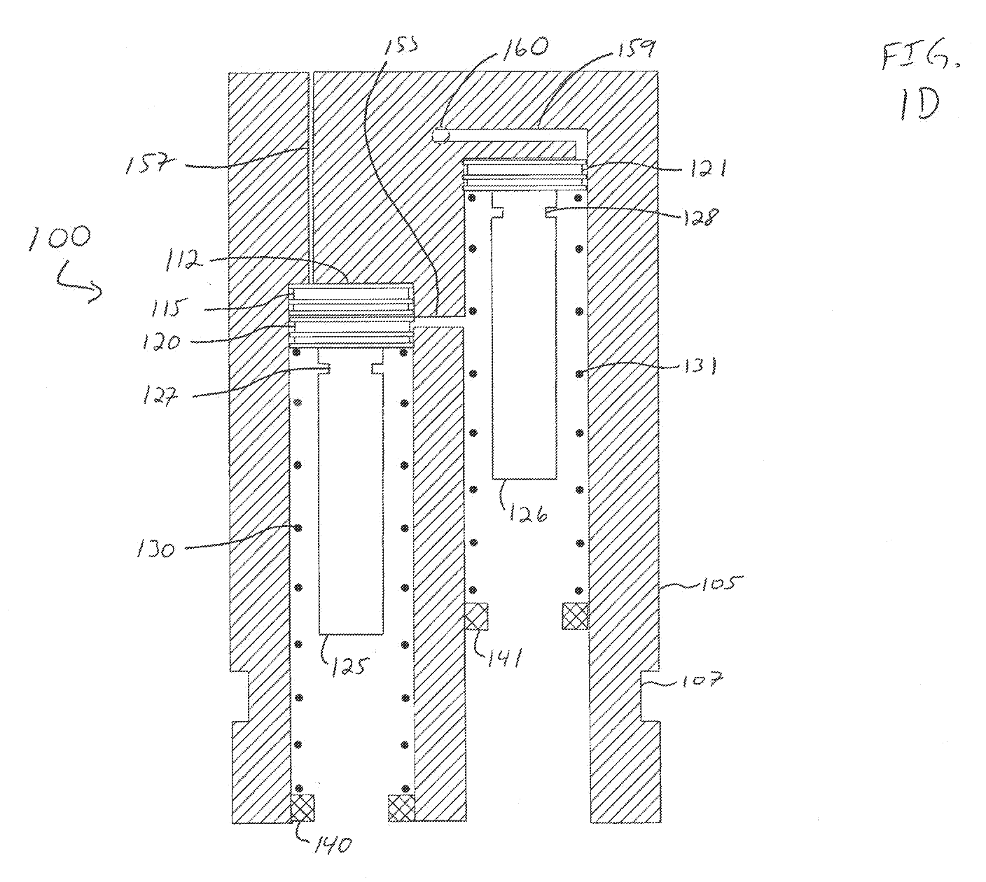

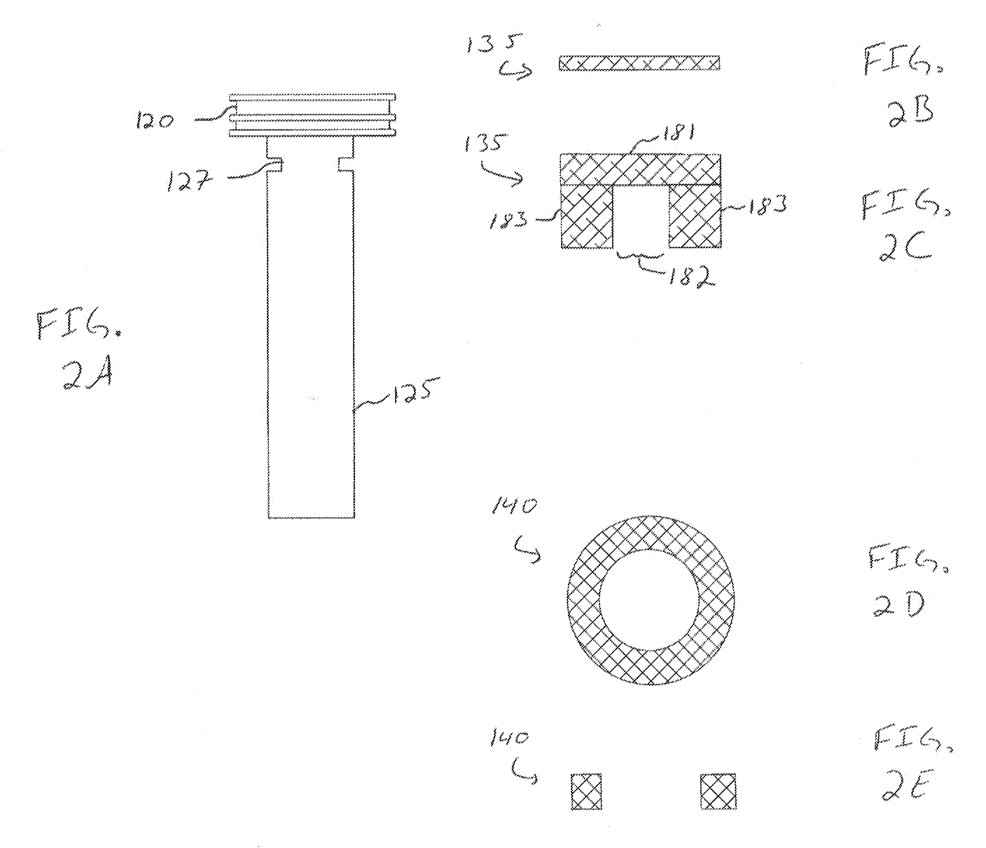

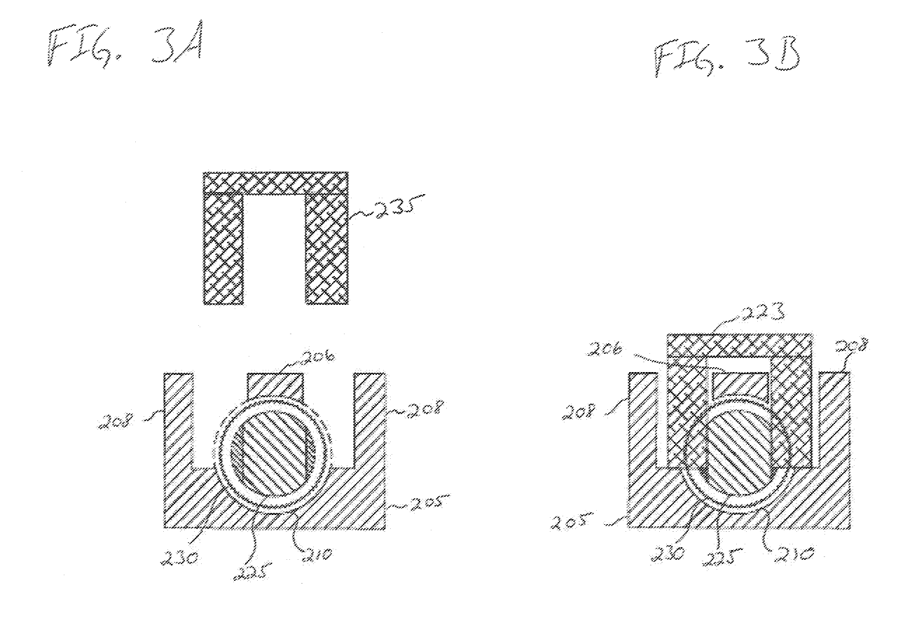

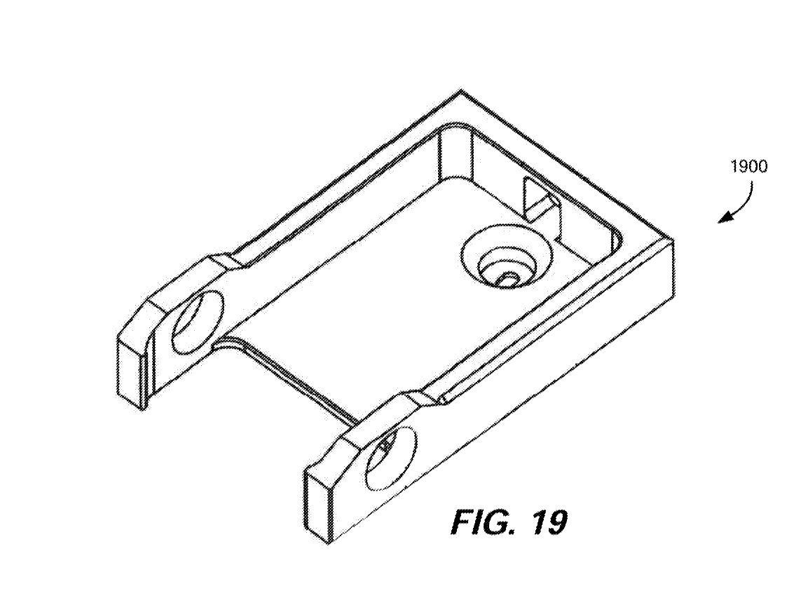

An example of a drug delivery device as described above is found in FIGS. 1A-1D. In particular, FIGS. 1A-1D illustrate a microneedle-based drug delivery device in accordance with a first embodiment 100 of the present invention. The device 100 has two barrels 110, 150 that each hold a component of a medicament. The two barrels 110, 150 are within an outer case 105. In the present embodiment, the outer case 105 has outer case notches 107 that allow the device 100 to be connected to a base of a microneedle-based transdermal drug delivery device as described in prior application U.S. patent application Ser. No. 13/463,464 filed May 3, 2012, entitled “MICRONEEDLE BASED TRANSDERMAL DRUG DELIVERY DEVICE AND METHOD,” which is incorporated herein by reference for all purposes. As an example, a suitable base for the microneedle-based transdermal drug delivery device is illustrated in FIG. 3A of the above-referenced application, which is reproduced at FIG. 19A of the current application. Additionally, an example of the base connected to a drug delivery device as provided herein in FIGS. 1A, 1B, and 2A of the above-referenced application, which are reproduced as FIGS. 19B-19D of the current application.

A first barrel 110 contains a liquid component 145 of a medicament. As seen in FIGS. 1A-1D, first barrel 110 contains a stopper 115 and a plunger 125 that carries a plunger seal 120. In the illustrated embodiment, a compressed spring 130 is used to push the plunger distally within first barrel 110 as generally described in the incorporated patent application. The spring 130 is held in place between spring guard base 140 and a removable spring guard cap 135. The plunger 125 may also include notches 127 (see FIG. 1B) suitable for engaging the spring guard cap 135 as will be discussed in more detail below. A liquid storage chamber 146 is effectively formed between stopper 115 and plunger seal 120. The liquid storage chamber 146 holds a liquid component 145 of a medicament mixture intended for injection.

Device 100 also includes a communication channel 155 that connects first barrel 110 to second barrel 150. Much like the first barrel 110, the second barrel 150 has a second plunger 126 that carries a second plunger seal 121 and a second spring 131 arranged to actuate the second plunger 126. Second plunger 126 may also include notches 128 (see FIG. 1D). Similarly to the first spring 130, the second spring 131 may be held in place between a removable spring guard cap 136 and spring guard base 141. The region of the second barrel 150 distal of the plunger seal 121 effectively forms a mixing chamber 148. The mixing chamber 148 is arranged to store a solid component 147 (which in this example may take the form of a lyo). A delivery channel 159 connects the mixing chamber 146 to an outlet 160 which may feed a micro-needle (as described in the incorporated patent application) or other delivery mechanism.

During use, the first plunger 125 may be actuated to deliver the liquid component 145 from the liquid storage chamber 146 to the mixing chamber 148 as best seen in FIG. 1C. Within the mixing chamber 148, the liquid component 145 dissolves the lyo component 147 to form a medicament mixture/solution 149 for injection. After the lyo component 147 has dissolved, the medicament mixture 149 may be injected by actuating the second plunger 126.

More particularly, when the plunger 125 is in a first (loaded) position as illustrated in FIG. 1A, the spring guard cap 135 prevents the plunger 125 from moving distally and the stopper 115 is positioned such that it seals communication channel 155 thereby preventing the liquid component 145 from leaking into the mixing chamber 148 in the second barrel 150. When spring guard cap 135 is removed, the compressed spring 130 is released such that the spring pushes plunger 125 and plunger seal 120 distally within the first barrel 110. The liquid 145 is typically substantially incompressible and therefore pushes stopper 115 distally as well to a position where communication channel 155 is opened, allowing fluid communication between liquid storage chamber 146 and the second barrel 150. In the illustrated embodiment, communication channel 155 is proximally far enough from the distal end 112 of first barrel 110 so as to allow stopper 115 to move past communication channel 155 and clear the opening of communication channel 155, thereby allowing liquid component 145 to flow into communication channel 155. This second position is diagrammatically illustrated in FIG. 1B which shows the first plunger 125 part way through its stroke with the stopper 115 moved to open the communication channel 155. In this second position, liquid component 145 is able to pass from first barrel 110 to second barrel 150 through communication channel 155. The air that previously resided in the space between stopper 115 and the distal end of first barrel 110 is vented through vent 157.

As the plunger 125 and plunger seal 120 continue movement distally within the first barrel 110, the liquid 145 follows the path of least resistance and is thereby forced into the mixing chamber 148 of the second barrel until the plunger 125 comes to rest and/or the plunger seal 120 seals off communication channel 155. In the illustrated embodiment (see FIG. 1C), distal movement of the plunger 125 is stopped when plunger seal 120 comes into contact with stopper 115 and distal movement of the stopper 115 is physically constrained by end wall 112 of barrel 110. In the illustrated embodiment, the distance between the end of communication channel 155 and the barrel end wall 112 is selected to substantially match the length of the stopper 115 so that the plunger seal comes to rest adjacent the communication channel 155. This has the advantage of helping ensure that substantially all of the liquid component 145 is ejected from the first barrel 110 which gives very good control over the amount of liquid that is delivered to the mixing chamber 148 of the second barrel. However, it should be appreciated that in other embodiments, the distance between the end of the communication channel 155 and the barrel end wall 112 may vary widely.

When liquid component 145 passes through communication channel 155 and into the mixing chamber 148, liquid component 145 interacts with solid component 147 (which in this example takes the form of a lyo). In particular, lyo component 147 dissolves within liquid component 145. This is diagrammatically illustrated in FIG. 1B which shows the first plunger 125 part way through its stroke. At this point, some (but not all) of the liquid component 145 has been transferred to the mixing chamber 148 and has begun dissolving lyo 147 as diagrammatically illustrated.

FIG. 1C illustrates the drug delivery device 100 in a stage where the first plunger 125 has been fully actuated and the second plunger 126 remains unactuated. In this stage, the liquid component 145 has been transferred to mixing chamber 148 where it completely dissolves the lyo component 147. Once the lyo 147 has fully dissolved, the resulting medicament mixture/solution 149 is ready for injection. The resulting mixture/solution may then be ejected from the assembly and administered to the patient as a medical treatment by actuating the second plunger 126.

The second plunger 126 is actuated by removing the second spring guard cap 136. When spring guard cap 136 is removed, spring 131 is released which pushes plunger 126 distally to deliver the mixture to a micro-needle (not shown) through delivery channel 159 and outlet 160. FIG. 1D shows the drug delivery device in a stage after completing actuation of the second plunger 126, having transferred the mixture/solution 149 through outlet 160. As can readily be seen in FIGS. 1C and 1D, after the first plunger 125 has been fully actuated, the first plunger seal 125 blocks the communication channel 155. This prevents the mixture from flowing back into the first barrel 110 during actuation of the second plunger 126.

FIGS. 2A-2E illustrate representative plunger and spring guard components suitable for use with the embodiment of FIGS. 1A-1D. In particular, FIG. 2A illustrates plunger 125 having plunger notches 127 and plunger seal 120. FIGS. 2B and 2C are respectively top and cross-sectional views of spring guard cap 135. The spring guard cap 135 is substantially U-shaped with a top section 181 and two legs 183 that are separated by a gap 182. The spacing of the legs is arranged to correspond to the width of the plunger 125 between the plunger notches 127 so spring guard cap 135 can be inserted into a slot (not shown) in the top surface of device 100 with the legs 183 straddling the narrowed section of the plunger 125 between the notches 127. In this position, the top section 181 of the spring guard cap 135 projects above the plunger 125 through a slot type opening in the top surface of the device housing as will be described in more detail below. The spring 130 is positioned to engage and therefore push against the spring guard cap 135 (i.e., against the proximal side of legs 183 and/or top section 181). The distal wall of the slot in the housing engages the top section 181 of spring guard cap 135 to prevent the spring guard cap 135 from moving distally thereby holding spring guard cap 135 in place against the force of spring 130. The top section 181 extends above the device housing so that it can be grasped and pulled free by a user. Removing the spring guard cap 135 releases spring 130 to actuate the plunger 125. Although a specific spring guard cap geometry is described, it should be appreciated that a wide variety of different geometries and structures can be used to hold and release the spring 130. FIGS. 2D and 2E are front and cross-sectional views of a suitable spring guard base 140. In the illustrated embodiment, a washer type structure is used, although it should be appreciated that a wide variety of other structures can be used in other embodiments.

An illustration of spring activation components similar to those described in FIGS. 1A-2E is provided in FIGS. 3A-3B. FIGS. 3A-3B show diagrammatic cross-sectional views of a barrel 210 in outer case 205. Barrel 210 is similar to barrel 110. In FIG. 3A, a spring guard cap 235 is shown prior to its insertion into barrel 210, while FIG. 3B shows the spring guard cap 235 after it has been inserted into barrel 210. As seen in FIG. 3A, spring guard cap 235 is substantially U-shaped, with a top portion and two leg portions coming down from the top portion. These leg portions of spring guard cap 235 are compatible with the slots formed between side portions 208 and bridge portion 206. These slots are in line with the plunger notches of plunger 225. The plunger notches as shown in FIGS. 3A-3B are similar to the plunger notches 127 discussed above with respect to FIGS. 1A-1D and 2A-2E. As such, spring guard cap 235 is able to fit within the outer case and block a portion of barrel 210 that overlaps with spring 230. This, in turn, allows spring guard cap 235 to block spring 230 from expanding (i.e. becoming uncompressed). Accordingly, in FIG. 3B, spring guard cap 235 is placed within barrel 210 so as to prevent compressed spring 230 from expanding.

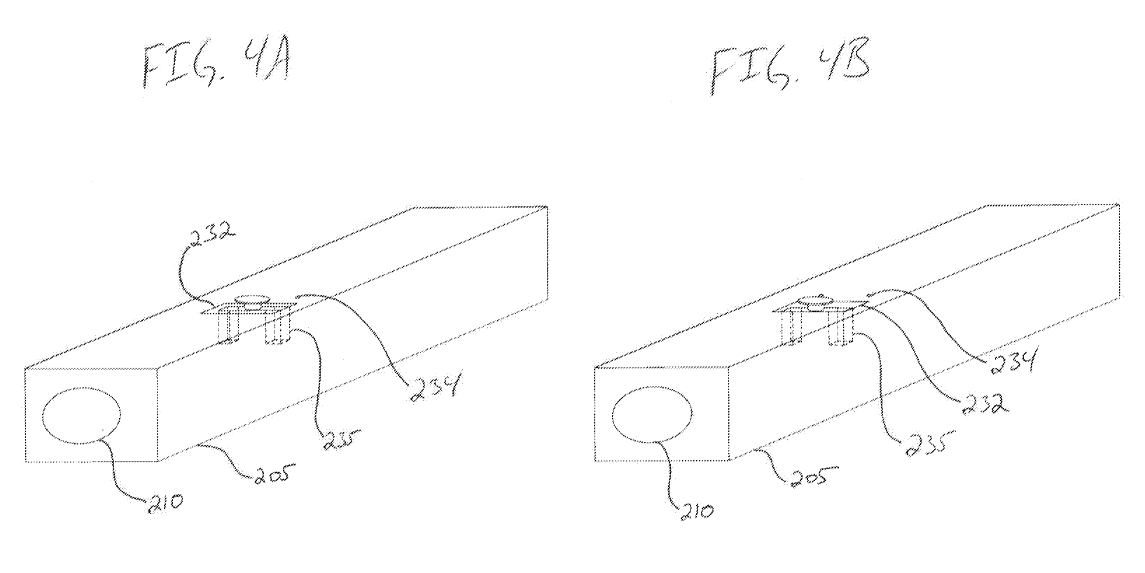

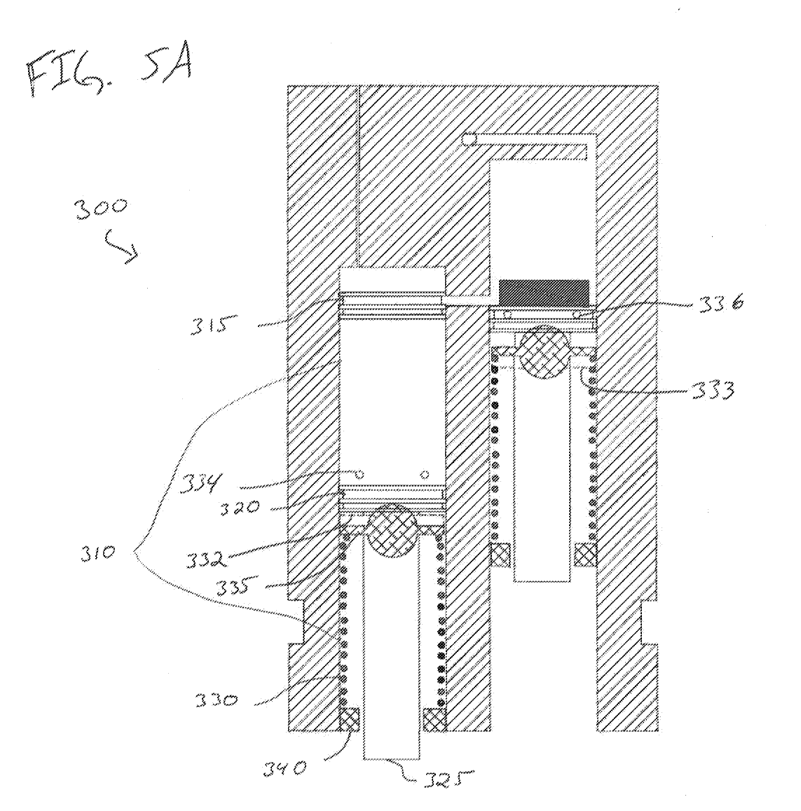

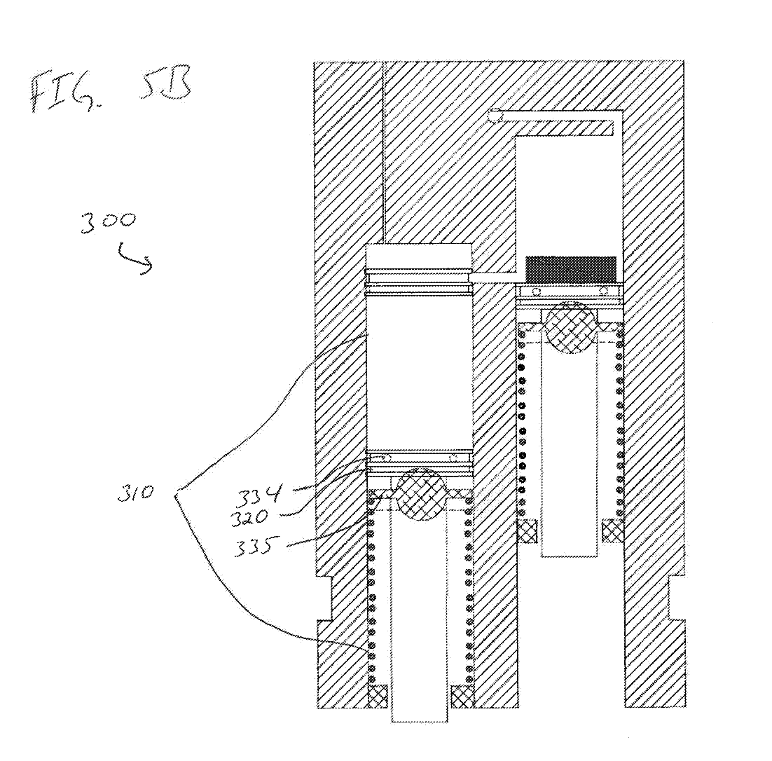

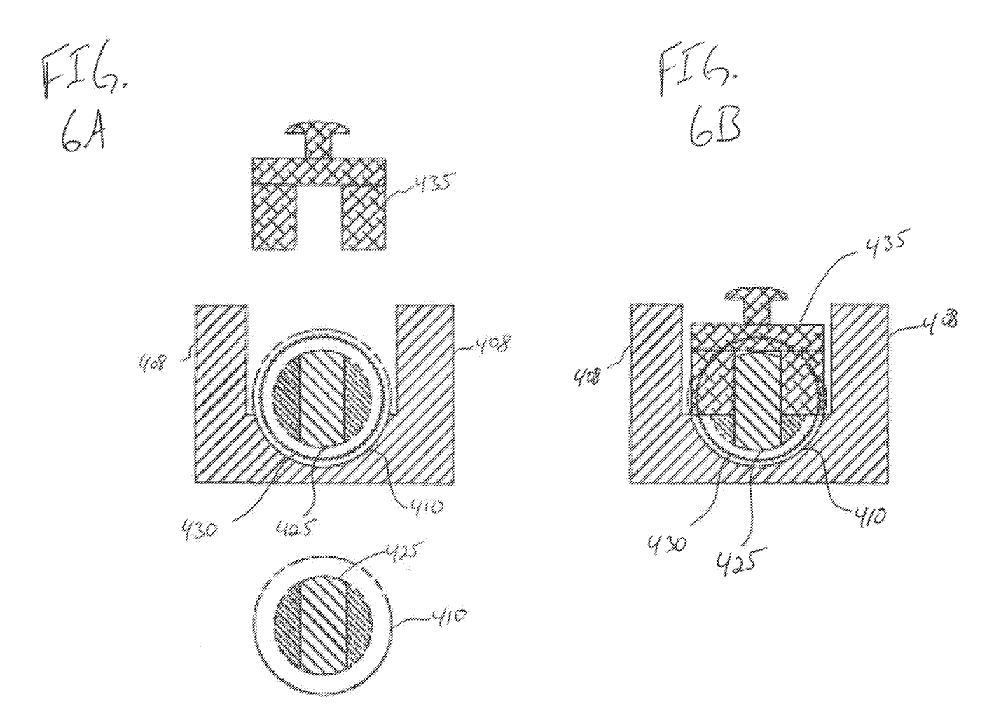

Three-dimensional views of spring guard cap 235 within barrel 210 are seen in FIGS. 4A-4B. FIG. 4A illustrates outer case 205, barrel 210, channels 232, fill holes 234, and spring guard cap 235. Spring guard cap 235 is able to move across channels 232. As seen in FIG. 4A, spring guard cap 235 is in a first position that is at the top of channels 232. The use of fill holes 234 is described with regard to FIGS. 5A and 5B. In FIG. 4B, a second position is illustrated where spring guard component 235 is at the bottom of channels 232.

FIGS. 5A-5B illustrate a device in accordance with an embodiment 300 of the present invention. Device 300 has two barrels used to mix a liquid component and a lyo component, and device 300 is the same as device 100 except that device 300 also has channels 332, 333 and fill holes 334, 336. As seen in FIG. 5A, first barrel 310 has a stopper 315 and a plunger seal 320 within first barrel 310, where plunger seal 320 is movable using plunger 325 based on a spring 330 that is compressed within first barrel 310. In particular, plunger 325 has plunger notches that are able to hold spring guard cap 335 so as to keep spring 330 from extending. Further, spring guard base 340 similarly keeps spring 330 from extending. Additionally, spring guard cap 335 is itself movable across the length of channel 332.

Channel 332 matches or exceeds the width of spring guard cap 335 and has a length that is greater than the length of spring guard cap 335. In this way, spring guard cap 335 is able to move proximately along the length of channel 332 so as to further compress spring 330. Additionally, spring guard cap 335 has a nob at the top of spring guard cap 335 so as to allow spring guard cap 335 to be pulled back to compress spring 330. The further compression of spring 330 allows plunger seal 325 to be drawn back so as to expose fill holes 334. This is seen in FIG. 5A. Fill holes 334 are used to fill a portion of first barrel 310 with liquid in a way that control accurate dosage (volume) and minimizes and/or eliminates the addition of air in the portion of first barrel 310 between plunger seal 325 and stopper 315.

Once the liquid portion of first barrel 310 has been filled using fill holes 334, spring guard cap 335 may be moved into a position so as to push plunger seal 320 to cover fill holes 334. This view is seen in FIG. 5B. From this position, device 300 is able to be used to mix and dispense a medicament in the same way that is described above in FIGS. 1A-1D.

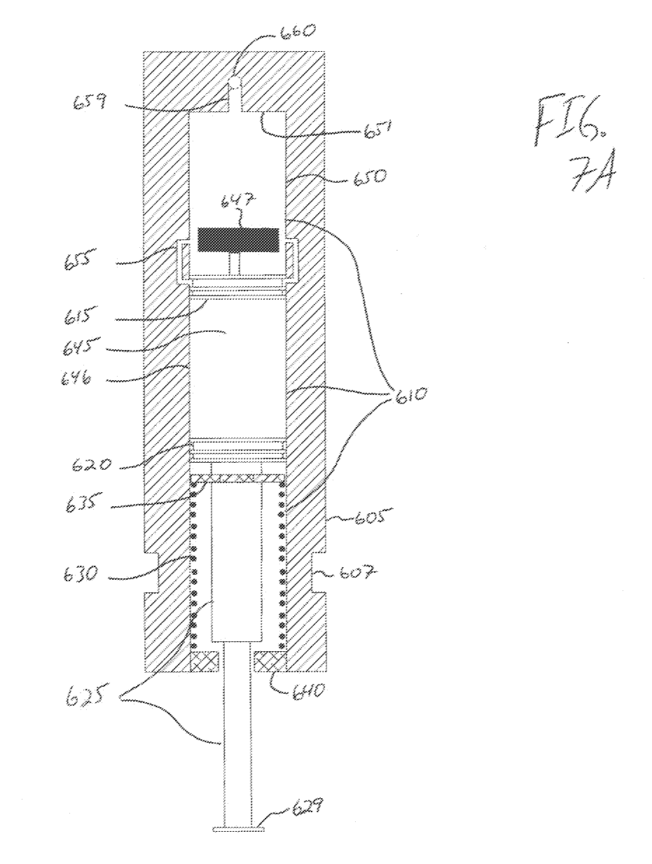

In the same way that FIGS. 3A-3B illustrated spring activation components similar to those described in FIGS. 1A-2E, FIGS. 6A-6B illustrates spring activation components similar to those described in FIGS. 5A-5B. The spring activation components of FIGS. 6A-6B are the same as the spring activation components of FIGS. 3A-3B except FIGS. 6A-6B do not have a bridge similar to bridge 206 and the spring guard cap 435 of FIGS. 6A-6B has a tab.

Similar to FIGS. 3A-3B, FIGS. 6A-6B show a cross-sectional view of a barrel 410 in outer case 405. Barrel 410 is similar to barrel 310. In FIG. 3A, a spring guard cap 435 is shown prior to its insertion into barrel 410, while FIG. 6B shows the spring guard cap 435 after it has been inserted into barrel 410. As seen in FIG. 6A, spring guard cap 435 is substantially U-shaped, with a top portion and two leg portions coming down from the top portion. These leg portions of spring guard cap 435 are compatible with the slots formed between side portions 408. These slots are in line with the plunger notches of plunger 225. Further, since barrel 410 doesn't have a bridge to prop up spring guard cap 435, the spring guard cap sits relatively lower in barrel 410 compared to spring guard cap 235 of FIGS. 3A-3B. As such, spring guard cap 435 has a tab that extends beyond the diameter of barrel 410. In this way, spring guard cap 435 is able to fit within the outer case and block a portion of barrel 410 that overlaps with spring 430. This, in turn, allows spring guard cap 435 to block spring 430 from expanding (i.e. becoming uncompressed). Accordingly, in FIG. 6B, spring guard cap 435 is placed within barrel 410 so as to prevent compressed spring 430 from expanding. Additionally, the spring guard cap 435 of FIGS. 6A-6B is able to increase or decrease compression of spring 430. In particular, spring guard cap 435 is able to compress spring 330 so as to move plunger 425 to reveal or cover fill holes 434 as discussed above in FIGS. 5A-5B.

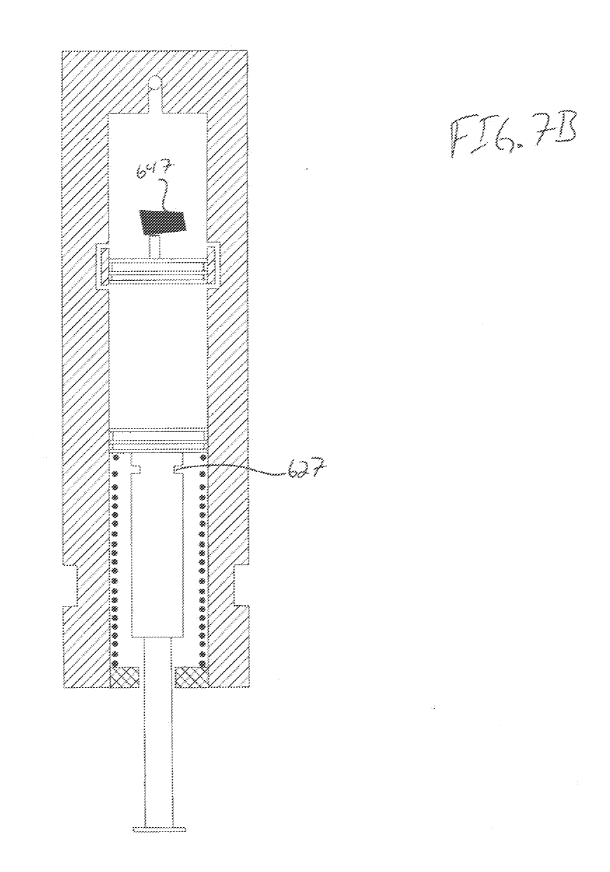

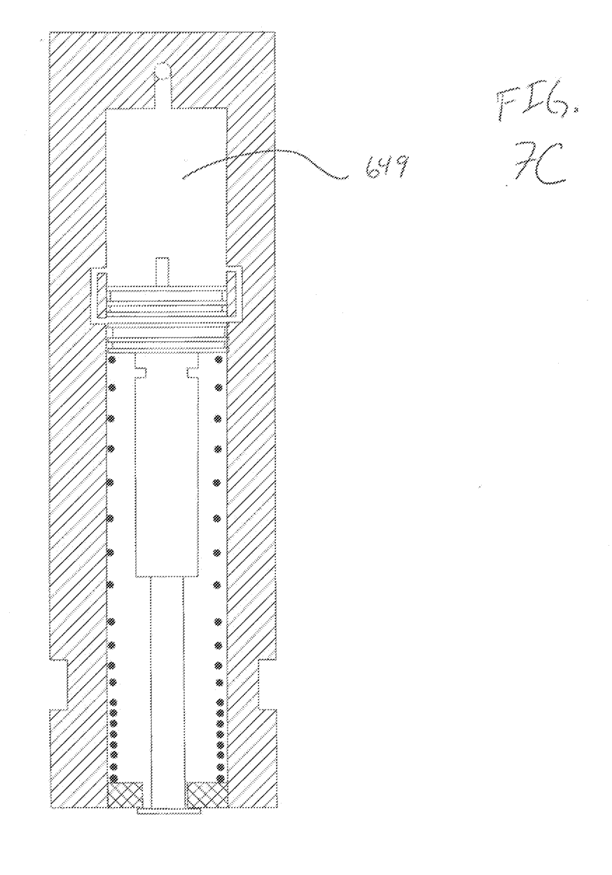

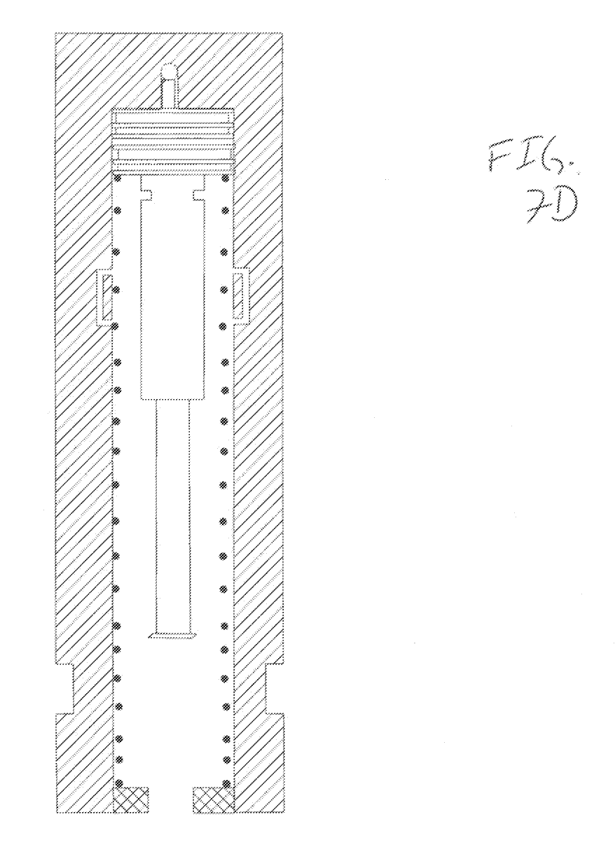

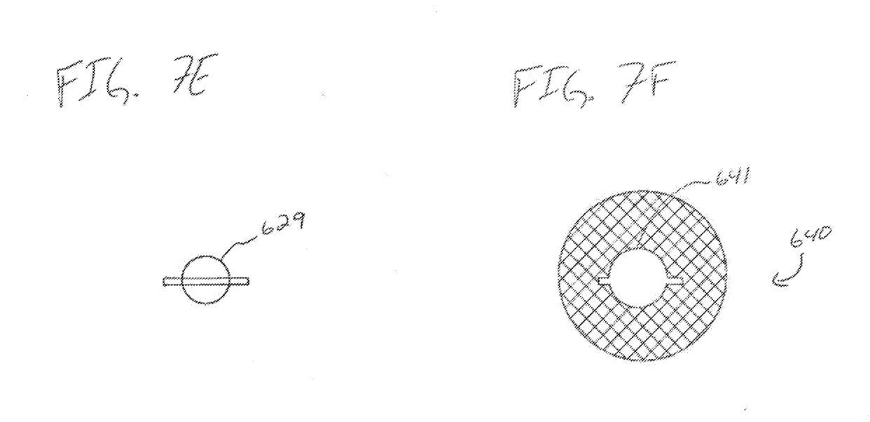

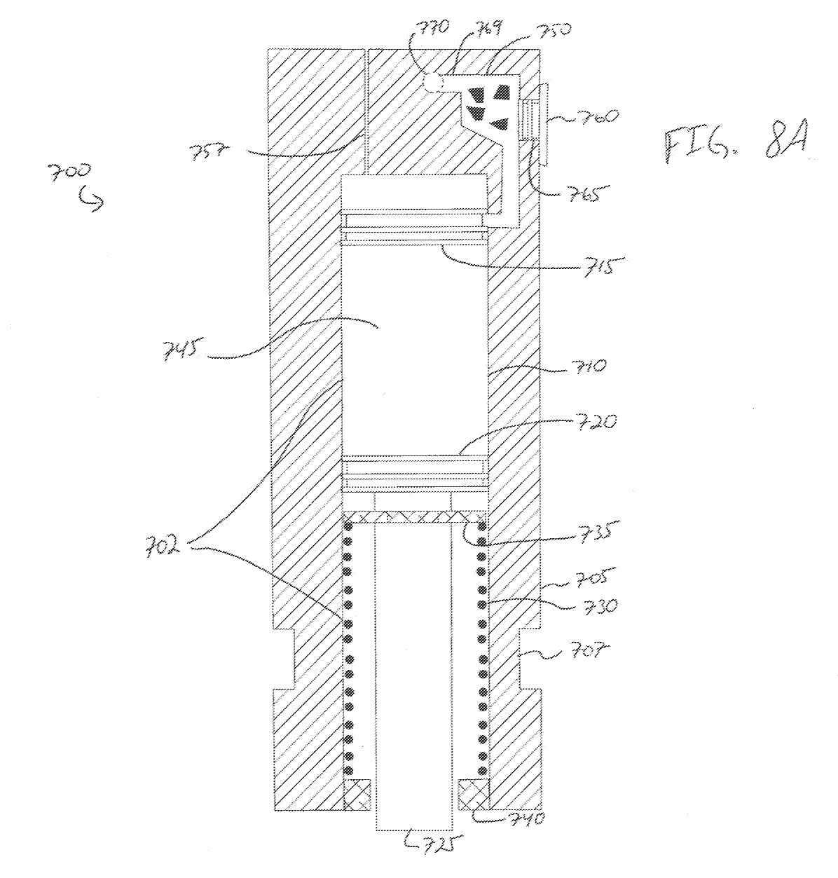

Referring next to FIGS. 7A-7F, another embodiment 600 of a drug delivery device that incorporates a single barrel will be described. In contrast to the two-barrel drug delivery device seen in devices 100 and 300, device 600 has a single barrel 610 that has serially arranged liquid and solid holding chambers. As shown in FIGS. 7A-7F, device 600 is arranged to mix a liquid component and a solid/lyo component prior to injection.

Barrel 610 includes a liquid chamber 646 and a mixing chamber 650. The liquid chamber 646 is arranged to hold a liquid component 645 and is defined by the portion of the barrel 610 that is in between stopper 615 and plunger seal 620. The mixing chamber 650 is arranged to hold a solid component 647 and is defined by the portion of the barrel between the stopper 615 and the distal end 651 of the barrel 610. When the plunger 625 is initially activated liquid component 645 is able to pass from the liquid chamber 646 to mixing chamber 650 via side channels 655. FIG. 7A illustrates the configuration when device 600 is in a locked and loaded position. In this first stage, side channels 655 are plugged by stopper 615 such that the liquid chamber is isolated from the mixing chamber by the stopper which acts as a seal. The plunger 625 is prevented from moving distally by spring guard cap 635. When a user is ready to mix the medicament components, the spring guard cap 635 is lifted/removed thereby releasing spring 630 which pushes the plunger 625 distally. Since the liquid 645 is substantially incompressible, distal movement of the plunger moves stopper 615 forward as well until the position represented in FIG. 7B is reached. At this point, side channels 655 are opened thereby allowing liquid component 645 to pass around the stopper 615 to the mixing chamber 650. Friction between the stopper 615 and the barrel walls adjacent the channels 655 causes the stopper 615 to stop moving distally at this point as the liquid 645, taking the path of least resistance, passes through the side channels 655 into the mixing chamber 650 as the plunger 625 continues to move distally. The liquid component 645 entering the mixing chamber 650 begins interacting with (e.g. dissolving) the lyo 647 as also diagrammatically illustrated in FIG. 7B.

The plunger 625 continues to translate distally under the influence of spring 630 until it reaches the position illustrated in FIG. 7C where plunger seal 620 engages the stopper 615 and plunger base 629 comes into contact with the spring guard base 640 which prevents further distal movement of the plunger 625. This causes all of the liquid to be ejected from the liquid chamber 646 and transferred to the mixing chamber 650 apart from a small residual amount found in the side channels 655. With the liquid 645 transferred to mixing chamber 650 as shown in FIG. 7C, the transferred liquid 645 dissolves the lyo 647 resulting in a medicament mixture/solution 649. The length of the side channels 655 are preferably long enough such that the liquid component 645 can flow around the stopper 615 during the mixing step, but short enough so that fluid cannot bypass the stopper/plunger seal combination during injection. Once liquid 645 dissolves lyo 647, the resultant medicament 649 is ready for injection into a patient.

At this stage, both the device 600 and the medicament mixture 649 are ready for injection. The plunger 625 can then be manually actuated by releasing the plunger base 629 from the spring guard base 640. Once the plunger top 629 is released, both the plunger seal 620 and the stopper 615 move distally under the influence of spring 630 pushing the mixture 649 through delivery channel 659 and outlet 660 which may be coupled to a micro-needle or other suitable delivery device as previously described. For example, outlet 660 may feed a syringe needle which may have at least one side opening communicating with the hollow interior of the syringe needle (e.g., similar to the side openings as described in the incorporated patent application). When the stopper 615 engages the end wall 651 of barrel 610, the injection is complete.

FIGS. 7E and 7F illustrate plunger base component 629, which has side extensions, or “wings,” that allow plunger base component 629 to lock or pass through spring guard base component 640 as seen in FIG. 7F. Spring guard base component 640 is the same as spring guard base component 140 except that spring guard base component 640 has a keyhole 641 that matches the shape of plunger base component 629.

The shapes of plunger base component 629 and keyhole 641 allows device 600 to have a two-step plunger activation mechanism. In a first position, plunger base 629 is oriented such that the wings of plunger base 629 are perpendicular to the wing-sized openings of keyhole 641. When the spring guard cap is released, the plunger 625 is able to move distally along barrel 610. However, since the wings of plunger base 629 are oriented in a position that is perpendicular to keyhole 641, plunger base 629 is only able to distally move plunger 625 until plunger base 629 presses against spring guard base 640. Once plunger base 629 has reached spring guard base 640, the plunger 625 may be manually rotated until the wings of plunger base 629 are aligned with the wing-sized openings of keyhole 641. As such, in the second position, plunger base 629 can pass through keyhole 641 of spring guard base 640. Accordingly, plunger 625 may advance so as to expel the medicament from device 600 by further advancing distally along barrel 610.

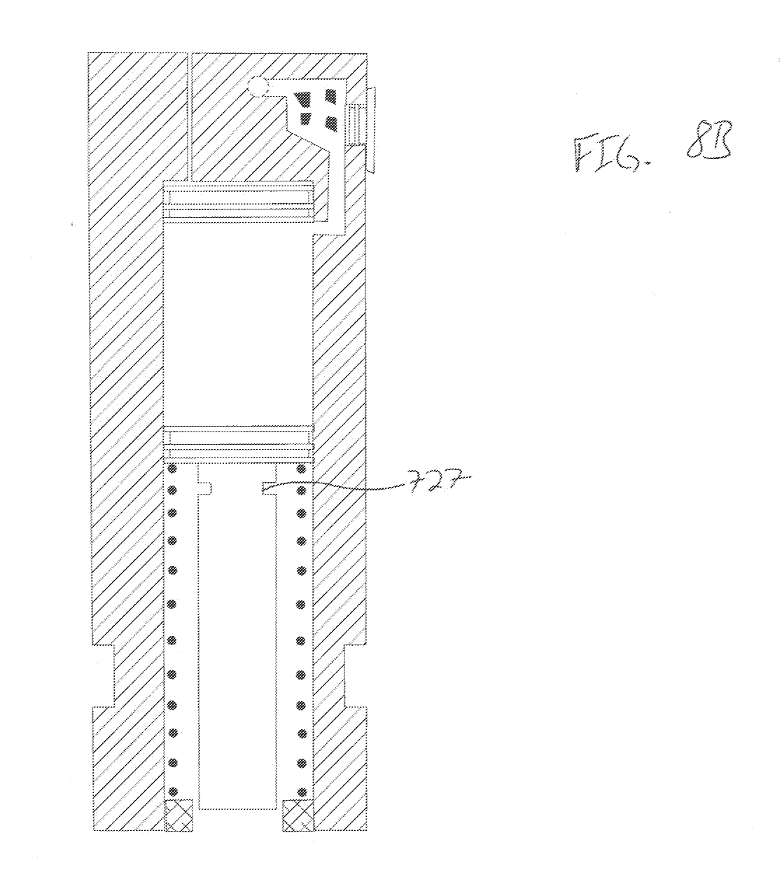

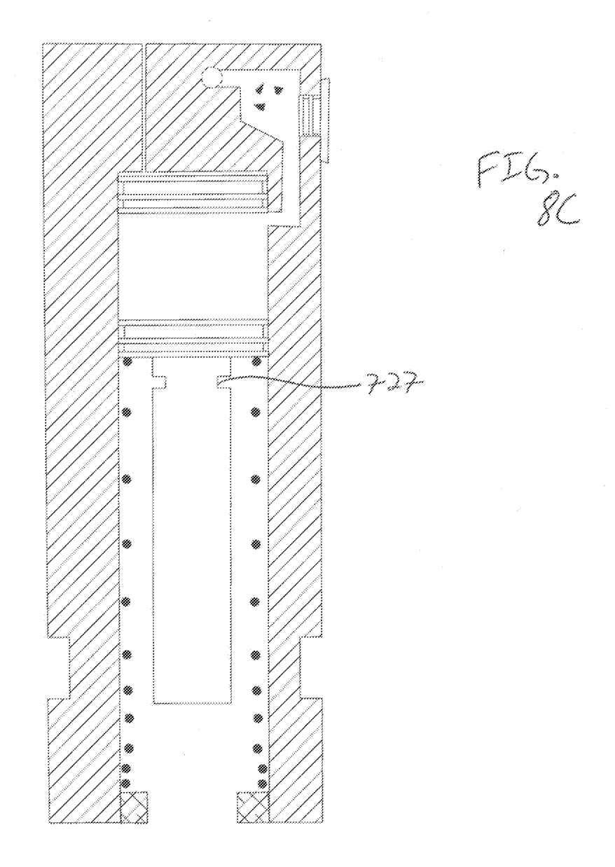

FIGS. 8A-8D illustrate a mixing and delivery device in accordance with a third embodiment 700. Device 700 has a single barrel 702 arranged to hold a liquid component 745 and a separate side chamber 750 that holds a lyo component 747. In this embodiment, the liquid component 745 passes through side chamber 750 during injection thereby dissolving the lyo 747 during the injection stroke. This type of device works well when it is known that the liquid component 745 will dissolve the lyo 747 quickly enough that it can be done during the injection stroke.

As seen in FIG. 8A, device 700 includes a first barrel portion 710 within an outer case 705. First barrel portion 710 has a stopper 715 and a plunger seal 720 within first barrel portion 710, where plunger seal 720 is movable using plunger 725 based on a spring 770 that is compressed within first barrel portion 710. In particular, plunger 725 has plunger notches 727 that are able to hold spring guard cap 775 so as to keep spring 770 from extending. Further, spring guard base 740 similarly keeps spring 770 from extending.

In between stopper 715 and plunger seal 720 is a liquid component 745. Liquid component 745 is able to pass from first barrel portion 710 to side chamber 750 using communication channel 755. In particular, when the components of first barrel portion 710 are in a first position as illustrated in FIG. 8A, communication channel 755 is plugged by stopper 715. However, as the liquid component 745 is pressed by plunger seal 720, the generally incompressible liquid also pushes against stopper 715, displacing it. This is similar to the plunger/stopper movements within barrel 110 of FIG. 1. When the spring guard component is released, the plunger moves forward, which also causes the stopper to move forward, thereby opening the communication channel. Further, communication channel 755 is far enough from the top of first barrel portion 710 so as to allow stopper 715 to pass over communication channel 755 and clear the opening of communication channel 755. Once stop 715 has been pressed by the incomprehensible liquid past communication channel 755, the liquid component 745 may pass into the side chamber 750.

Communication channel 755 connects first barrel portion 710 to side chamber 750. Side chamber 750 has a plug 760 within side opening 765 that is usable for loading side chamber 750 with lyo component 747. For example, side chamber 750 may be loaded with a lyo by placing freeze-dried portions of the lyo into side chamber 750 via side opening 765. As liquid component 745 passes through communication channel 755 and into side chamber 750, lyo component 747 readily dissolves into liquid component 745 as it passed through side chamber 750, forming a mixed drug. Further, the mixed drug passes out of device 700 through outlet 770. Accordingly, as seen in FIG. 8D, liquid component 745 is seen as having transferred through delivery channel 769 to outlet 770.

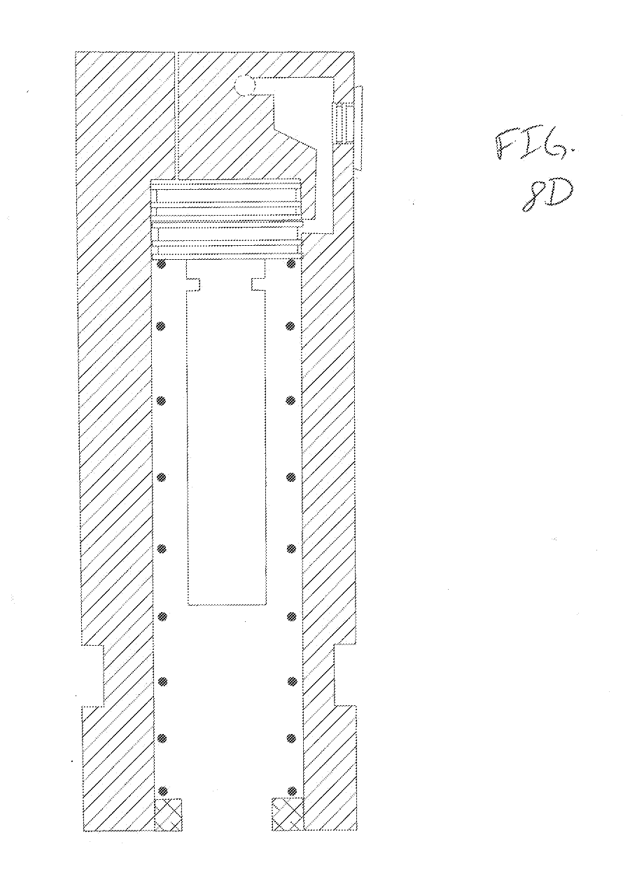

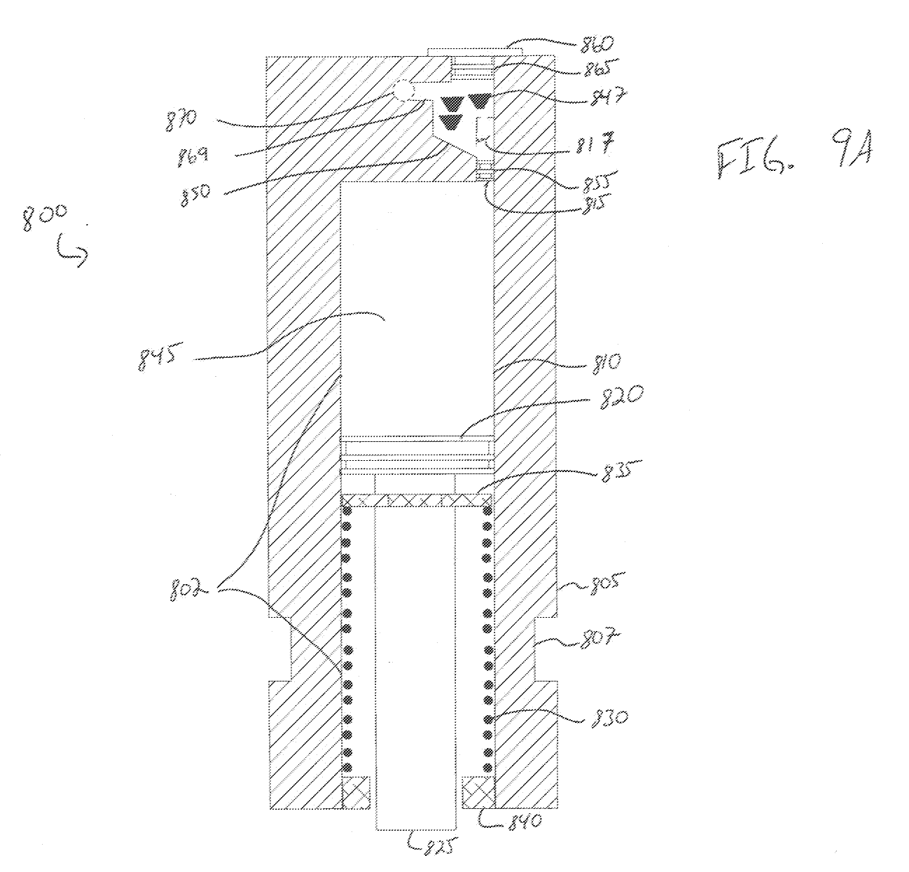

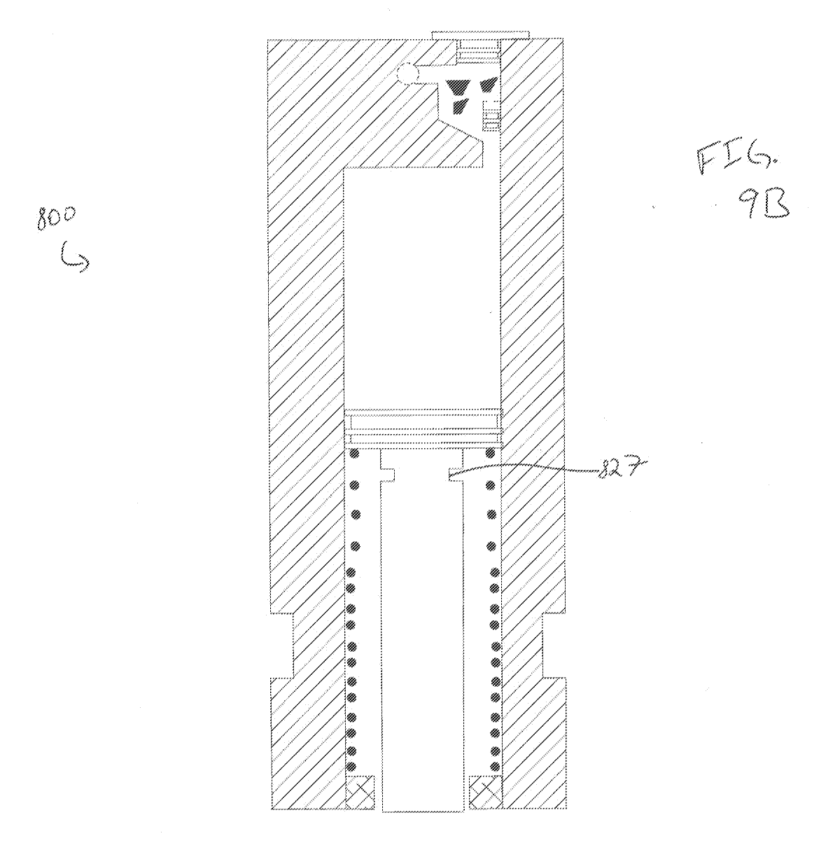

FIGS. 9A-9D illustrate a device in accordance with an embodiment 800 of the present invention. Device 800 is similar to device 700, except the device 700 has a side chamber 750 while device 800 has a sequential chamber 850. Accordingly, in addition to a single barrel 802 that holds a liquid component 885, device 800 has sequential chamber 850 that holds a lyo component 847. Similar to FIGS. 8A-8D, the lyo component 847 is exposed to the liquid component as the liquid component is being ejected from device 800, thereby dissolving the lyo component 847 as the liquid component 845 is passed through the sequential chamber. However, while device 700 provides a liquid component by displacing a stopper, thus revealing a side channel, device 800 has a stopper that itself is sealing an access channel 855, so the mechanism for providing the liquid component 845 to the sequential chamber 850 includes having the stopper 815 displaced into a catch channel 817, thereby opening access channel 855.

As seen in FIG. 9A, device 800 includes a stopper 815 and a catch channel. Similar to the movement of components in barrel 110 of FIG. 1, stopper 815 moves in response to the removal of a spring guard cap. Once the spring guard cap is removed, springs begin to decompress, which propels the plunger distally along the barrel. The movement of the plunger seal presses the liquid component against stopper 815. Since the liquid component is generally incompressible, stopper 815 is pushed into lyo chamber 850. However, rather than interfering with the mixture and/or drainage of the lyo/liquid mixture, stopper 815 is instead pressed against a catch channel 817. Further, the stopper 815 is kept from moving back towards the channel stop by the continuous force placed upon the stopper 815 from a progressing liquid component. As such, the spring activation components of first barrel 810 are similar to those of first barrel 110 of device 100. Similarly, the first barrel 810 has a plunger 825 that is able to engage and displace a generally incompressible liquid component 845. This displacement occurs through the use of plunger seal 820, plunger 825, springs 830, spring guard cap 835, and spring guard base 840 in a manner that matches the similar components in FIGS. 1A-1D. Additionally, lyo component 847 may be loaded into sequential chamber 850 by placing freeze-dried lyo components through a side opening.

Once liquid component 845 is engaged by the spring activation components of device 800, the movement of liquid component 845 displaces stopper 815 as is seen in FIG. 9B. Further, as more of the liquid component 845 passes into sequential chamber 850 and out of device 800, more lyo dissolves, forming a medicament. The amount of lyo provided and the rate of dissolution is commensurate with the amount of liquid and the anticipated rate at which the liquid passes through the sequential chamber so as to maintain a steady concentration of the lyo component in relation to the liquid component in the resulting mixed medicament. The medicament passes out of device 800 through outlet 870. Accordingly, as seen in FIG. 9D, liquid component 845 is seen as having transferred through delivery channel 869 and outlet 870.

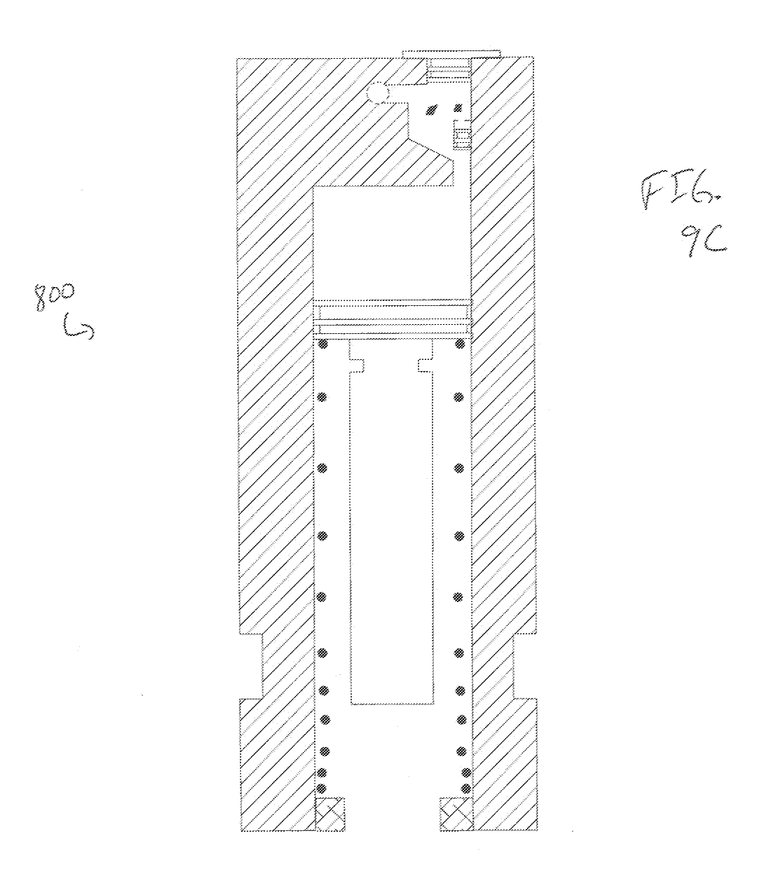

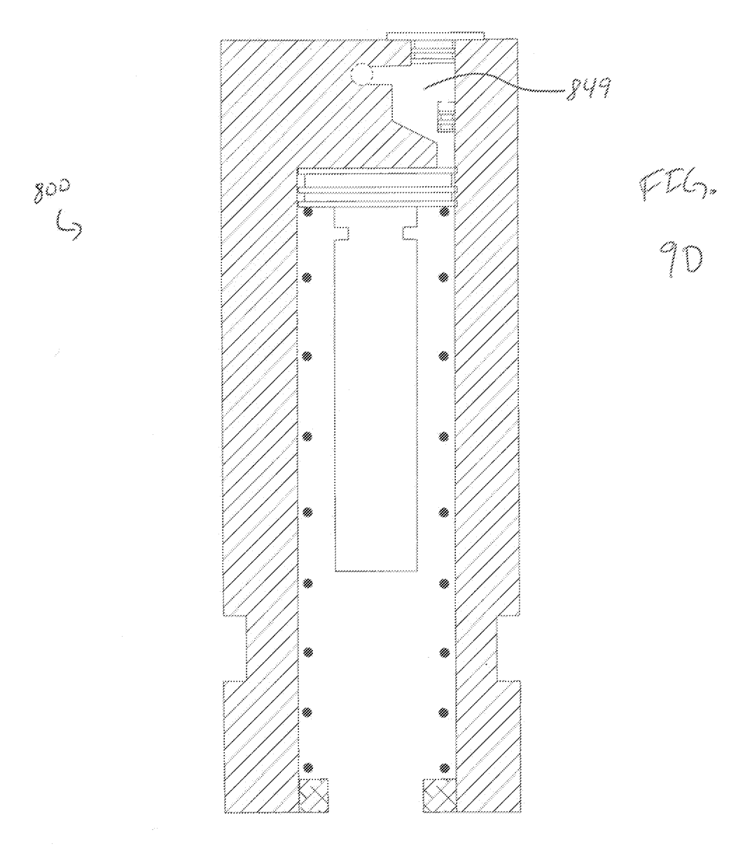

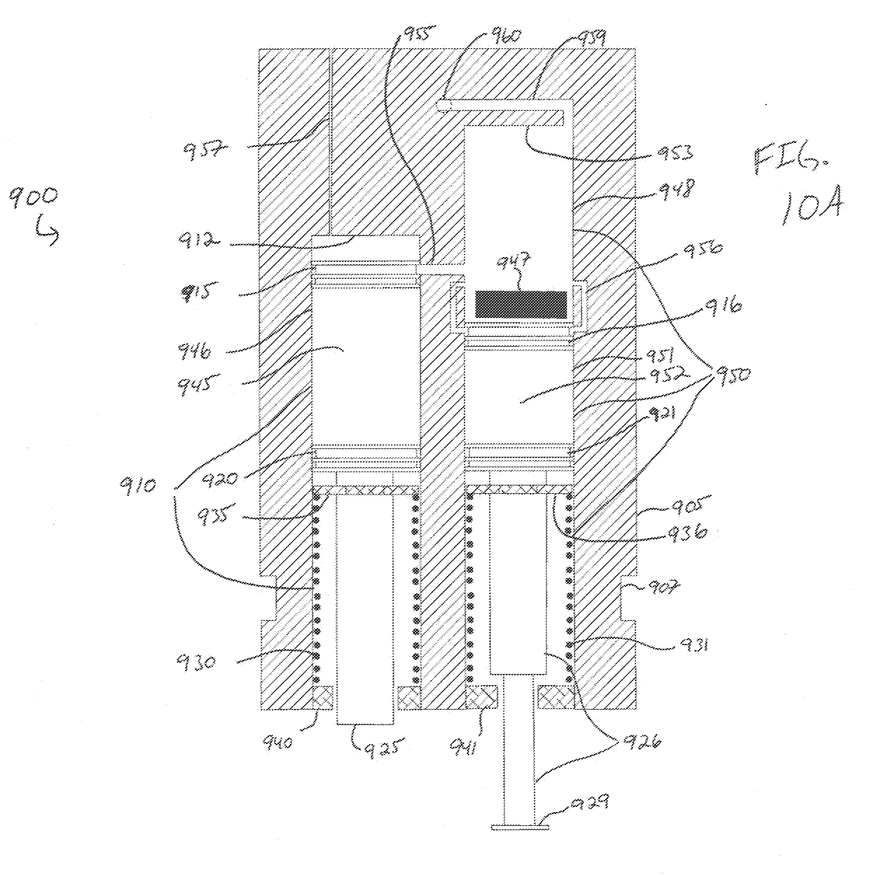

FIGS. 10A-10D illustrate a micro-needle based drug delivery device in accordance with a fifth embodiment 900 of the present invention. In particular, device 900 has two barrels 910, 950 within an outer case 905. While first barrel 910 is substantially the same as first barrel 110 of FIGS. 1A-1D, second barrel 950 differs from second barrel 150 of FIGS. 1A-1D in that second barrel has not one, but two, chambers holding medicament components. In this way, device 900 is particularly well adapted for mixing two liquid components with a third lyo component. As with barrel 110, first barrel 910 contains a liquid component 945 in a liquid storage chamber 946, and that liquid component 945 is provided to second barrel 950 via a communication channel 955 in the same way that liquid component 145 is transferred as described above in FIGS. 1A-1D.

Additionally, second barrel 950 has a second plunger 926 that carries a second plunger seal 921 and a second spring 931 arranged to actuate the second plunger 926. Second plunger 926 may also include notches 928. Similarly to the first spring 930, the second spring 931 may be held in place between a removable spring guard cap 936 and spring guard base 941. The region of the second barrel 950 distal of the plunger seal 921 effectively forms a mixing chamber 948. The mixing chamber 948 is arranged to store a solid component 947 (which in this example may take the form of a lyo). A delivery channel 959 connects the mixing chamber 946 to an outlet 960 which may feed a micro-needle (as described in the incorporated patent application) or other delivery mechanism.

During use, the first plunger may be actuated to deliver the liquid component 945 from the liquid storage chamber 946 to the mixing chamber 948. Alternatively or simultaneously, second plunger 926 may be actuated to deliver the liquid component 952 from the liquid storage chamber 951 to the mixing chamber 948. Within the mixing chamber 948, the liquid components 945, 952 dissolve the lyo component 947 to form a medicament mixture 949 for injection. After the lyo component 947 has dissolved, the medicament mixture 949 may be injected by actuating the second plunger 926.

More particularly, when the plunger 925 is in a first (loaded) position as illustrated in FIG. 10A, the spring guard cap 935 prevents the plunger 925 from moving distally and the stopper 915 is positioned such that it seals communication channel 955 thereby preventing the liquid component 945 from leaking into the mixing chamber 948 in the second barrel 950. When spring guard cap 935 is removed, the compressed spring 930 is released such that the spring pushes plunger 925 and plunger seal 920 distally within the first barrel 910. The liquid 945 is typically substantially incompressible and therefore pushes stopper 915 distally as well to a position where communication channel 955 is opened, allowing fluid communication between liquid storage chamber 946 and the second barrel 950. In the illustrated embodiment, communication channel 955 is proximally far enough from the distal end 912 of first barrel 910 so as to allow stopper 915 to move past communication channel 955 and clear the opening of communication channel 955, thereby allowing liquid component 945 to flow into communication channel 955. This second position is diagrammatically illustrated in FIG. 10B which shows when spring guard caps 935, 936 are removed. In particular, FIG. 10B illustrates the first plunger 925 and second plunger 926 part way through their strokes with the stopper 915 moved to open the communication channel 955. In this second position, liquid component 945 is able to pass from first barrel 910 to second barrel 950 through communication channel 955. The air that previously resided in the space between stopper 915 and the top of first barrel 910 is vented through vent 957. Further, as illustrated in FIG. 10B, liquid component 952 is able to pass through channels 956 to mixture chamber 948.

As the plungers 925, 926 and plunger seal 920 continue movement distally within the first barrel 910 and second barrel 950, the liquids 945, 952 follow the path of least resistance and is thereby forced into the mixing chamber 548 of the second barrel until the plungers 925, 926 come to rest and/or the plunger seal 920 seals off communication channel 955 and/or plunger seal 921 seals off communication with channel 956. In the illustrated embodiment, distal movement of the plungers 925, 926 is stopped when plunger seals 920, 921 come into contact with stopper 915, 916 and distal movement of the stopper 915 is physically constrained by end wall 912 of barrel 910. In the illustrated embodiment, the distance between the end of communication channel 955 and the barrel end wall 912 is selected to substantially match the length of the stopper 915 so that the plunger seal comes to rest adjacent the communication channel 955. This has the advantage of helping ensure that substantially all of the liquid components 945, 952 is ejected from the first barrel 910 which gives very good control over the amount of liquid that is delivered to the mixing chamber 948 of the second barrel. However, it should be appreciated that in other embodiments, the distance between the end of the communication channel 955 and the barrel end wall 912 may vary widely.

When liquid components 945, 952 pass through communication channel 955 and side channel 956 and into the mixing chamber 948, liquid components 945, 952 interacts with solid component 947 (which in this example takes the form of a lyo). Accordingly, lyo component 947 dissolves within liquid components 945, 952. This is diagrammatically illustrated in FIG. 10B which shows the first plunger 925 part way through its stroke. At this point, some (but not all) of the liquid components 945, 952 has been transferred to the mixing chamber 948 and has begun dissolving lyo 947 as diagrammatically illustrated.

FIG. 10C illustrates the drug delivery device 900 in a stage where the first plunger 925 has been fully actuated and the second plunger 926 is constrained from further actuation due to the geometry of plunger base 929. As seen in FIG. 10C, plunger seal 920 engages the stopper 915 and plunger base 929 comes into contact with the spring guard base 940 which prevents further distal movement of the plunger 925. In this stage, the liquid components 945, 952 have been transferred to mixing chamber 948 where they completely dissolve the lyo component 947. Once the lyo 947 has fully dissolved, the resulting medicament mixture 949 is ready for injection. The resulting mixture may then be ejected from the assembly and administered to the patient as a medical treatment by actuating the second plunger 926.

At this stage, both the device 900 and the medicament mixture 949 are ready for injection. The plunger 925 can then be manually actuated by releasing the plunger base 929 from the spring guard base 940 in the same way that plunger base 629 is released from spring guard base 640 as described above in FIGS. 7E and 7F. Once the plunger top 929 is released, both the plunger seal 920 and the stopper 915 move distally under the influence of spring 930 pushing the mixture 949 through delivery channel 959 and outlet 960 which may be coupled to a micro-needle or other suitable delivery device as previously described. When the stopper 915 engages the end wall 953 of barrel 950, the injection is complete.

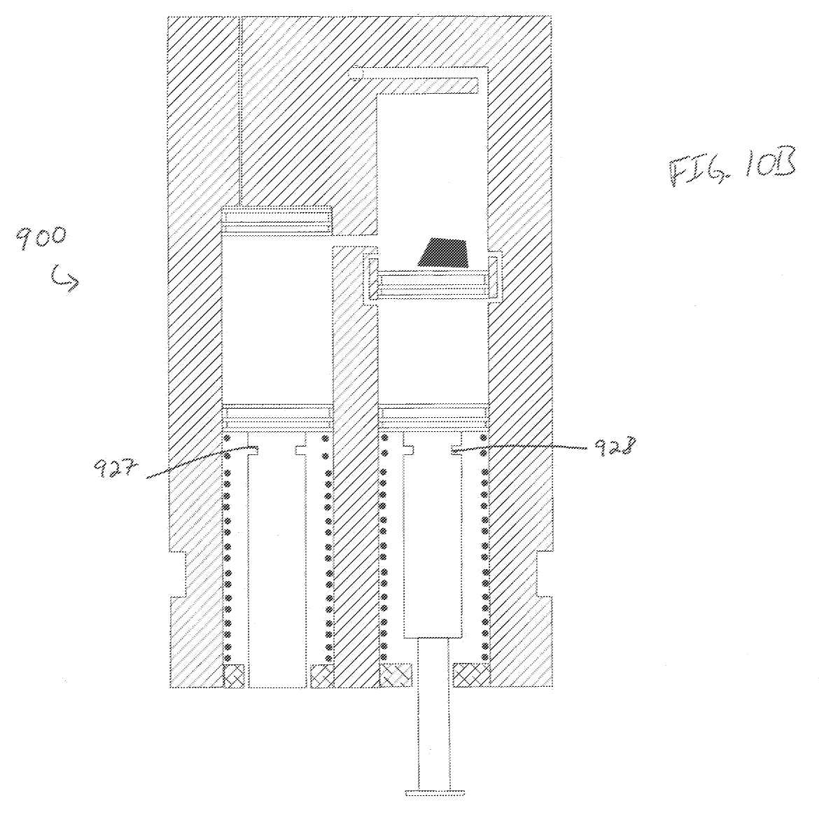

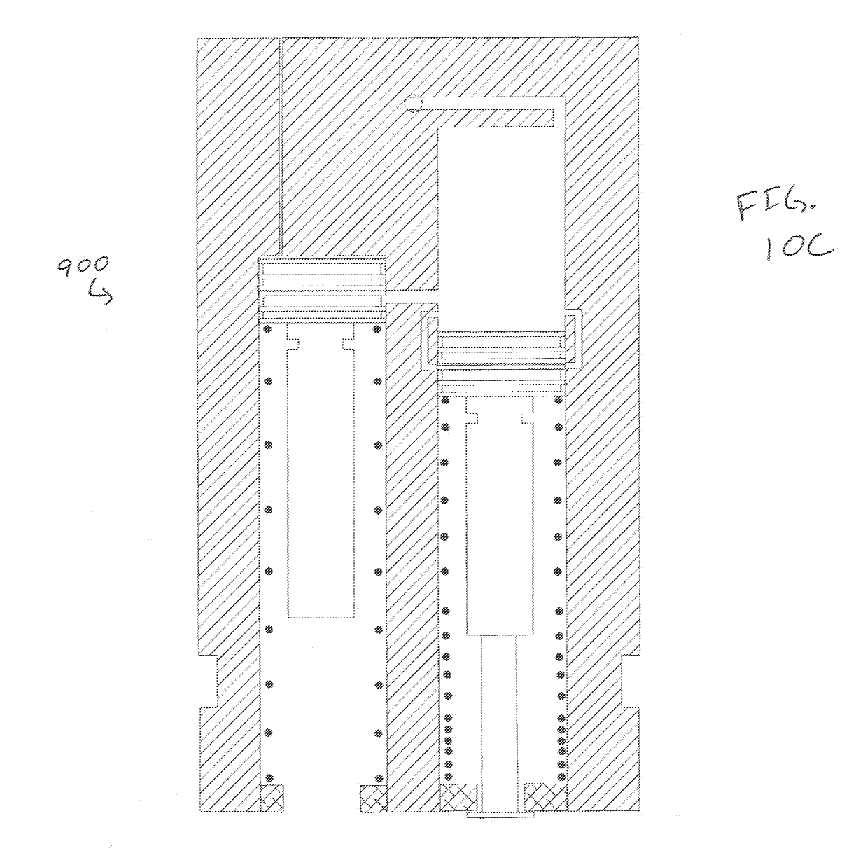

Additionally, embodiments are also provided for retracting a needle into a tube so as to keep it from being able to stick a user or another person after the needle has been used. FIGS. 11-18 illustrate embodiments of a retractable needle apparatus. In particular, embodiments are provided where a plunger seal as discussed above has an additional engagement component that is couplable to a needle case that holds a needle. After the plunger seal is coupled to the needle case, the plunger connected to the plunger seal is able to be retracted, thus moving the needle case and needle along with the plunger and plunger seal. In this way, the needle may be retracted into a barrel by encasing the needle in a needle case then coupling the needle case to a plunger seal.

Further, in embodiments where there is a stopper between the plunger seal and the needle case, the stopper has an engagement case that couples to the engagement portion of the plunger seal. Additionally, the stopper itself has an engagement portion that couples with the needle case, thus sequentially connecting the needle case to the plunger seal via the stopper.

When the plunger seal is retracted, the cover that connects the needle case-needle combination to a delivery device stays in its place while the needle case-needle combination is detached and retracts into a barrel of a delivery device. In additional embodiments, a locking mechanism may be added to a bottom of a plunger connected to the plunger seal. In this way, once the plunger has retracted to a certain point, such as a point where the needle is entirely within the barrel, the plunger may be locked into position to prevent the needle from emerging through the cover and outside of the barrel.

FIGS. 11A-11D illustrate components of a retractable needle apparatus as used in a syringe-type drug delivery device. In particular, FIG. 11A illustrates a connecting adaptor 1105, a needle cage 1110, and a needle 1115. Needle 1115 is used to inject a medicament to a patient. In order to retract needle 1115, a needle cage 1110 is placed around needle 1115 such that the base of needle cage 1110 can interact with an engagement portion that may then be used to retract needle 1115. Further, a connecting adaptor 1105 having a threaded exterior is used to connect the needle cage and needle to a syringe-based device. Connecting adaptor 1105 is placed over needle cage 1110. Accordingly, FIG. 11B illustrates how connecting adaptor 1105, needle cage 1110, and needle 1115 are combined to form retractable needle connector adaptor 1100. Additionally, FIG. 11C illustrates placement of connecting adaptor 1105 atop device 1120. As seen in FIG. 11C, connector adaptor 1105 is threaded onto an outlet of device 1120, where the outlet has internal threading that matches the external threading of connector 1105. Further, plunger seal 1125 is illustrated in FIG. 11D with an engagement portion 1130. In particular, engagement portion 1130 fits within needle cage 1110. Once engagement portion 1130 fits within needle cage 1110, the retraction of plunger seal 1125 works to retract retractable needle connector adaptor 1100.

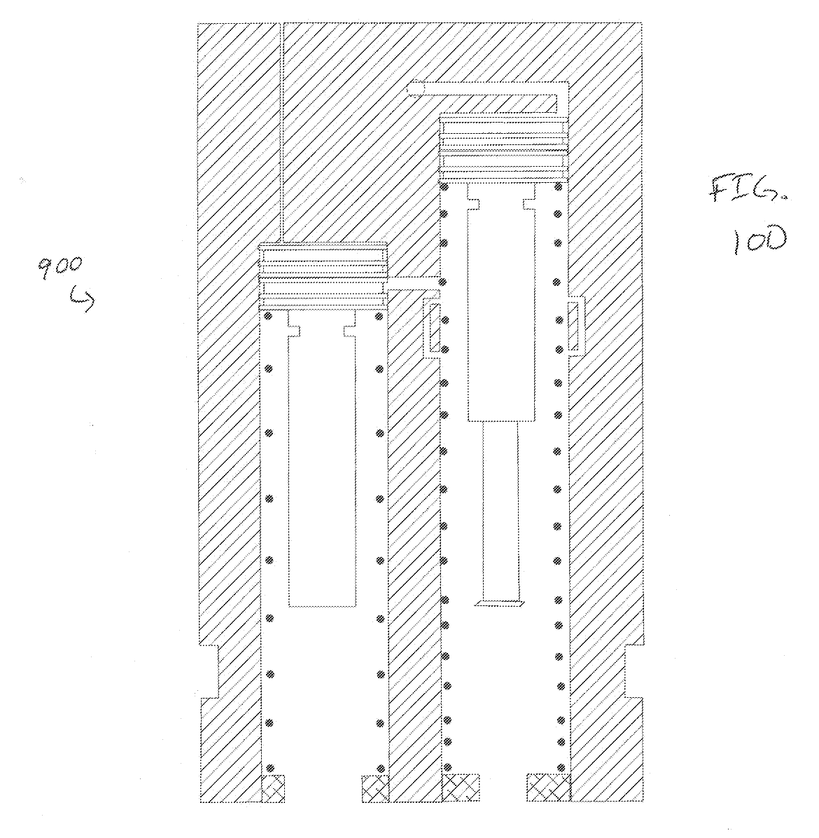

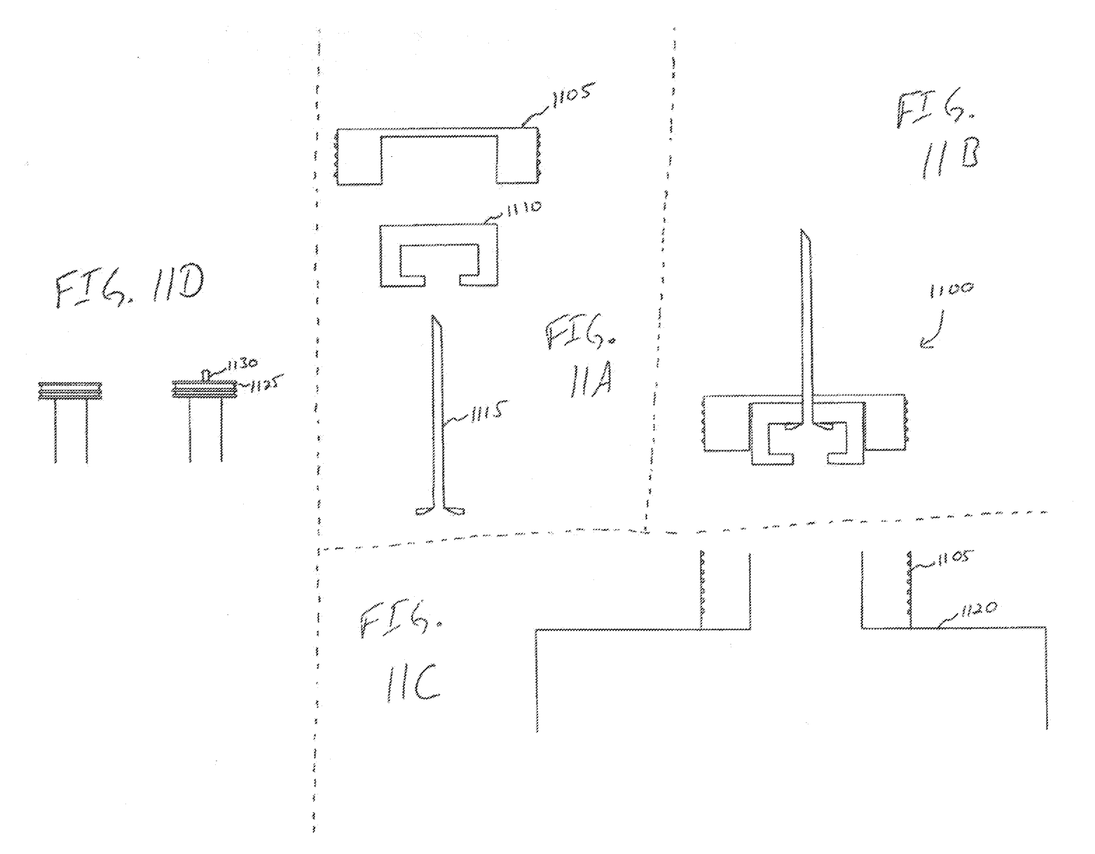

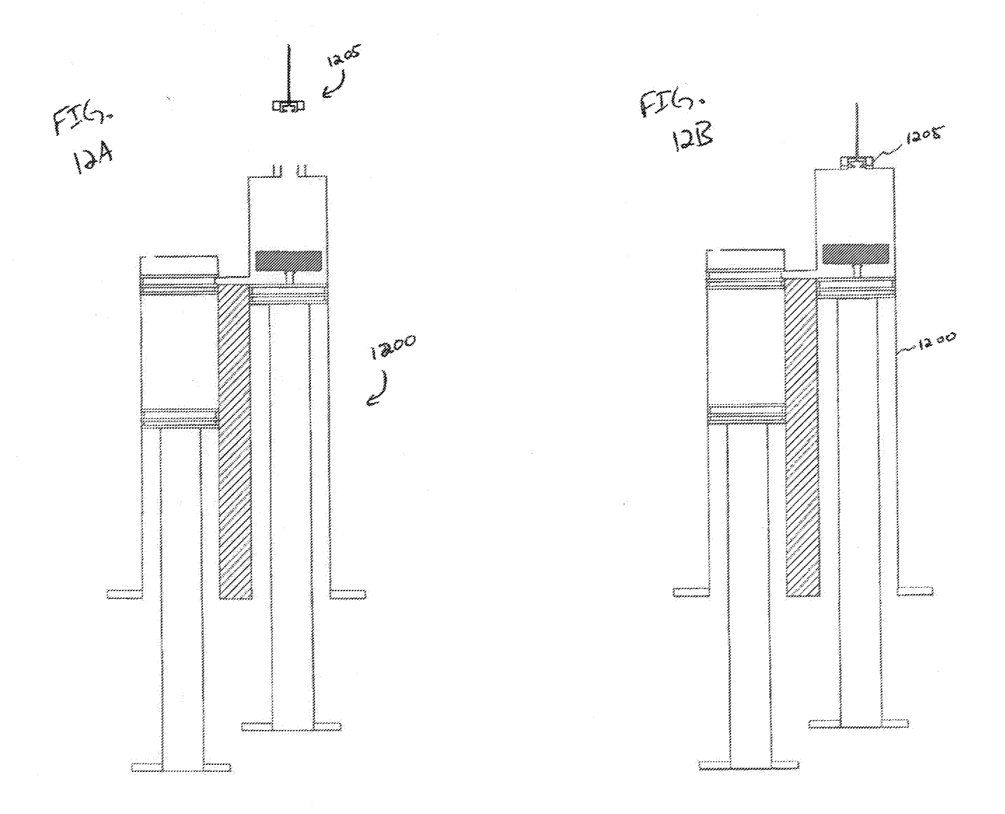

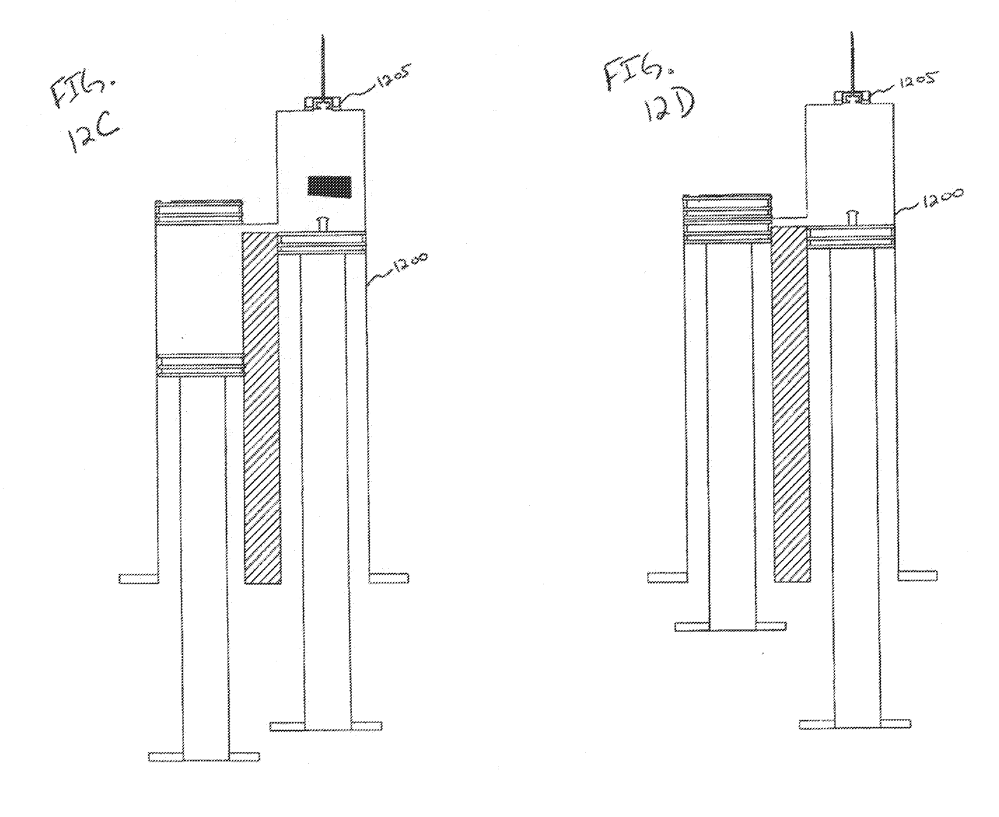

FIGS. 12A-12F illustrate use of a retractable needle apparatus 1205 in accordance with a device 1200 as provided in embodiments of the present invention. Retractable needle apparatus 1205 is the same as 1100 as discussed above. In particular, FIG. 12A illustrates retractable needle apparatus 1205 prior to its securing to device 1200. In particular, device 1200 is a syringe-based drug delivery device having two barrels and is used to mix a liquid component and a lyo component. FIG. 12B illustrates retractable needle apparatus 1205 as combined with device 1200. Retractable needle apparatus 1205 is combined with device 1200 by a user. In one example, a user attaches retractable needle apparatus 1205 by threading apparatus 1205 to the internal outlet threads of device 1200. In other embodiments, the securing mechanism may be an adhesive or a type of mechanical lock, such that a connector, such as connector 1105, is attached to an outlet of a device 1200 while leaving a needle cage and needle free to be retractable. FIG. 12C illustrates as a liquid component of a first barrel is brought into communication connection with a lyo component in a second barrel. Further, FIG. 12D illustrates a mixed drug component in the second barrel as the lyo component has dissolved into the liquid component that was transferred from the first barrel to the second barrel. As seen in FIG. 12D, the plunger seal has an engagement component that engages with the needle cage component of retractable needle apparatus 1205 once the medicament mixture has been expelled from the device.

FIG. 12E illustrates the engagement component of the plunger seal of the second barrel combined with the needle cage component of retractable needle apparatus 1205. Further, after plunger seal of the second barrel has combined with the needle cage component of retractable needle apparatus 1205, the needle case-needle combination 1205′ is able to be retracted while connector 1105 remains connected to device 1200. Accordingly, FIG. 12F illustrates retraction of the needle case-needle combination 1205′ via the retraction of the plunger seal having an engagement portion that is coupled to the needle case.

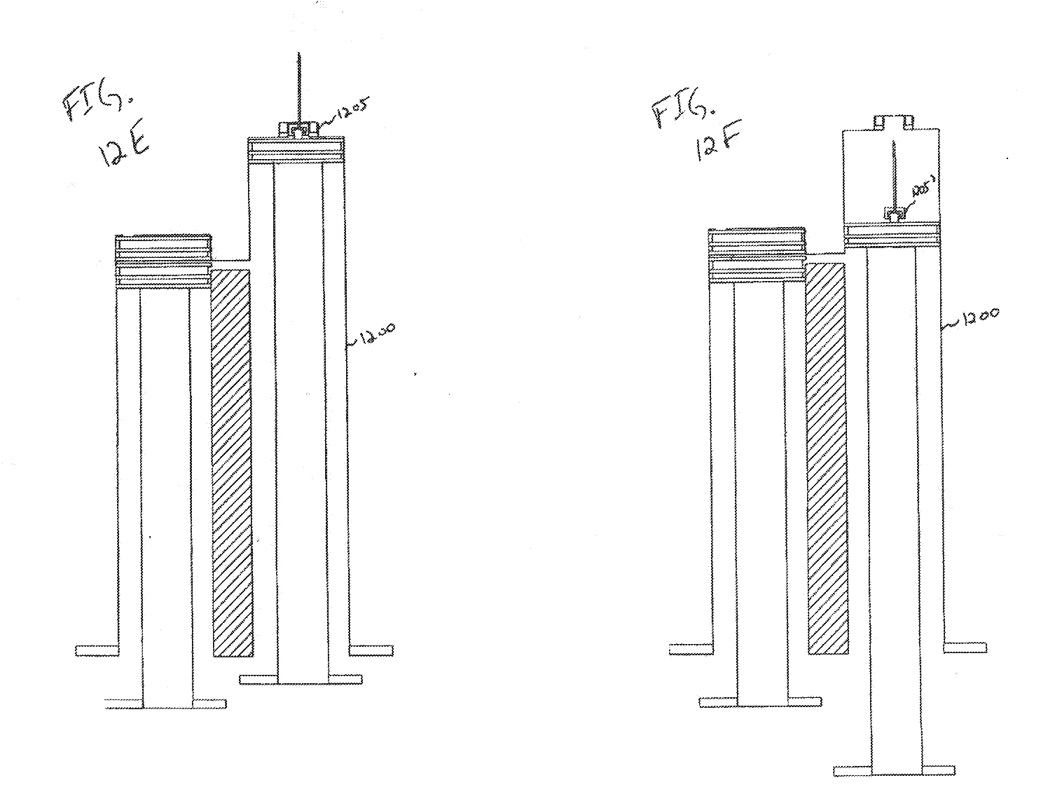

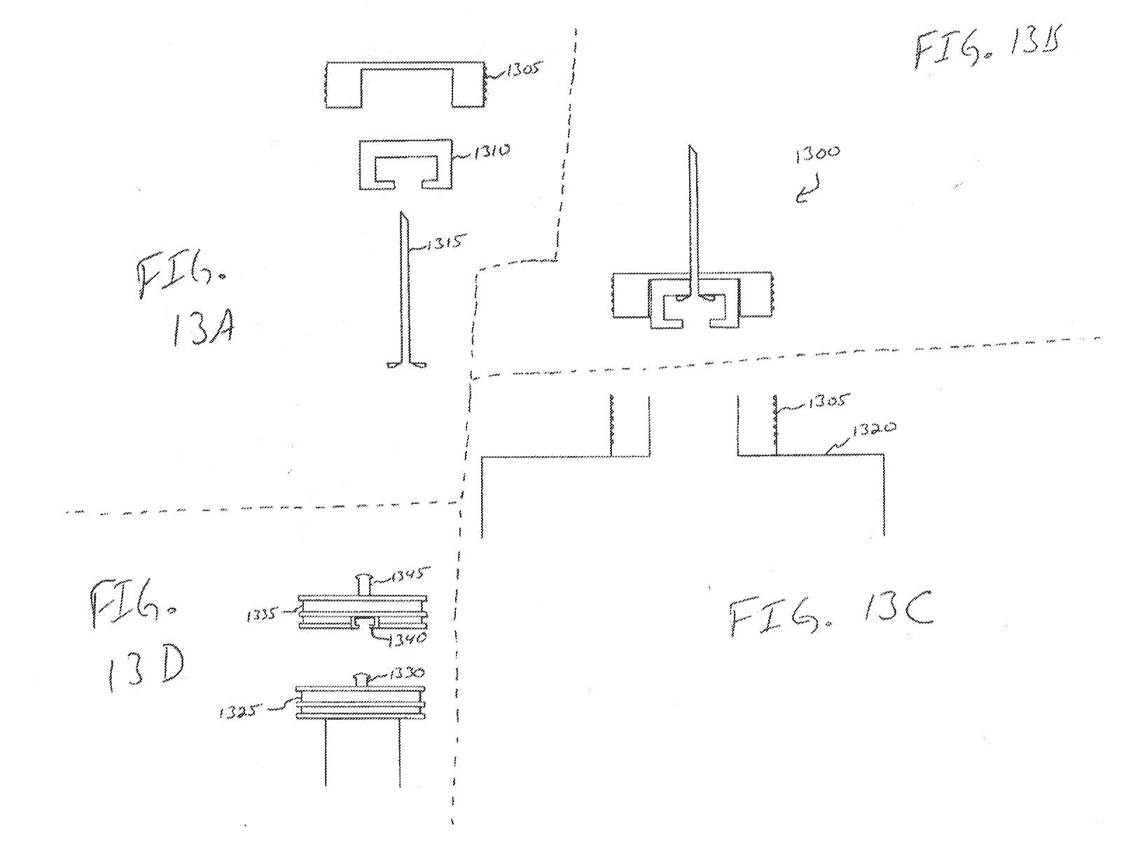

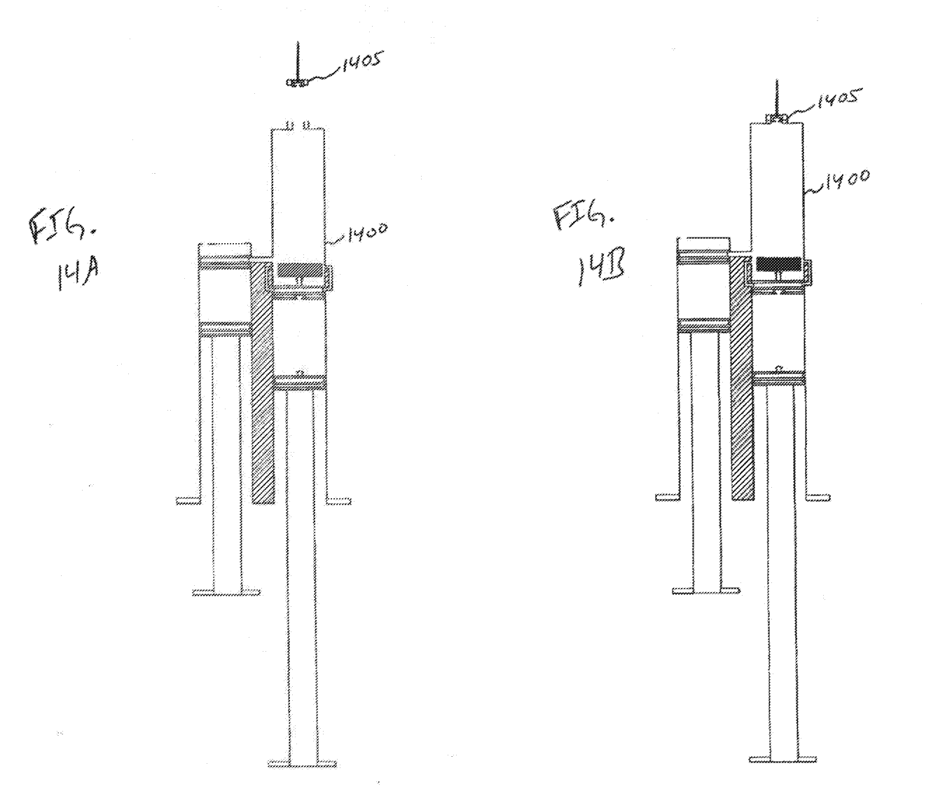

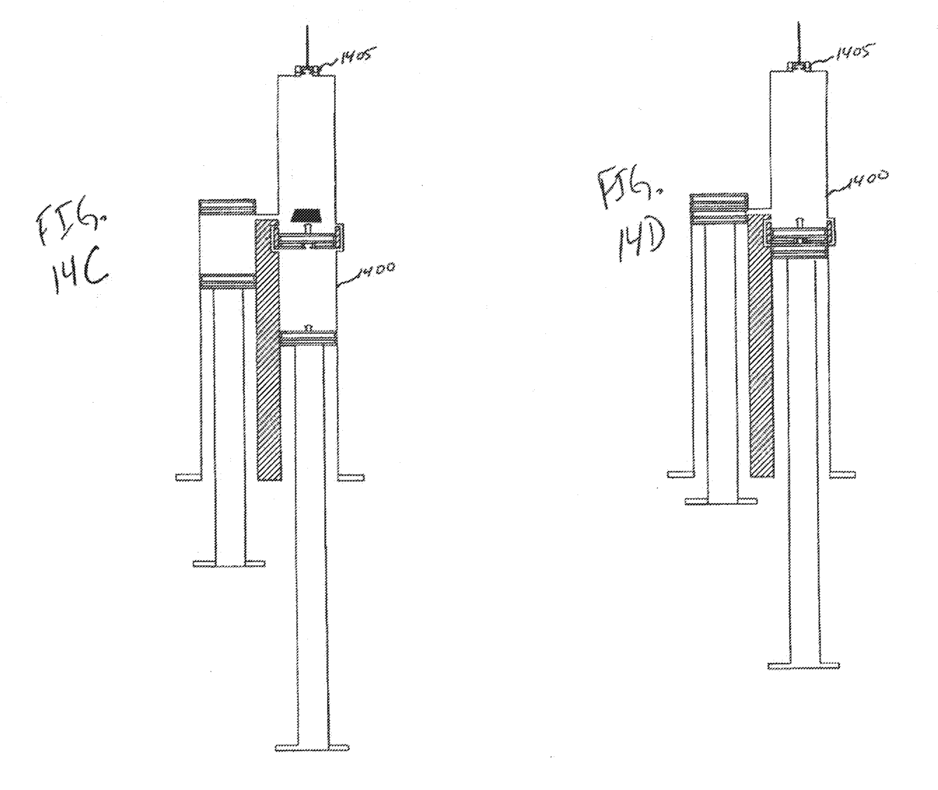

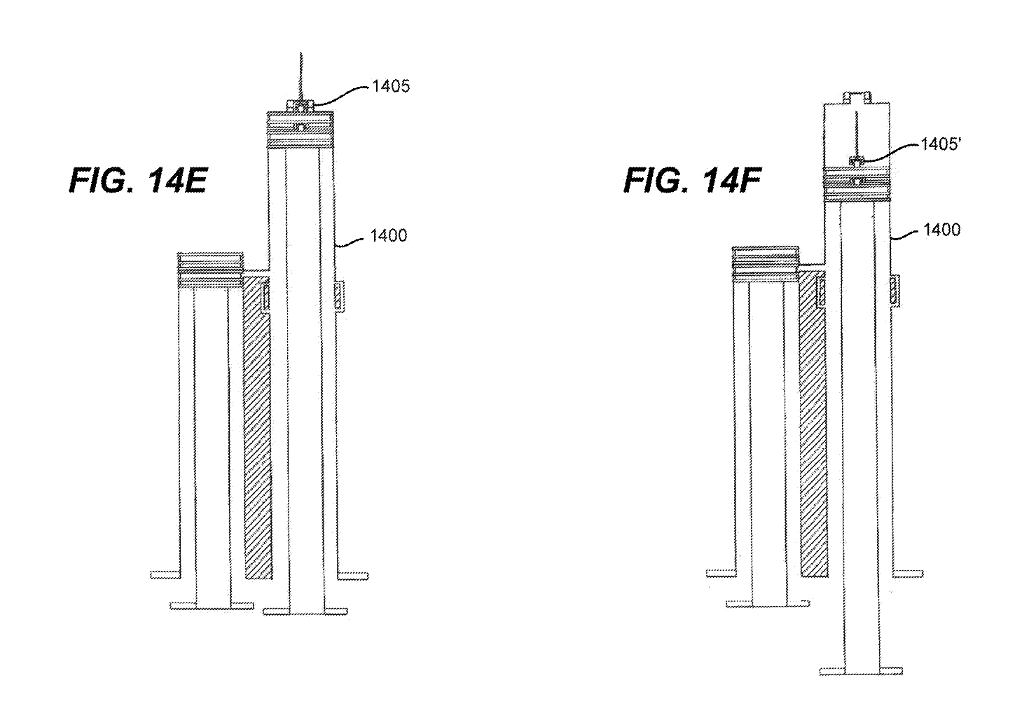

While FIGS. 11A-12F illustrate a retractable needle apparatus that interacts with a plunger seal, FIGS. 13A-14F illustrate a retractable needle apparatus that interacts with a stopper that itself interacts with a plunger seal. Accordingly, FIGS. 13A-13D illustrate components of a retractable needle apparatus 1300 as provided in embodiments of the present invention. FIG. 13A illustrates a connecting adaptor 1305, a needle cage 1310, and a needle 1315. These components are substantially the same as connecting adaptor 1105, needle age 1110, and needle 1115 as discussed in FIGS. 11A-11D. In FIG. 13B, connecting adaptor 1305, needle cage 1310, and needle 1315 are combined to form retractable needle connector adaptor 1300. Additionally, FIG. 13C illustrates placement of connecting adaptor 1305 atop device 1320. As with FIGS. 11A-11D, connecting adaptor 1305 is attached to device 1320 via the external threads of adaptor 1305 and the compatible internal threads of an outlet of device 1320.

Further, plunger seal 1325 is illustrated in FIG. 13D with an engagement portion 1330. Additionally, stopper 1335 is also provided where stopper 1335 includes engagement cage 1340 and engagement portion 1345. In particular, engagement portion 1330 of plunger seal 1325 fits within engagement cage 1340 of stopper 1335. Further, engagement portion 1345 of stopper 1335 fits within needle cage 1310. Once engagement portion 1330 fits within engagement portion 1340 and engagement portion 1345 fits within needle cage 1310, the retraction of plunger seal 1325 works to retract retractable needle connector adaptor 1300.

FIGS. 14A-14F illustrate use of a retractable needle apparatus in accordance with a device 1400 as provided in embodiments of the present invention. In particular, device 1400 is syringe-based and has two barrels and is used to mix two liquid components and a lyo component. The mechanisms used to mix the two liquid components and lyo components are similar to the process shown in FIGS. 10A-10D, except the device in FIGS. 14A-14F is syringe-based while the device in FIGS. 10A-10D is microneedle-based.

Initially, FIG. 14A illustrates retractable needle apparatus 1405 prior to its securing to device 1400 and FIG. 14B illustrates retractable needle apparatus 1405 as combined with device 1400. Retractable needle apparatus 1405 may be combined with device 1400 in ways discussed above. Once retractable needle apparatus 1405 has been combined with device 1400, a liquid component may be transferred from a lower part of the second barrel to the mixing chamber. FIG. 14C illustrates as a liquid component of a first barrel and a liquid component at the base of the second barrel is brought into communication connection with a lyo component in a second barrel. As seen in FIG. 14C, the stopper in the second barrel that previously blocked the liquid component from entering the side channels to the mixing chamber has been moved. In this way, the base liquid component now is able to pass from the bottom of the second barrel to the mixing chamber of the second chamber.

Further, FIG. 14D illustrates a mixed drug component in second barrel as the lyo component has dissolved into the liquid components from the first barrel and a lower portion of the second barrel, respectively, that have been transferred to the upper portion of the second barrel that contained the lyo component. As seen in FIG. 14D, the plunger seal has an engagement component that engages with the engagement cage of the stopper at the base the upper portion of the second barrel. Accordingly, once the plunger seal of the second barrel has pressed all of the liquid component from the bottom of the second barrel to the mixing chamber of the second barrel, the plunger seal is then able to further move to engaged and attach to the stopper of the second barrel. Further, the stopper also has an engagement portion that is able to engage with the needle cage of retractable needle apparatus 1405.

FIG. 14E illustrates the engagement component of the stopper of the second barrel as combined with the needle cage component of retractable needle apparatus 1405. Further, after the stopper has combined with the needle cage component of retractable needle apparatus 1405, and after the plunger seal has been combined with the stopper, the needle case-needle combination 1405′ is able to be retracted. The needle case-needle combination 1405′ is retracted by pulling back on the plunger so that the needle case-needle is drawn down into the second barrel until the needle is within the barrel. Accordingly, FIG. 14F illustrates retraction of the needle case-needle combination 1405′ via the retraction of the plunger seal having an engagement portion that is coupled the stopper which is coupled to the needle case.

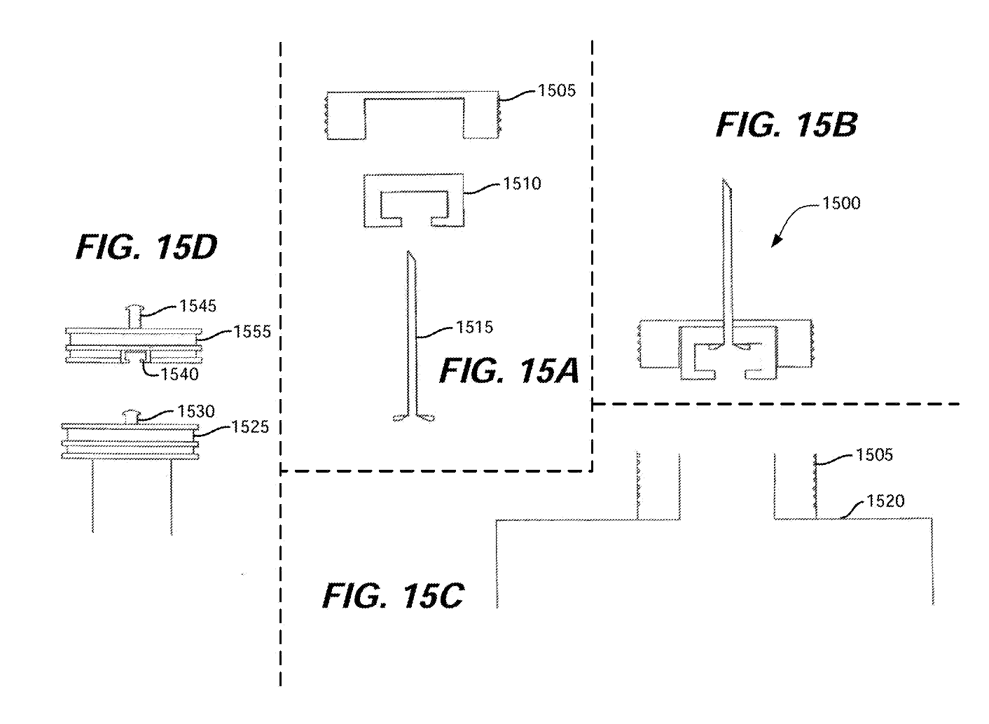

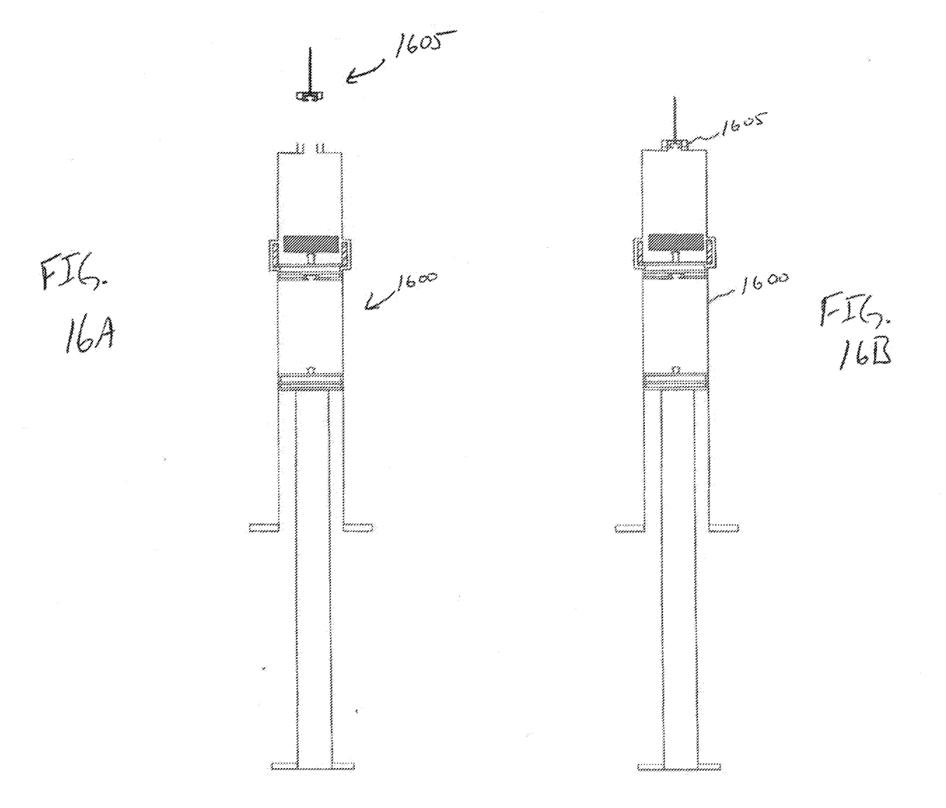

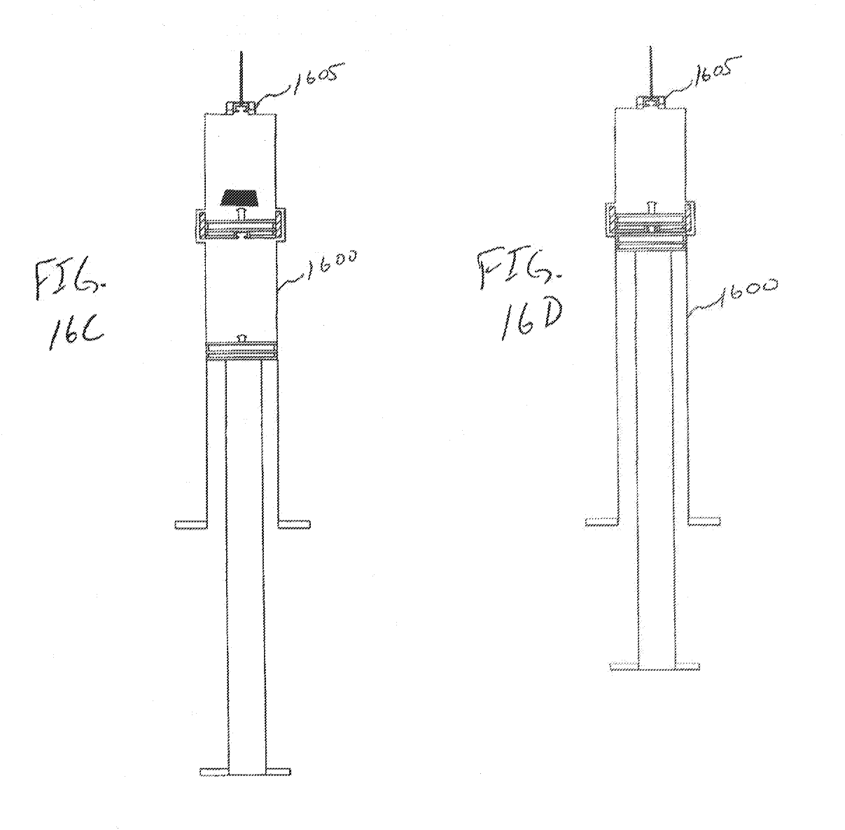

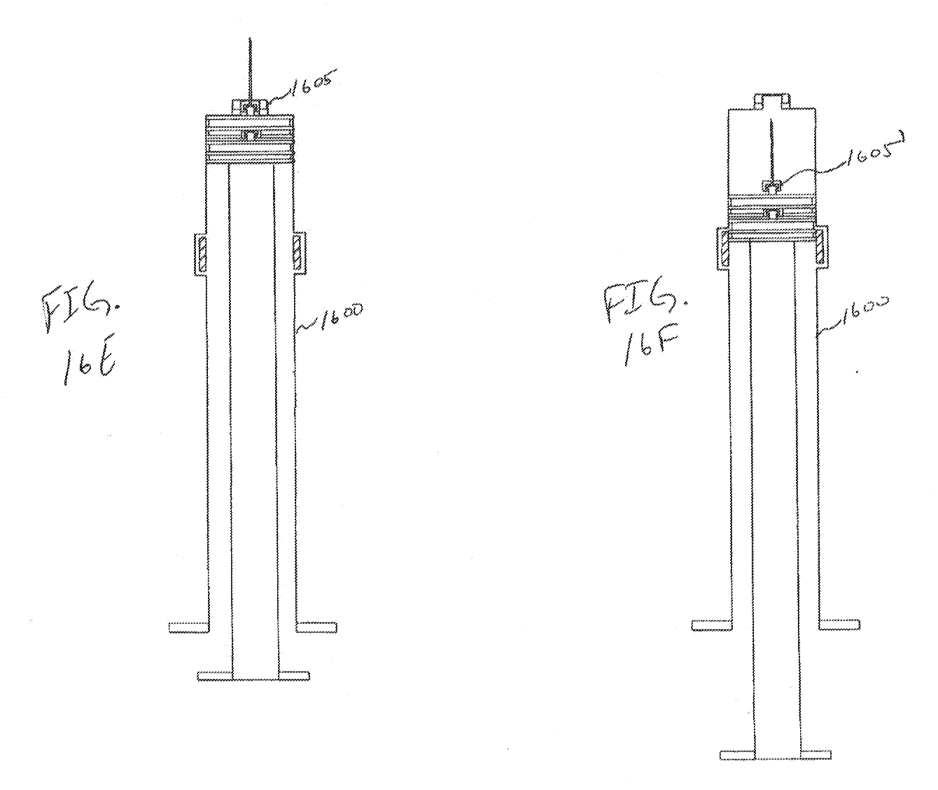

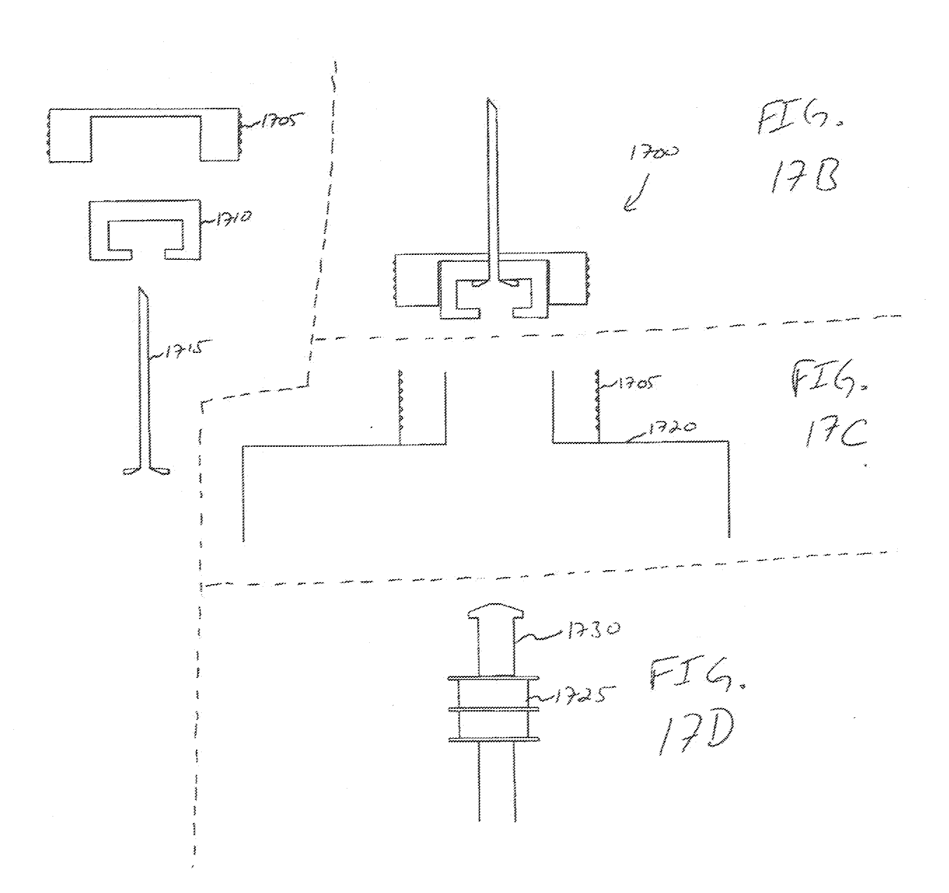

FIGS. 15A-15D illustrate components of a retractable needle apparatus in accordance with a device as provided in embodiments of the present invention. The components of FIGS. 15A-15D are substantially the same as the components of FIGS. 13A-13D as discussed above. Accordingly, FIG. 15A illustrates a connecting adaptor 1505, a needle cage 1510, and a needle 1515. In FIG. 15B, connecting adaptor 1505, needle cage 1510, and needle 1515 are combined to form retractable needle connector adaptor 1500. Additionally, FIG. 15C illustrates placement of connecting adaptor 1505 atop device 1520. Further, plunger seal 1525 is illustrated in FIG. 15D with an engagement portion 1530. Additionally, stopper 1535 is also provided where stopper 1535 includes engagement cage 1540 and engagement portion 1545. In particular, engagement portion 1530 of plunger seal 1525 fits within engagement cage 1540 of stopper 1535. Further, engagement portion 1545 of stopper 1535 fits within needle cage 1510. Once engagement portion 1530 fits within engagement portion 1540 and engagement portion 1545 fits within needle cage 1510, the retraction of plunger seal 1525 works to retract retractable needle connector adaptor 1500.

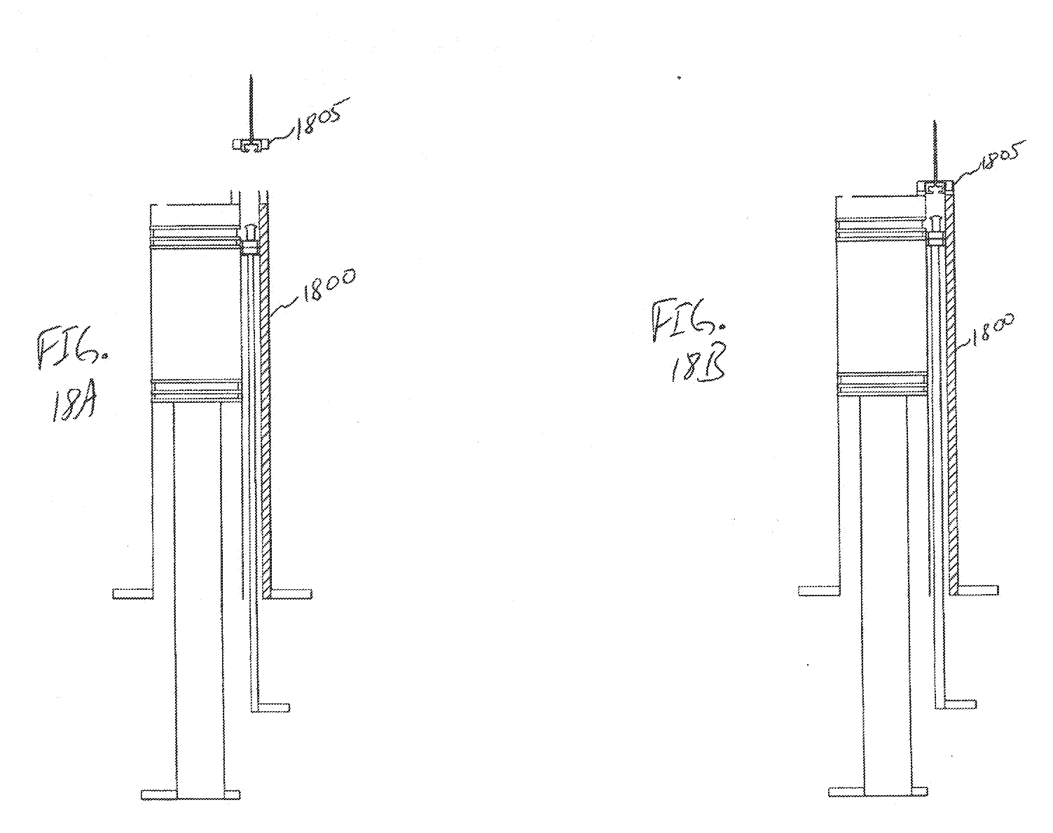

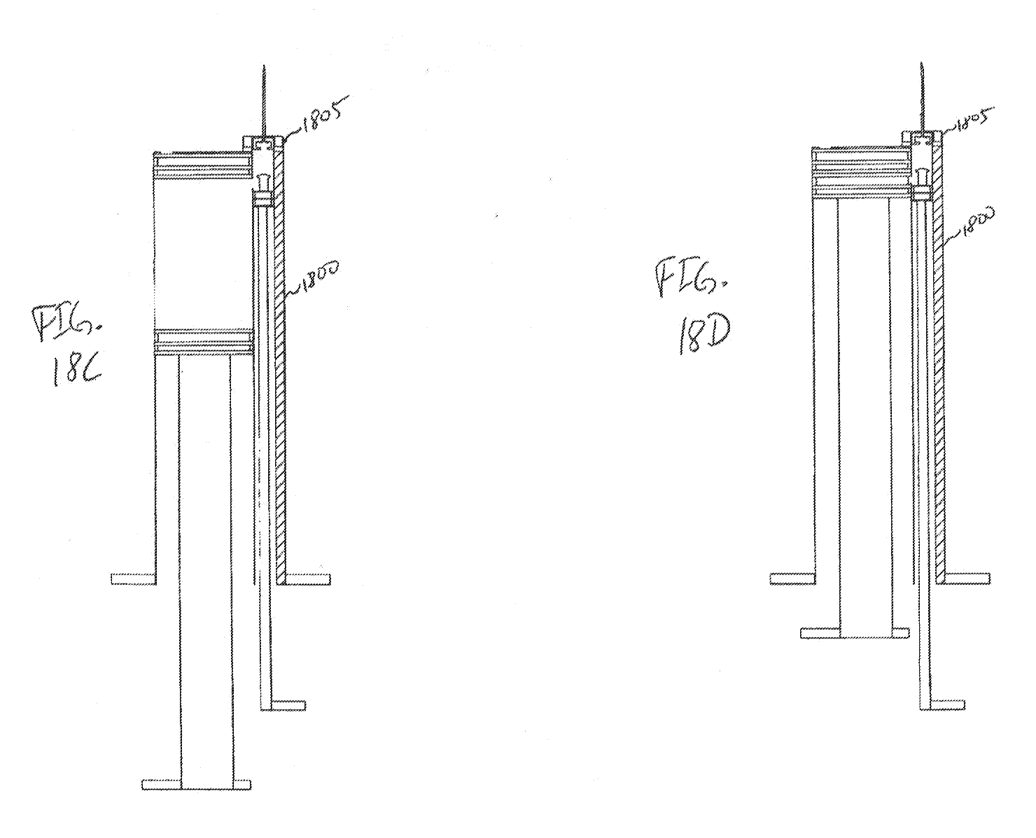

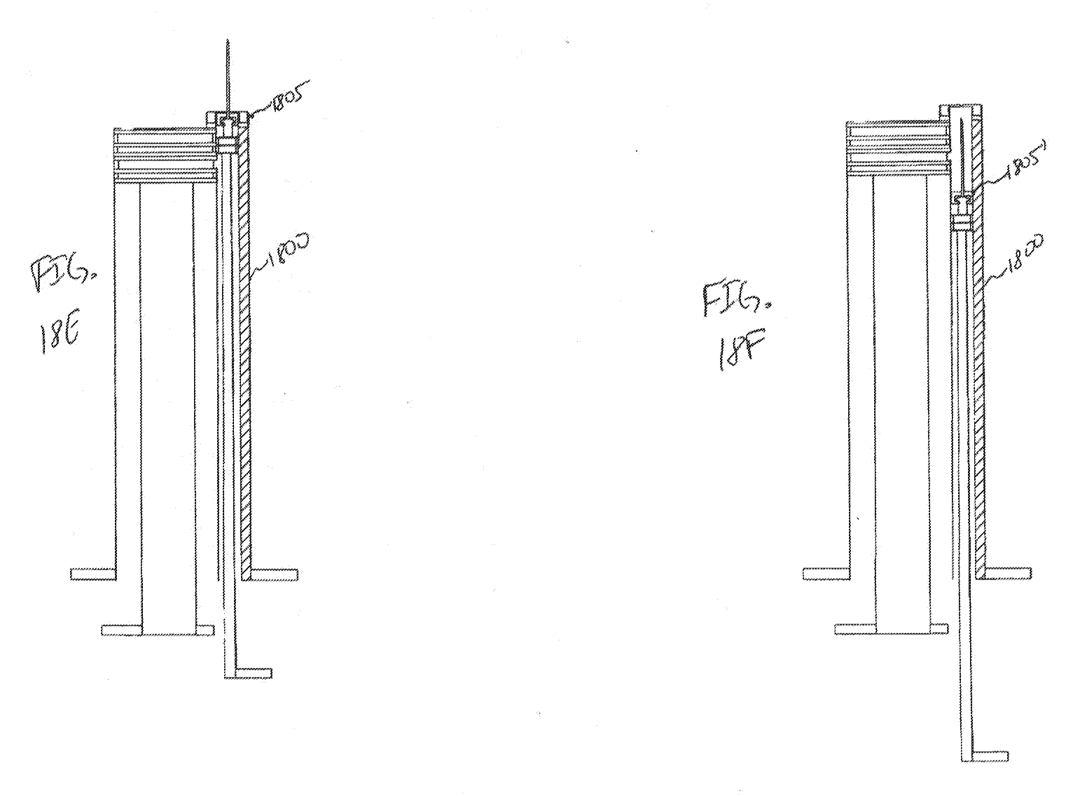

FIGS. 16A-16F illustrate use of a retractable needle apparatus in accordance with a device 1600 as provided in embodiments of the present invention. In particular, the device is syringe-based and has a single barrel and is used to mix two liquid components and a lyo component. While device 1600 is a single barrel device, and device 1400 is a double barrel device, the function of the retractable needle apparatus components is substantially the same within the single barrel of 1600 as it is within the second barrel of device 1400.

FIG. 16A illustrates retractable needle apparatus 1605 prior to its securing to device 1600 and FIG. 16B illustrates retractable needle apparatus 1605 as combined with device 1600. FIG. 16C illustrates as a liquid component of a lower portion of the single barrel is brought into communication connection with a lyo component in an upper portion of the single barrel. Further, FIG. 16D illustrates a mixed drug component in the upper portion of the barrel as the lyo component has dissolved into the liquid components from the lower portion of the barrel that have been transferred to the upper portion of the barrel that contained the lyo component. Once the plunger seal has moved forward enough to dispel the liquid from the lower part of the barrel into the side channels, the plunger seal is able to rest against the stopper. Further, as seen in FIG. 16D, the plunger seal has an engagement component that engages with the engagement cage of the stopper at the base the upper portion of the second barrel. Further, the stopper also has an engagement portion that is able to engage with the needle cage of retractable needle apparatus 1605.

FIG. 16E illustrates the engagement component of the stopper with the needle cage component of retractable needle apparatus 1605. Further, after the stopper has combined with the needle cage component of retractable needle apparatus 1605, and after the plunger seal has been combined with the stopper, the needle case-needle combination 1605′ is able to be retracted. Accordingly, FIG. 16F illustrates retraction of the needle case-needle combination 1605′ via retractable needle apparatus 1605 as combined with the stopper and plunger seal.

While previous retractable needle apparatus examples involve retracting a needle from a primary barrel, alternative forms of a retractable needle apparatuses may be used in syringe-based devices with side channels. As an example, FIGS. 17A-17D illustrate components of a retractable needle apparatus in accordance with a device as provided in embodiments of the present invention. The device has a single barrel and is used to dispense liquid through a side outlet that has a needle retraction component. FIG. 17A illustrates a connecting adaptor 1705, a needle cage 1710, and a needle 1715. These components are substantially the same as adaptor 1105, needle cage 1110, and needle 1115. In FIG. 17B, connecting adaptor 1705, needle cage 1710, and needle 1715 are combined to form retractable needle connector adaptor 1700. Additionally, FIG. 17C illustrates placement of connecting adaptor 1705 atop device 1720. Further, a narrowed plunger seal 1725 is illustrated in FIG. 17D with an engagement portion 1730. Plunger seal 1725 is narrowed so as to accommodate the narrow side channel. In particular, engagement portion 1730 fits within needle cage 1710. Once engagement portion 1730 fits within needle cage 1710, the retraction of plunger seal 1725 works to retract retractable needle connector adaptor 1700.

FIGS. 18A-18F illustrate use of a retractable needle apparatus in accordance with a device 1800 as provided in embodiments of the present invention. FIG. 18A illustrates retractable needle apparatus 1805 prior to its securing to device 1800 and FIG. 18B illustrates retractable needle apparatus 1805 as combined with device 1800. Retractable needle apparatus 1805 may be combined with device 1800 in the same manner discussed above with regard to FIGS. 11A-12F. FIG. 18C illustrates as a liquid component of a first barrel is brought into communication connection with an outlet portion. Further, FIG. 18D illustrates that the liquid portion has been ejected from the device 1800.

Once the liquid has been dispelled from the device, a separate plunger and plunger seal are moved along a side channel to engage the retractable needle apparatus. Accordingly, FIG. 18E illustrates the engagement component of the plunger seal as combined with the needle cage component of retractable needle apparatus 1805. Further, after plunger seal of the second barrel has combined with the needle cage component of retractable needle apparatus 1805, needle case-needle combination 1805′ is able to be retracted. Accordingly, FIG. 18F illustrates retraction of the needle case-needle combination 1805′ via moving the side plunger seal back by pulling back on the side plunger.

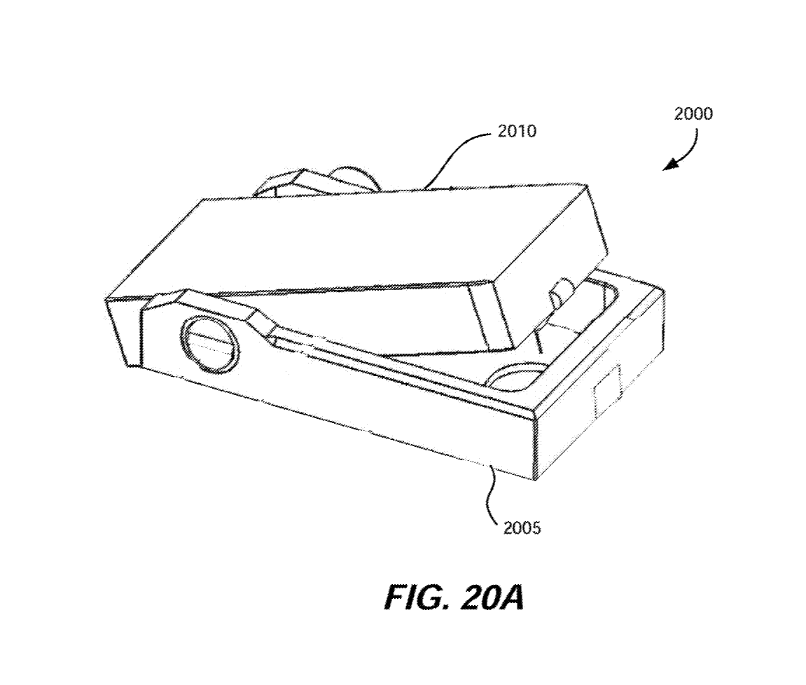

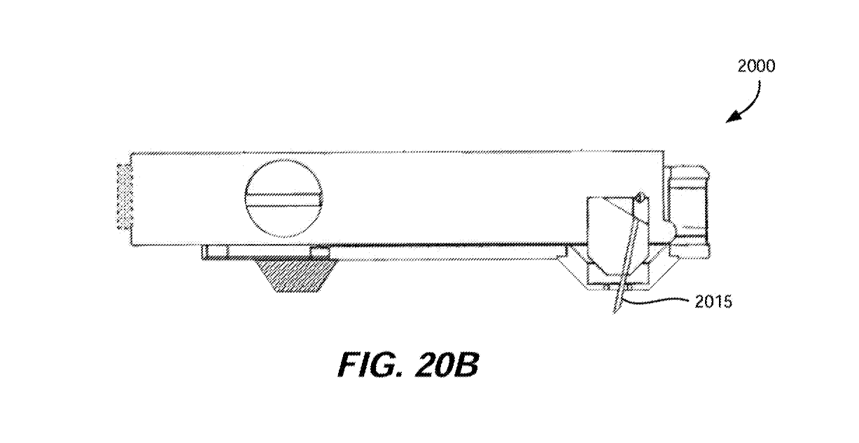

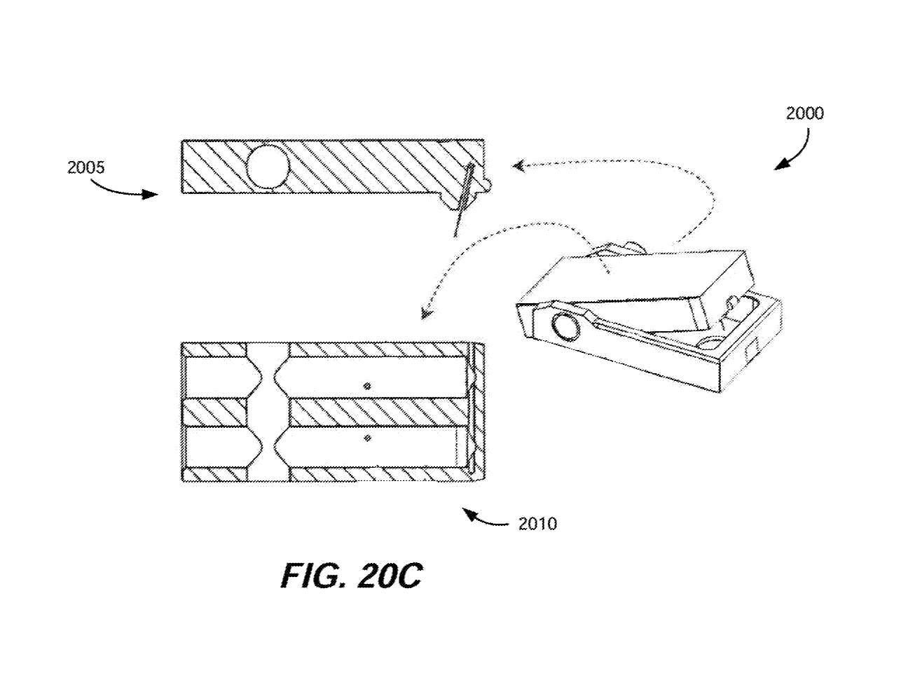

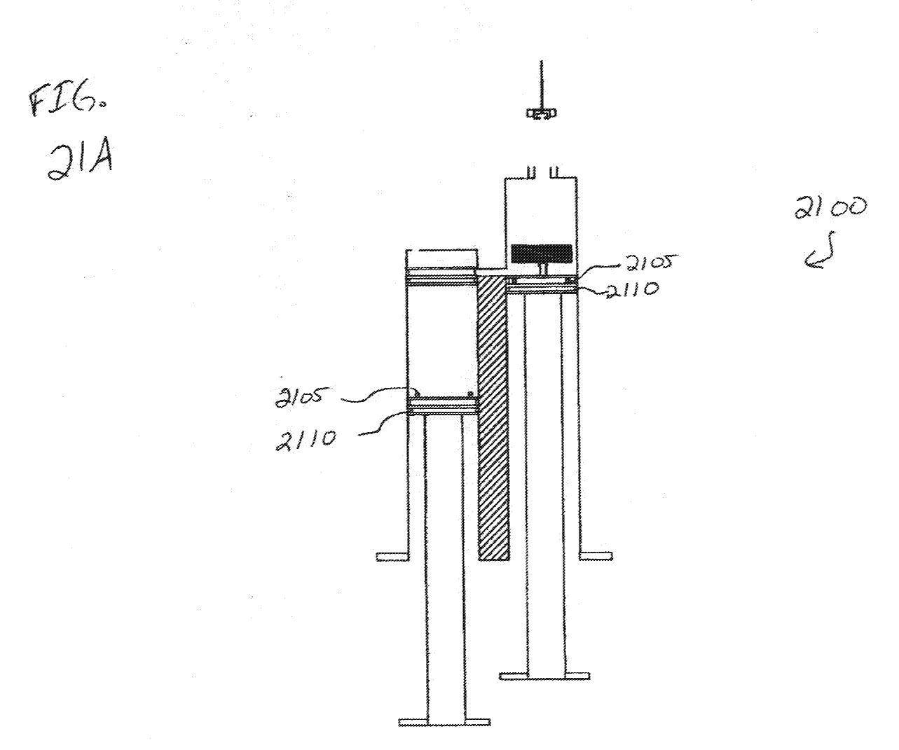

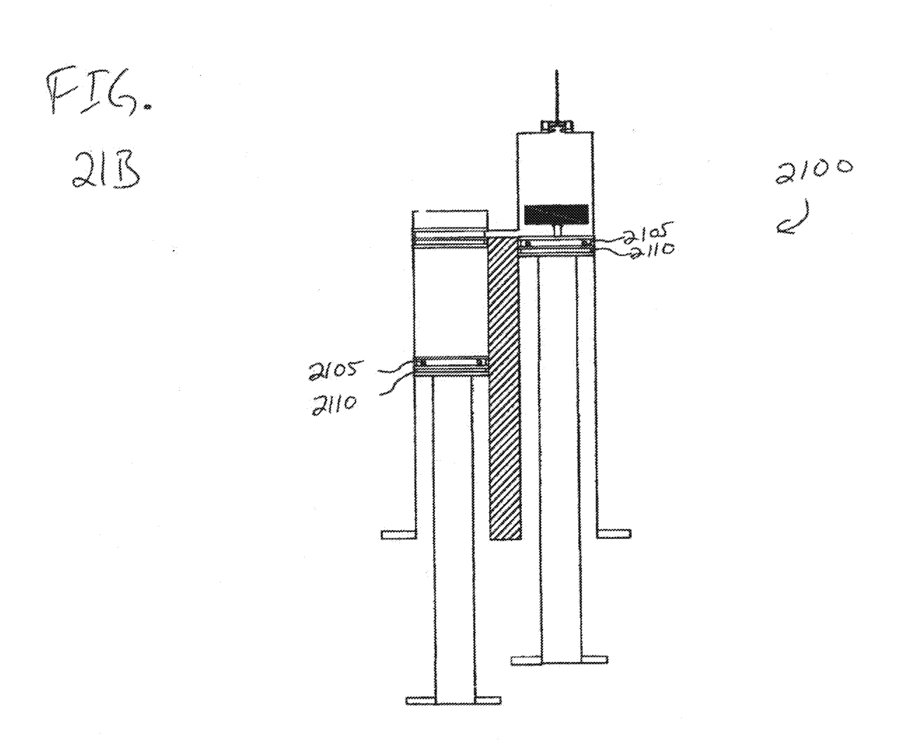

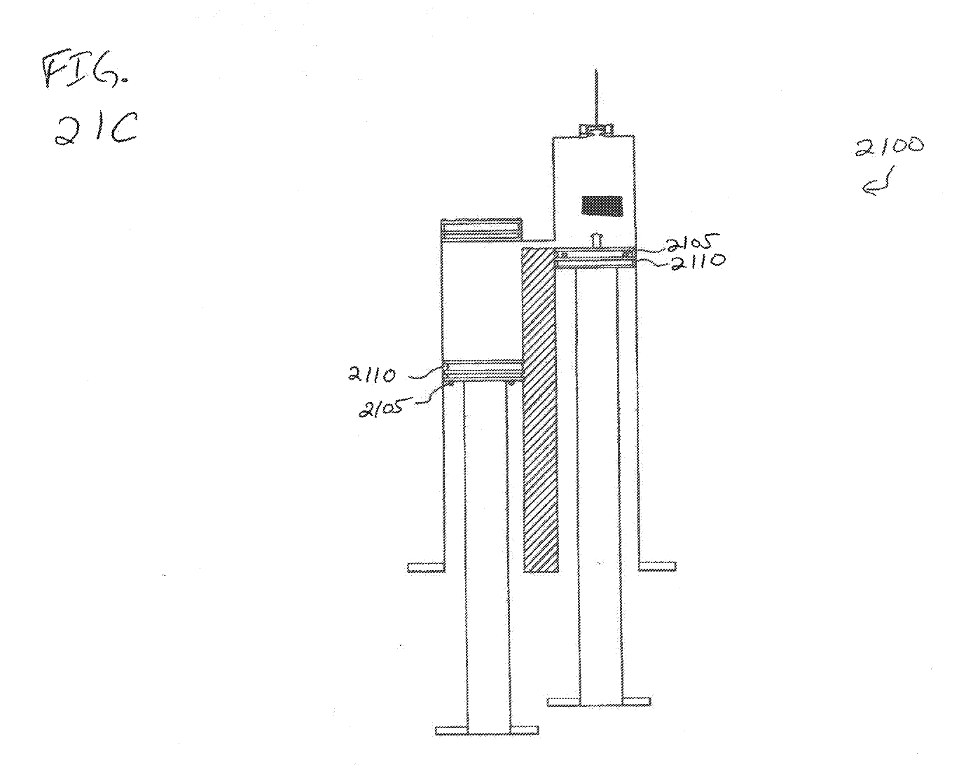

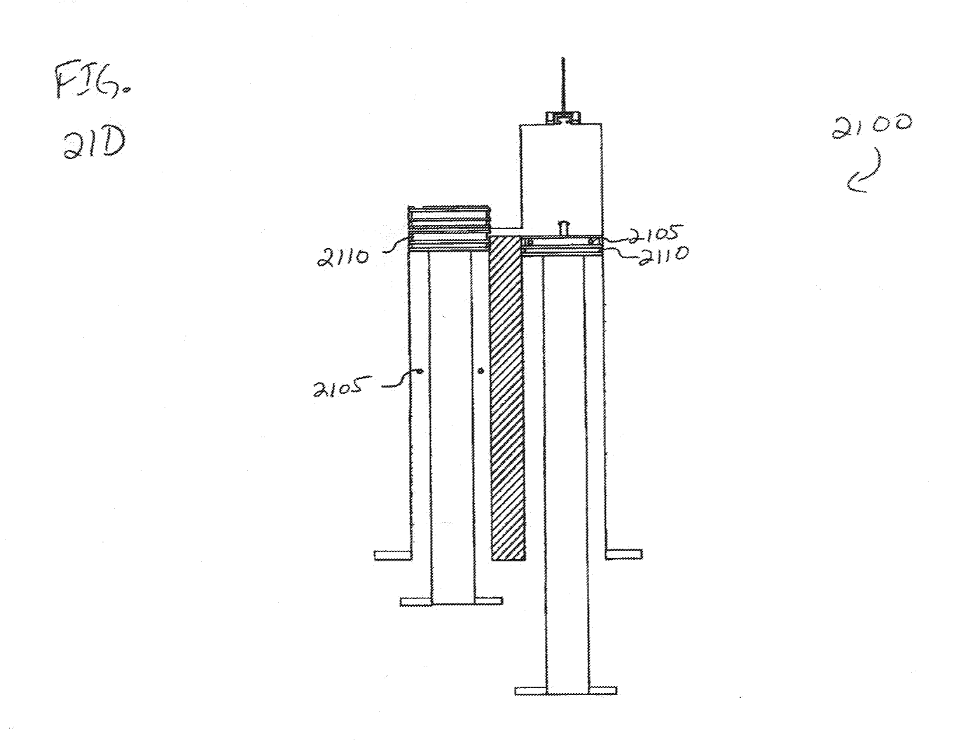

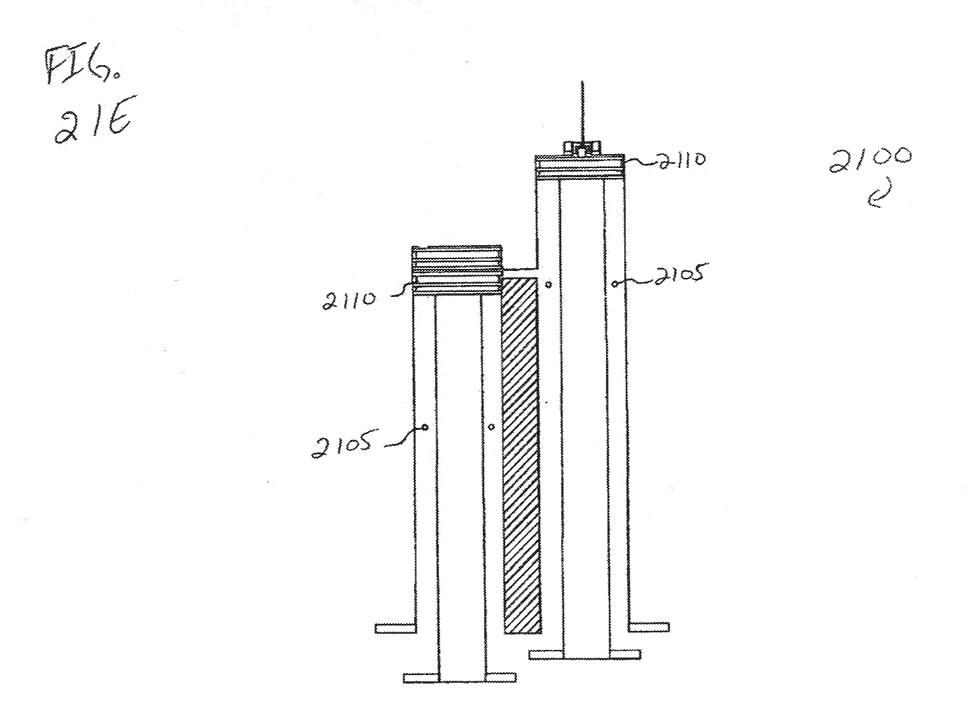

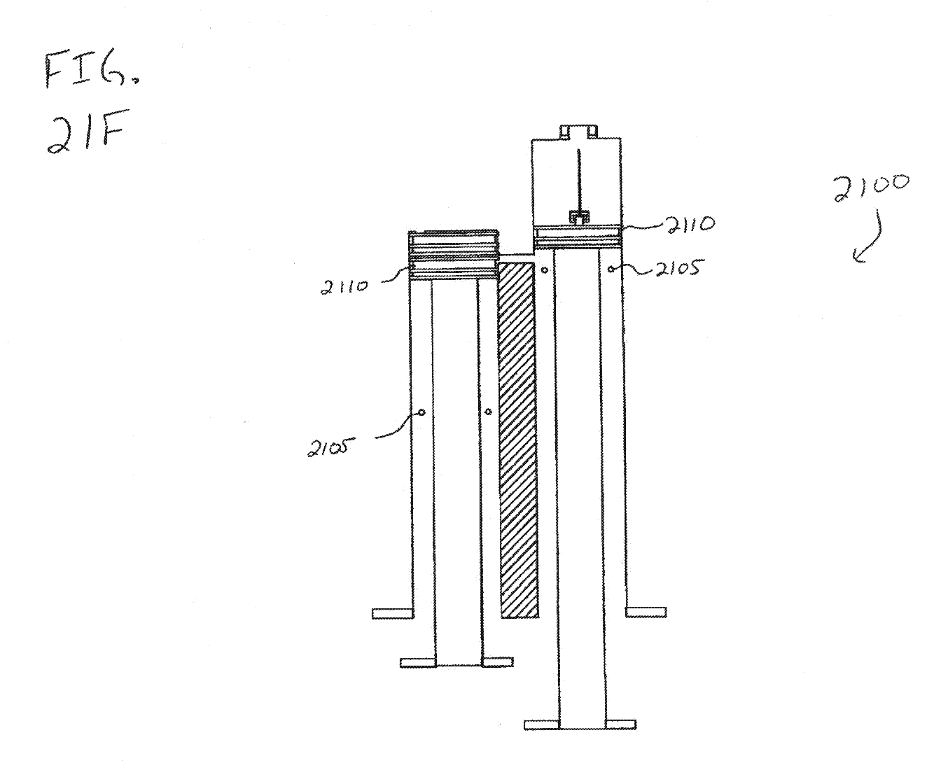

FIG. 19 illustrates a 3-dimensional view of a base 1900 of a microneedle-based transdermal drug delivery device. The base may be connected to a top device such as those described above in embodiments 100, 300. An illustration of a fully formed microneedle-based transdermal drug delivery device 2000 is shown in FIG. 20A. FIG. 20A provides device 2000 as formed by connecting base 2005 to a top portion 2010 using a hinge. Further, FIG. 20B illustrates a side view of fully formed microneedle-based transdermal drug delivery device 2000, including a view of microneedle 2015. Additionally, FIG. 20C illustrates an exploded view of components of microneedle-based transdermal drug delivery device 2000. As seen in FIG. 20C, top portion 2010 is aligned with connecting base 2005 to for device 2000.

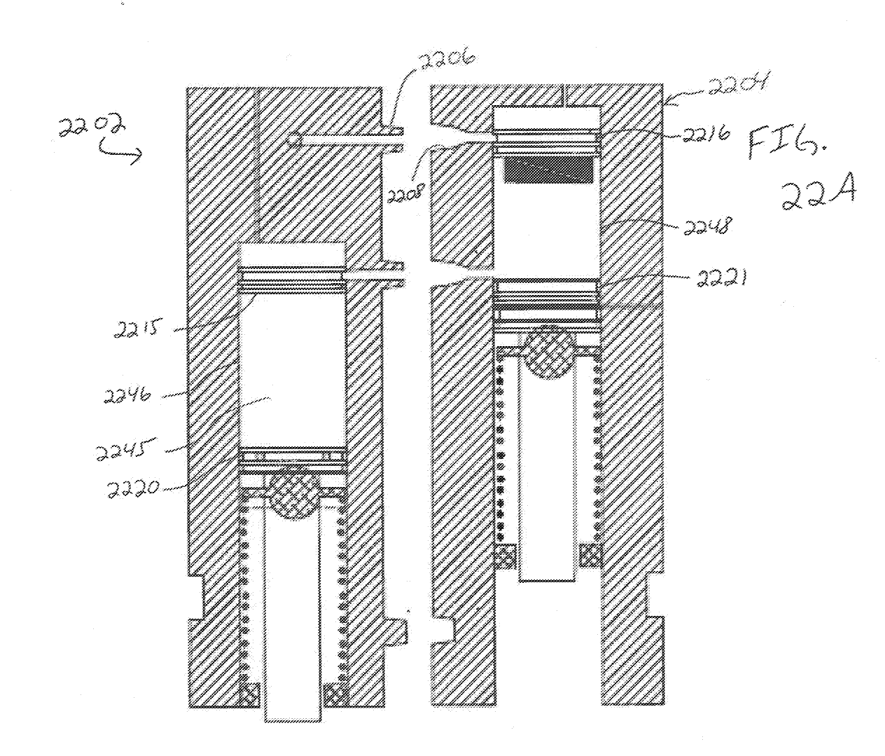

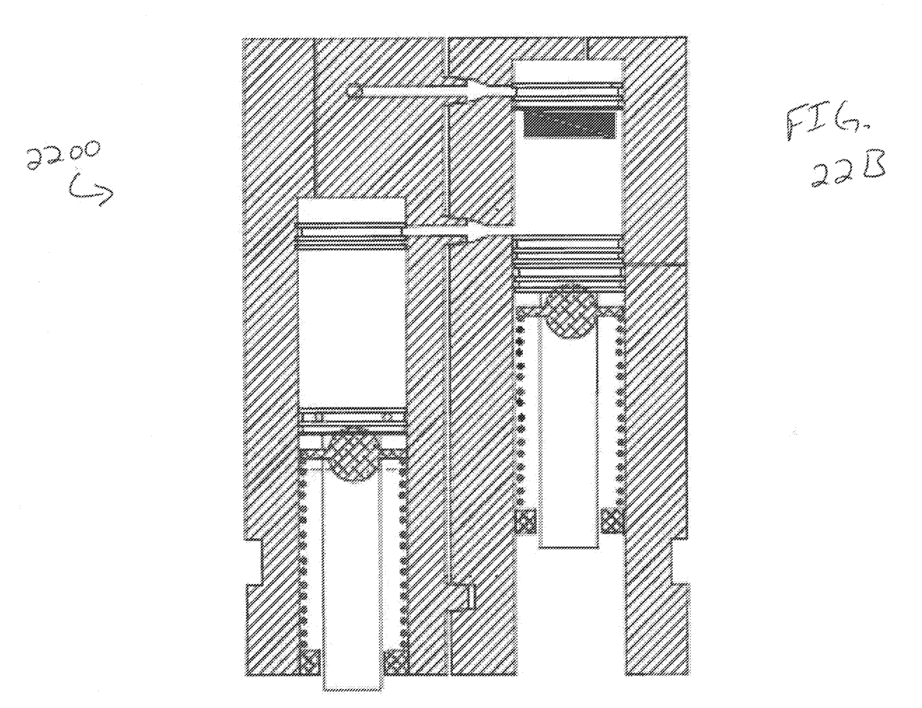

FIGS. 21A-21F illustrate use of a device with a retractable needle apparatus in accordance with embodiments of the present invention. The device 2100 is substantially the same as device 1200 except that device 2100 has fill holes 2105 as well as a retractable needle apparatus.

Accordingly FIG. 21A illustrates retractable needle apparatus prior to its securing to a device 2100. Also seen in FIG. 21A, fill holes 2105 are seen on device 2100. Fill holes 2105 are used to minimize the introduction of air into the liquid chamber. When plunger seal 2110 is pulled below fill holes 2105, the liquid compartment is able to be filled with liquid via fill holes 2105. Further, after the liquid compartment is filled with liquid, plunger seal 2110 may be moved to seal fill holes 2105. This is seen in FIG. 21B. Additionally, FIG. 21B illustrates a retractable needle apparatus as combined with device 2100.

Further, FIG. 21C illustrates as a liquid component of a first barrel is brought into communication connection with a lyo component in a second barrel. Further, FIG. 21D illustrates a mixed drug component in second barrel as the lyo component has dissolved into the liquid components from the first barrel and a lower portion of the second barrel, respectively, that have been transferred to the upper portion of the second barrel that contained the lyo component. As seen in FIG. 21D, the plunger seal has an engagement component that engages with the engagement cage of the stopper at the base the upper portion of the second barrel. Further, the stopper also has an engagement portion that is able to engage with the needle cage of retractable needle apparatus 2105.

FIG. 21E illustrates the engagement component of the stopper of the second barrel with the needle cage component of retractable needle apparatus. Further, after the stopper has combined with the needle cage component of retractable needle apparatus, and after the plunger seal has been combined with the stopper, the needle case-needle combination is able to be retracted. Accordingly, FIG. 21F illustrates retraction of the needle case-needle combination via the retraction of the plunger seal having an engagement portion that is coupled to the stopper which is coupled to the needle case.

While there are a large number of advantages to producing two-component drug delivery systems, the processes that are used to store medicament components may vary. For example, the process for filling a liquid component may be relatively simple, while the process for lyophilizing a lyo component may include the use of a vacuum so as to remove sufficient moisture from a sample. In particular, a lyo may be produced in a process called “freeze-dry” where a liquid is placed in a container and then frozen. The frozen liquid is then dried under a vacuum at an appropriate temperature, such as less than 30° C. Alternatively, liquid drug filling may use a vacuum to draw liquid into a storage barrel. Accordingly, embodiments are provided for having separable drug delivery devices such that each separable component, or “casing,” of the drug delivery device may be filled before combining the casings of a drug delivery device. In this way, a first casing that holds a liquid component may be filled at a first location while a second casing that holds a lyo component may be filled and lyophilized at a second location. In this way, the casings may be filled simultaneously under different conditions since each casing is filled independently of the other and goes through different processes.

Additionally, medicament components that are stored within these separable device casings may be hermetically sealed. This is in contrast to some previous embodiments, such as the device 100 in FIG. 1, where the lyo component does not have an internal seal. Rather, in previous embodiments, the solid components were sealed from the outside environment using an external seal, such as a bag that would seal the entire drug delivery device from outside contaminants. However, this type of external seal would still expose the solid component to air within the sealed bag itself. Further, exposure of the solid component to air external to the drug delivery device would potentially decrease the amount of time that the solid component could be stored.

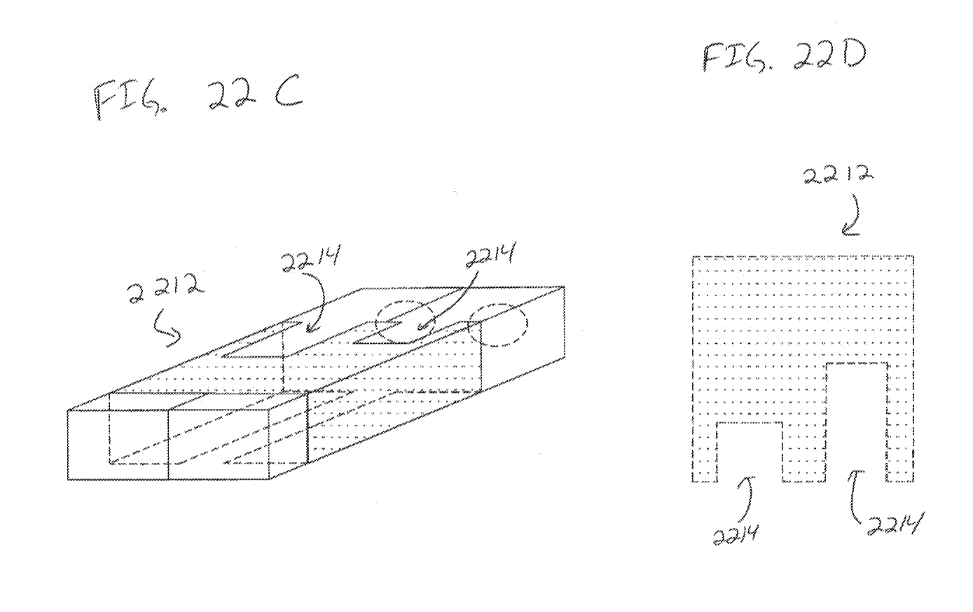

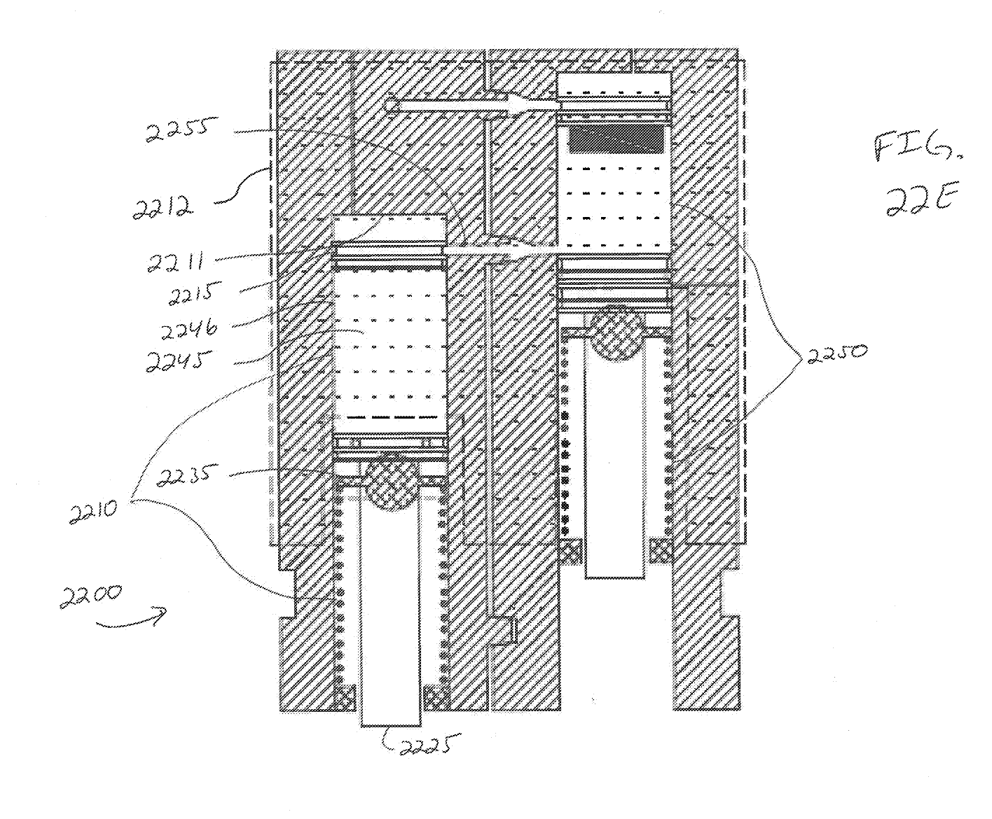

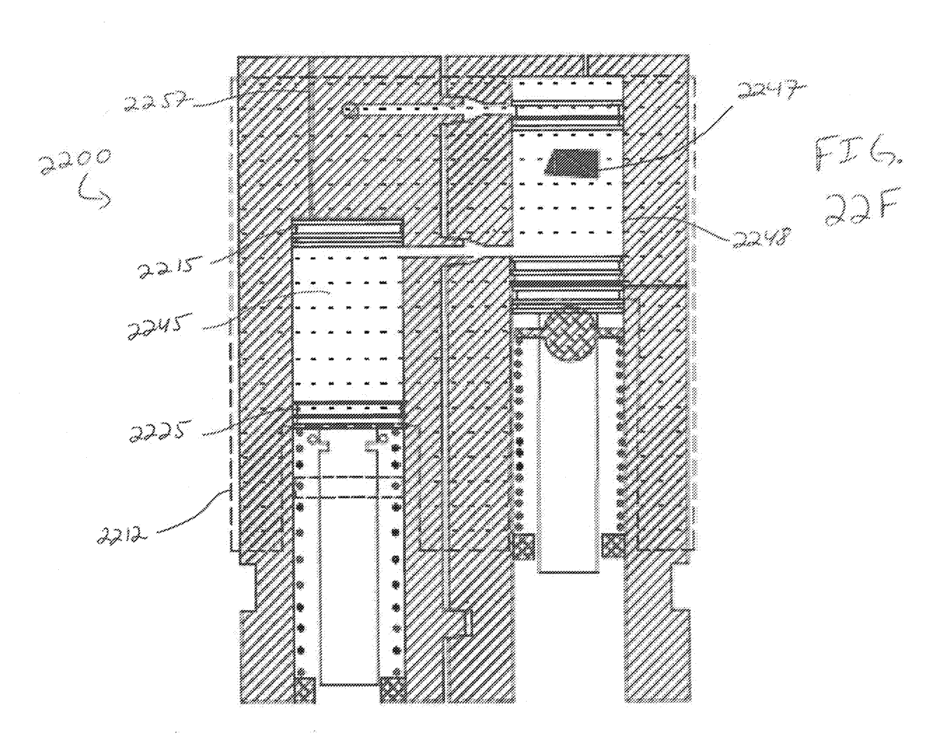

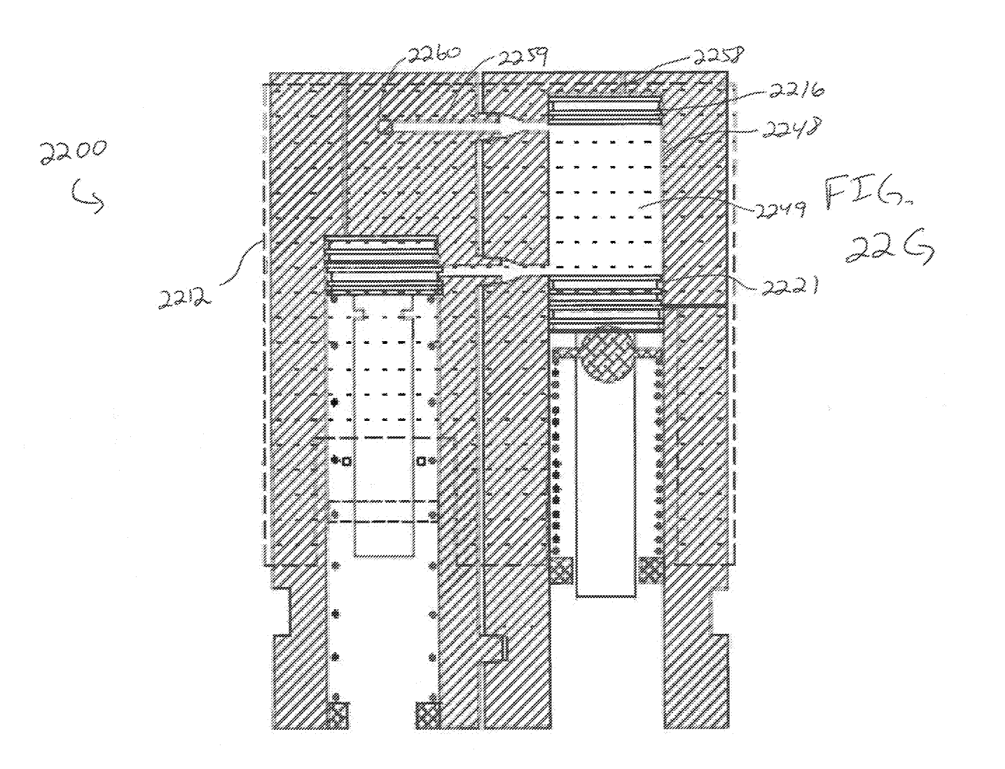

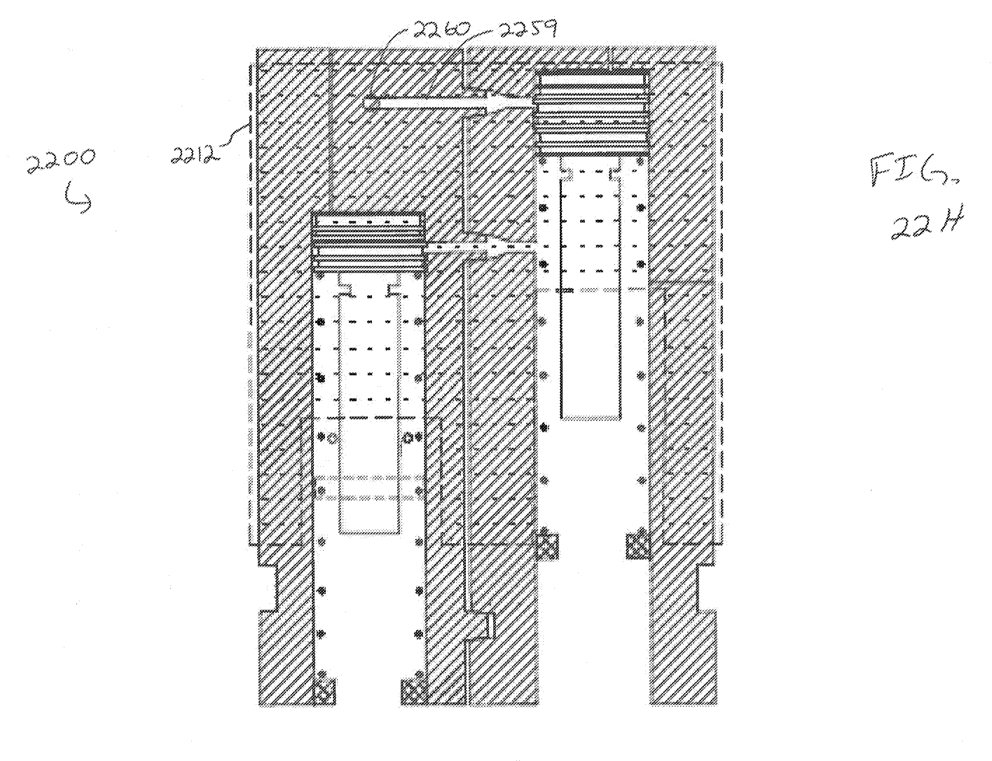

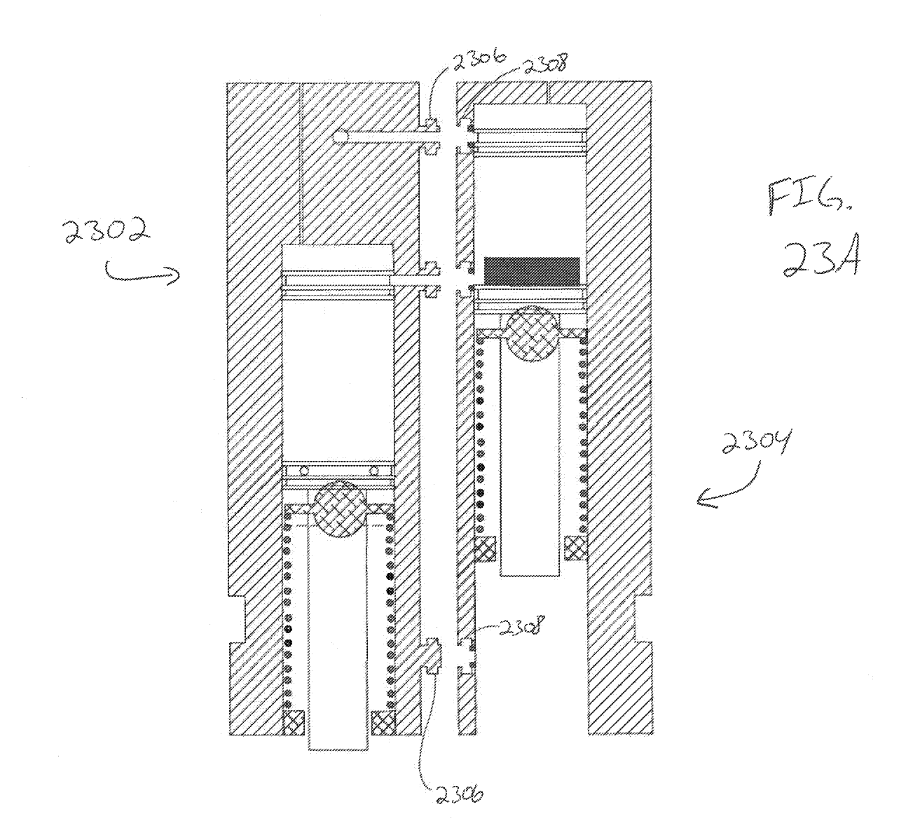

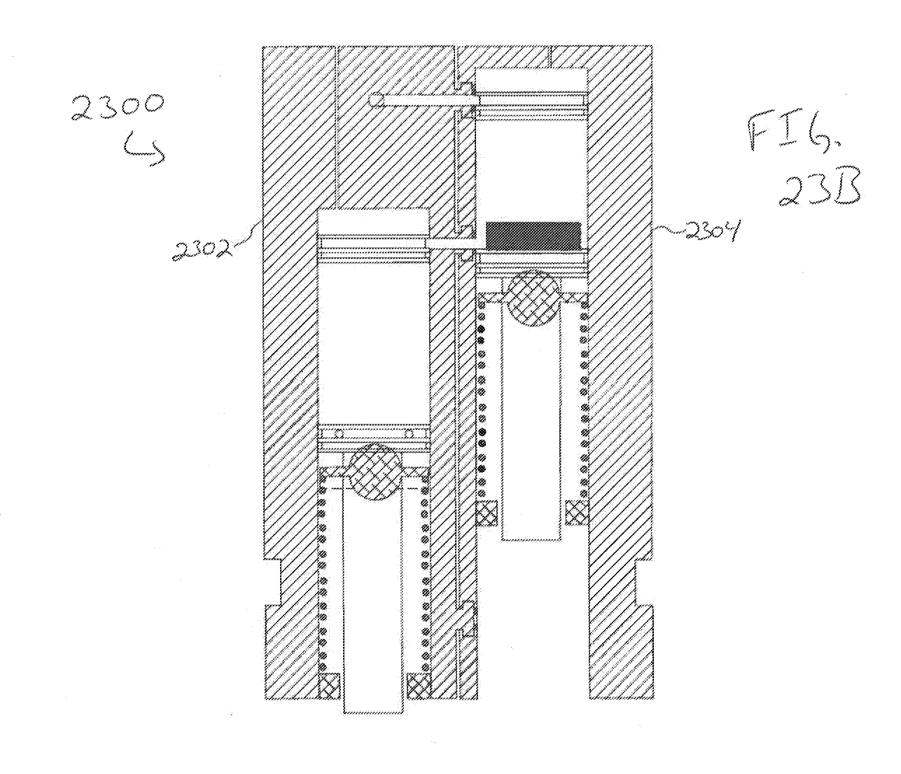

Referring next to FIG. 22, yet another embodiment of a drug delivery device will be described. This embodiment features separable casings of the drug delivery device as well as the hermetic sealing of lyo components within a drug delivery device. Accordingly, a drug delivery device that contains separable, internally sealed chambers is provided in FIGS. 22A-22H in accordance with embodiment 2200. Device 2200 has two casings 2202, 2204 that are able to be coupled. Each casing 2202, 2204 has an internally sealed chamber. The internally sealed chambers may be hermetically sealed, thereby increasing the storage life of drug components stored within the chambers. Once the casings 2202, 2204 are coupled together, the mechanism of drug delivery of embodiment 2200 is similar to that previously discussed with respect to embodiment 100. However, rather than having only one chamber (liquid chamber 145 of embodiment 100) sealed prior to dispensing a stored drug component, two chambers of device 2200 are sealed. As seen in embodiment 2200, a liquid chamber 2246 that contains a liquid component 2245 is formed between stopper 2215 and plunger seal 2220. Additionally, a mixing chamber 2248 that contains a solid component is formed between stopper 2216 and plunger seal 2221.

FIG. 22A illustrates first casing 2202 uncoupled from second casing 2204 of device 2200. As seen in FIG. 22A, first casing 2202 has connector prongs 2206 and second casing 2204 has connector ports 2208. The connector prongs 2206 are shaped to fit into connector ports 2208. Using connector prongs 2206 and connector ports 2208, first casing 2202 may be fit within second casing 2204. For example, first casing 2202 may be connected to second casing 2204 through the use of seals (such as o-rings) or adhesives or press-fit.

When first casing 2202 is coupled with second casing 2204, it forms device 2200 as seen in FIG. 22B. Further, once first casing 2202 is coupled with second casing 2204, a sleeve 2212 may be placed over device 2200 so as to ensure that first casing 2202 continues to be connected to second casing 2204. Sleeve 2212 may press against device 2200 as formed from first casing 2202 connected to second casing 2204. In this way, sleeve 2212 may hold together the casings 2202, 2004 of device 2200. In alternative embodiments, the securing mechanisms between first casing 2202 and second casing 2204 may be sufficient to not require the use of a sleeve to secure the device 2200.

FIGS. 22C and 22D illustrate sleeve 2212 in a perspective view and a front view, respectively. As seen in FIGS. 22C and 22D, sleeve 2212 fits over a device. While the upper part of sleeve 2212 (as oriented with respect to the openings for the plungers) is solid in this embodiment, alternative embodiments may provide a window in sleeve 2212 so that a user may see what is going on inside device 2200. Alternatively, sleeve 2212 may be made of a transparent material such that even when the upper part of sleeve 2212 is solid, the user is still able to see what is going on inside device 2200. Additionally, sleeve 2212 has openings 2214 that allow movement of spring guard caps. An illustration of sleeve 2212 as positioned over 2200 is seen in FIG. 22E.

Once device 2200 has been assembled, the medicament components may be mixed according to similar methods discussed above. As seen in FIGS. 22E-22H, when the plunger 2225 is in a first (loaded) position as illustrated in FIG. 22E, the spring guard cap 2235 prevents the plunger 2225 from moving distally and the stopper 2215 is positioned such that it seals communication channel 2255 thereby preventing the liquid component 2245 from leaking into the communication channel 2255 that leads to mixing chamber 2248 in the second barrel 2250. When spring guard cap 2235 is removed, the compressed spring 2230 is released such that the spring pushes plunger 2225 and plunger seal 2220 distally within the first barrel 2210. The liquid 2245 is typically substantially incompressible and therefore pushes stopper 2215 distally as well to a position where communication channel 2255 is opened, allowing fluid communication between liquid storage chamber 2246 and the second barrel 2250.

In the illustrated embodiment, communication channel 2255 is proximally far enough from the distal end 2211 of first barrel 2210 so as to allow stopper 2215 to move past communication channel 2255 and clear the opening of communication channel 2255, thereby allowing liquid component 2245 to flow into communication channel 2255. This second position is diagrammatically illustrated in FIG. 22F which shows the first plunger 2225 part way through its stroke with the stopper 2215 moved to open the communication channel 2255. In this second position, liquid component 2245 is able to pass from first barrel 2210 through communication channel 2255 to mixing chamber 2248. The air that previously resided in the space between stopper 2215 and the top of first barrel 2210 is vented through vent 2257.

When liquid component 2245 passes through communication channel 2255 and into the mixing chamber 2248, liquid component 2245 interacts with solid component 2247 (which in this example takes the form of a lyo). In particular, lyo component 2247 dissolves within liquid component 2245. This is diagrammatically illustrated in FIG. 22F which shows the first plunger 2225 part way through its stroke. At this point, some (but not all) of the liquid component 2245 has been transferred to the mixing chamber 2248 and has begun dissolving lyo 2247 as diagrammatically illustrated.

Once lyo 2247 has been dissolved in liquid 2245 to form medicament 2249, stopper 2216 may be displaced by the amount of liquid that has passed from liquid chamber 2246 of the first barrel 2210 as illustrated in FIG. 22G. Once the liquid component 2245 has been emptied from the liquid chamber 2246 and passed through to the second barrel 2250, and since the liquid component 2245 is substantially incompressible, the pressure on stopper 2216 is sufficient to have stopper 2216 move distally across second barrel 2250 and over outlet channel 2259. As stopper 2216 is moving across second barrel 2250, the air that occupied the space between stopper 2216 and the end of second barrel 2250 is released via a vent 2258.

With outlet channel 2259 now open, medicament 2249 is able to pass from mixing chamber 2248 through outlet channel 2259 to outlet 2260. As the plunger 2226 and plunger seal 2221 continue movement distally within the second barrel 2250, the mixture 2249 follows the path of least resistance and is thereby forced through outlet 2260 to the patient until the plunger 2226 comes to rest and/or the plunger seal 2221 seals off outlet channel 2259 as illustrated in FIG. 22H.

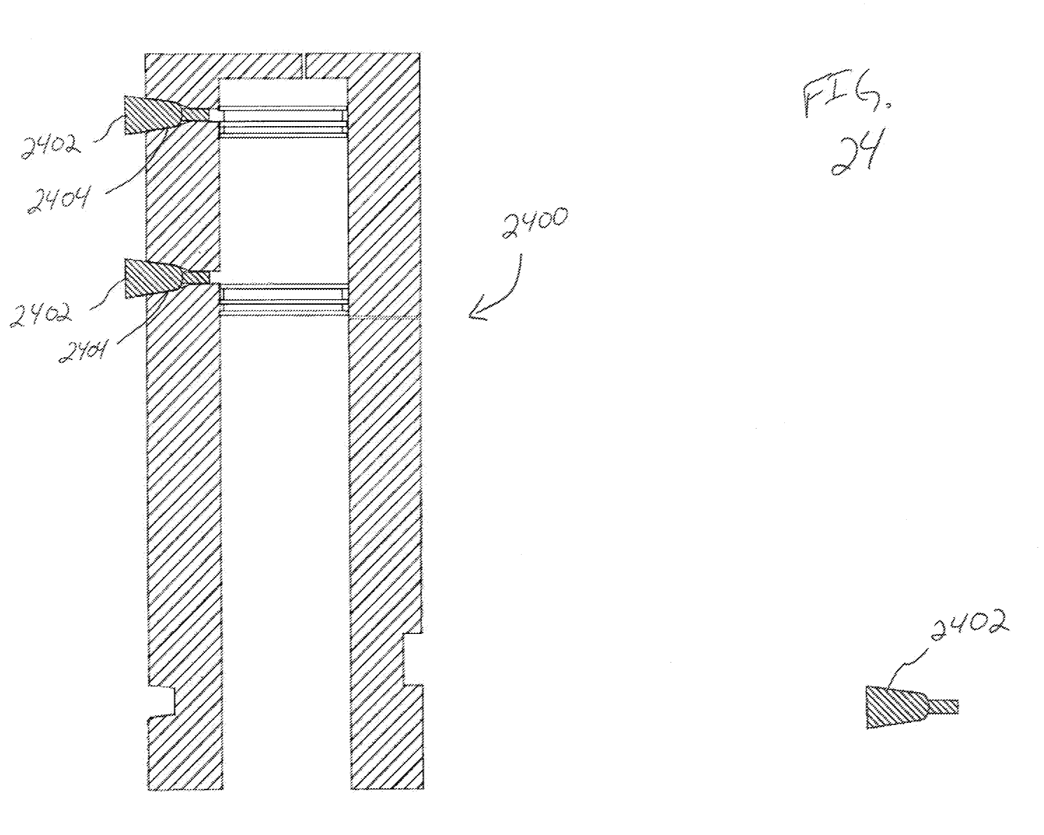

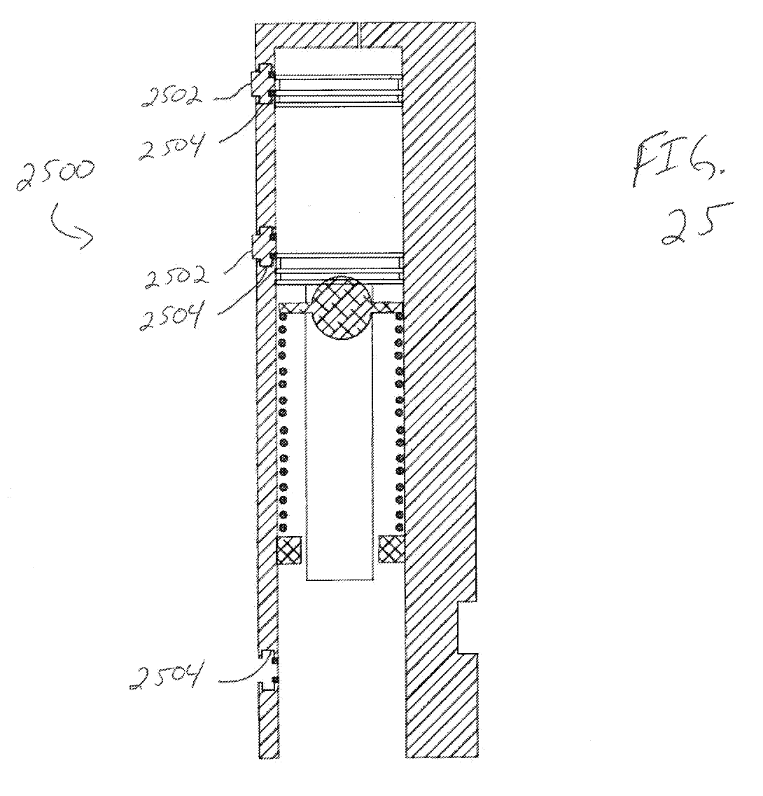

While FIGS. 22A-22H illustrate one example of joining casings to form a device, there are additional ways that two device casings may be joined. For example, device casings may be joined together through the use of ultrasonic, laser bonding, glue, a mechanical lock, or other mechanisms. As another example of device casings that may be coupled, FIGS. 23A and 23B illustrate a drug delivery device with separable, t-shaped casings in accordance with embodiment 2300.

Device 2300 comprises a first casing 2302 and a second casing 2304. FIG. 23A illustrates first casing 2302 uncoupled from second casing 2304 of device 2300. As seen in FIG. 23A, first casing 2302 has connector prongs 2306 that are t-shaped. The connector ports 2308 have complementary t-shaped gaps so that fit within connector ports 2308 of second casing 2304. Connector prongs 2306 may be fit securely within connector ports 2308, such as using a press-fit. Additionally or alternatively, the connector prongs 2306 may be secured within the connector ports 2308 using an adhesive. When first casing 2302 is coupled with second casing 2304, it forms device 2300 as seen in FIG. 23B.

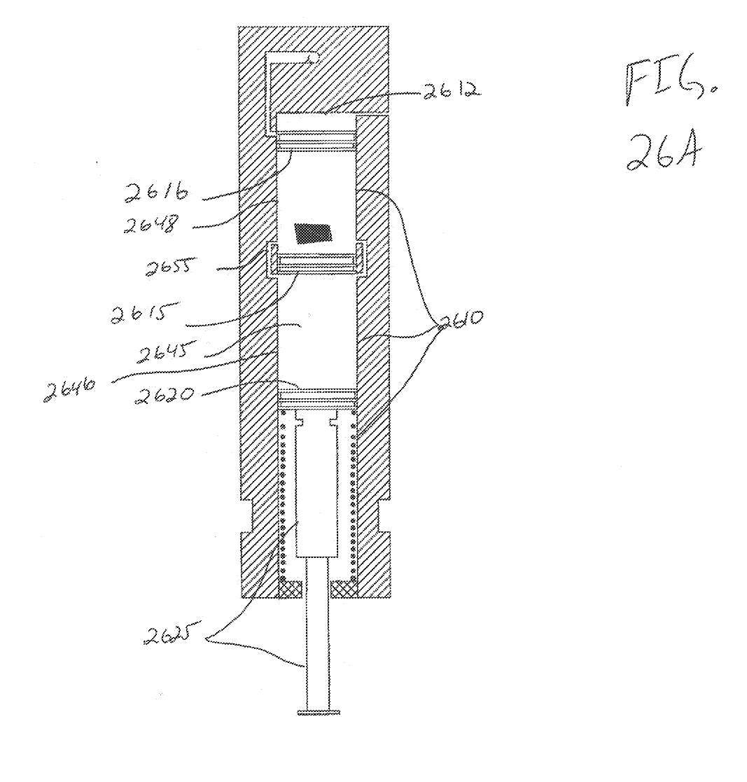

FIGS. 24 and 25 illustrate medicament component holders having angled and mechanical connectors, respectively. FIG. 24 illustrates medicament component holder 2400 having angular connector plugs 2402 and recesses 2404. Angular plugs 2402 fit within recesses 2404 so that plugs 2402 form a tight seal for a component stored within medicament component holder 2400. In practice, medicament component holder 2400 may be sent out to a pharmaceutical company in a form shown in FIG. 24, namely without springs or other components of a device casing. These additional components of a drug delivery device may be added at a later time, and FIG. 25 illustrates a casing having these additional components. As shown in FIG. 24, medicament component holder 2400 may be sent with minimal additional casings needed to load and store a medicament component. For example, with regard to loading holder 2400 with a lyo, a medicament component holder 2400 may be sent to a company that fills a lyo chamber, as bounded by a plunger seal and a stopper, with a lyo solution. The lyo solution comprises a lyo that is in solution with a diluent. Once the lyo solution is dried such that only an appropriate amount of moisture is retained, the vial containing the dried lyo is sealed with a sealing material, such as rubber. The appropriate among of moisture that is retained may vary based on each lyo medicament. The stored lyo may be transported in the medicament component holder 2400, as sealed by plunger seal, stopper, and connector prongs 2402.

Similarly, FIG. 25 illustrates medicament component holder 2500 having t-shaped connector plugs 2502 and recesses 2504. The t-shaped plugs 2502 may be placed in recesses 2504 by vertically aligning the t-shaped components and then setting the t-shaped plugs 2502 into the recesses 2504. There may also be additional forms of adhesion that are used to secure t-shaped plugs 2502. Plugs 2502 fit within t-shaped connector recesses 2504 so that plugs 2502 form a tight seal for a component stored within device casing 2500. Holder 2500 is similar to holder 2400 except that holder 2500 may also include spring activation components. In this way, holder 2500 may be sealed and/or ready to be combined with another casing component. When holder 2500 is ready to be combined with another casing component, plugs 2502 may be removed and replaced with t-shaped connector prongs of the other casing component.

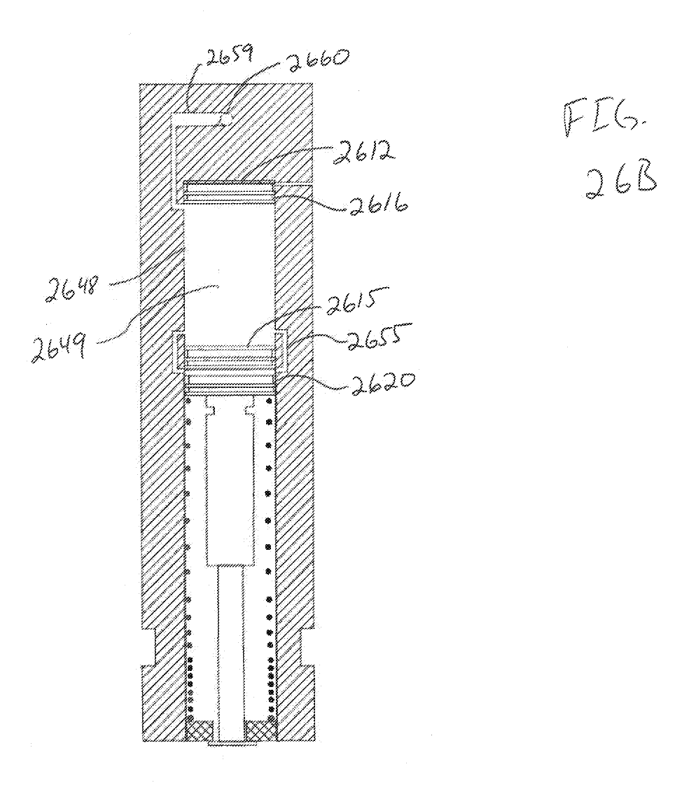

Another example of a drug delivery device with sealed chambers is seen in FIGS. 26A-26B. Similar to FIGS. 7A-7D above, a liquid component 2645 passes through side channels 2655 to dissolve a lyo component in a mixing chamber 2648. Since the volume of liquid is greater than the amount of space available to hold the liquid in the mixing chamber 2648 but for the displacement of stopper 2616, the liquid is able to displace 2616 to the end wall 2612 of the device. FIG. 26A illustrates plunger seal 2620 as it begins to move distally across barrel 2610, allowing the liquid component 2645 to pass through the side channels 2655. Once the entire liquid component 2645 has passed through, the plunger seal 2620 is pressed distally far enough to seal off a portion of the side channels 2655 and press against stopper 2615. Additionally, since the liquid component 2645 is generally incompressible, the movement of the plunger seal 2620 also indirectly moves stopper 2616 by transferring a sufficient amount of the liquid to displace stopper 2616 which previously sealed the mixing chamber 2648 from the outlet channel. In the illustrated embodiment of FIG. 26B, outlet channel 2659 is proximally far enough from the distal end 2612 of barrel 2610 so as to allow stopper 2616 to move past outlet channel 2659 and clear the opening of outlet channel 2659, thereby allowing medicament 2649 to flow into outlet channel 2659.

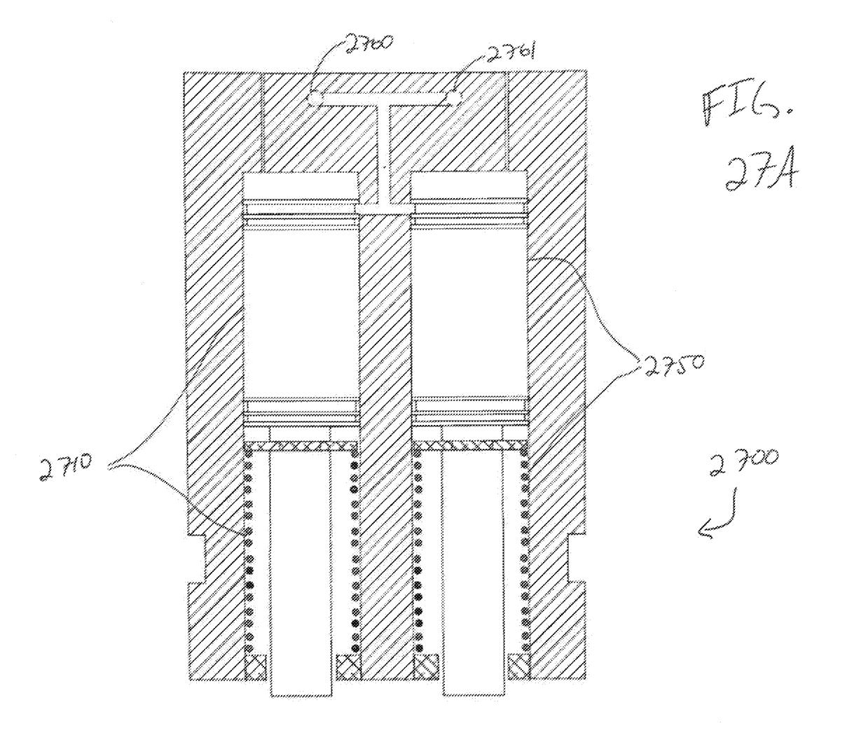

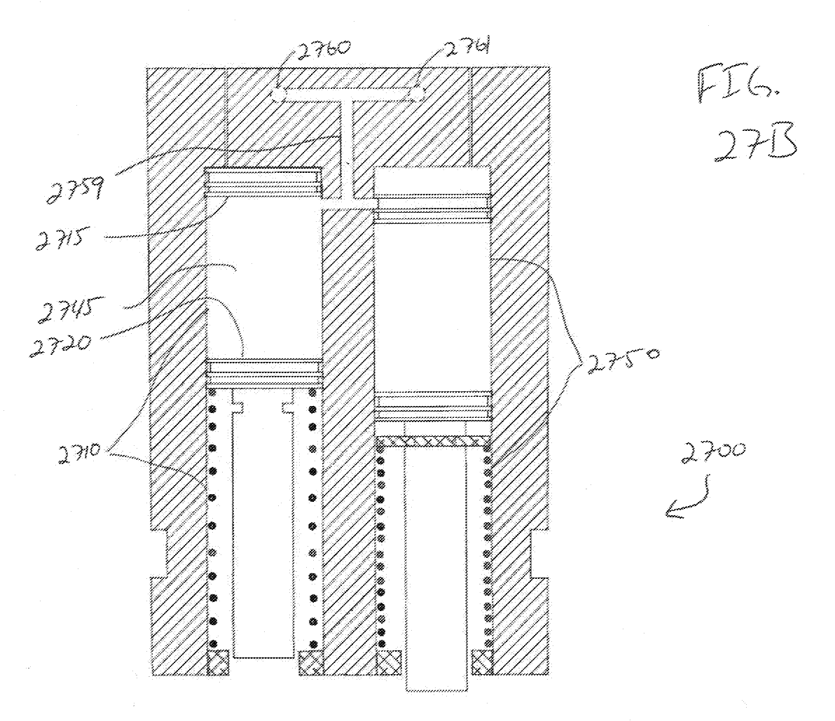

While previously described embodiments provide one outlet through which medicament mixtures can pass through to be injected into a patient, additional embodiments provide that multiple outlets may be used. For example, FIGS. 27A-27C illustrate a drug delivery device that has two outlets. The drug delivery device of FIGS. 27A-27C is in accordance with an embodiment 2700. As seen in FIG. 27A, device 2700 has outlets 2760, 2761. Each outlet 2760, 2761 is able to provide medicament to a patient through microneedles as discussed above, and a drug delivery device may use a number of microneedles to help distribute a medicament across a larger surface area of a patient's skin. While the current embodiment illustrates the use of two outlets, further embodiments may include more outlets, such as up to sixteen outlets connected to a corresponding set of 16 microneedles.

Additionally, device 2700 has two liquid chambers that are each hermetically sealed. First barrel 2710 has a liquid chamber that is formed between plunger seal 2720 and stopper 2715, while second barrel 2750 also has a liquid chamber that is formed between a plunger seal and a stopper. Each barrel 2710, 2750 has enough room at the distal end of the barrels to have stoppers 2715, 2716 pass over outlet channel 2759, allowing each liquid component to pass from its respective liquid chamber and through to outlets 2760, 2761. The movement of liquid component 2745 through first barrel 2710 to outlets 2760, 2761 is illustrated in FIGS. 27B-27C. When spring guard cap is removed, as seen in FIG. 27B, the compressed spring is released such that the spring pushes the plunger and plunger seal 2720 distally within the first barrel 2710. The liquid 2745 is typically substantially incompressible and therefore pushes stopper 2715 distally as well to a position where outlet channel 2759 is opened. As seen in FIG. 27C, the plunger may then move distally until plunger seal 2720 comes into contact with stopper 2715, at which point liquid component 2745 has passed through outlet channel 2759 to outlets 2760, 2761. In some embodiments, a patient may choose to use a two-barrel device 2700 to administer one dosage of medicine at a first time (say, in the morning) and another dosage of medicine at a second time (say, in the evening). In this way, the patient may only have to insert the microneedle-based transdermal drug delivery device once for a day's worth of medication.

Although FIGS. 27A-27C only illustrate the activation of barrel 2710, it is also possible for both barrels 2710, 2750 to dispense a component at the same time. Further, when dispensed simultaneously, liquid dispensed from barrels 2710, 2750 may mix as the medicament is being dispensed. Additionally, while two outlets 2760, 2761 are shown in FIGS. 27A-27C, additional outlets may also be provided to increase the surface area over which a medicament is provided to a patient.

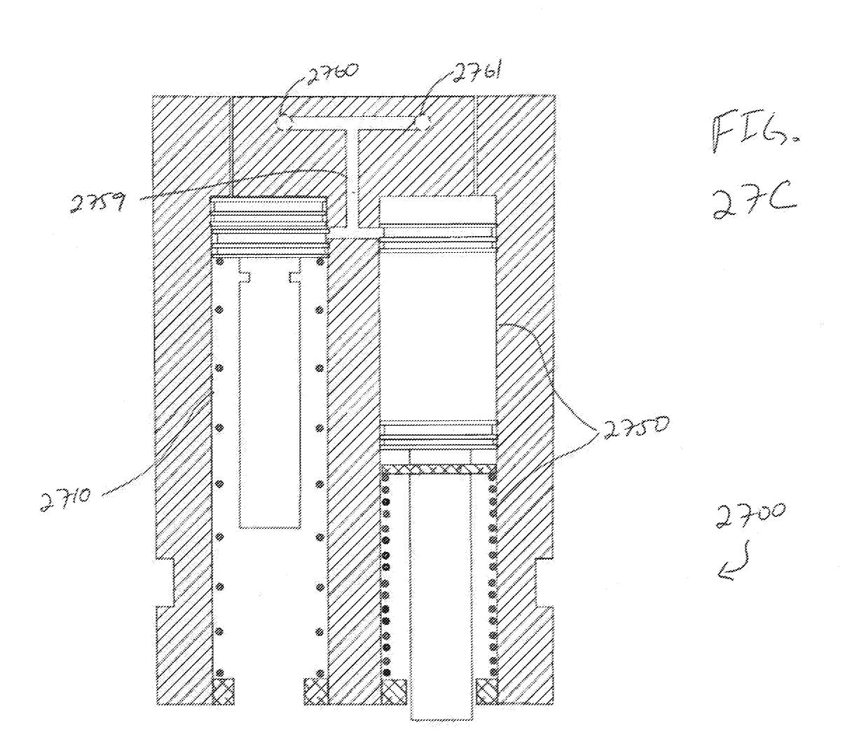

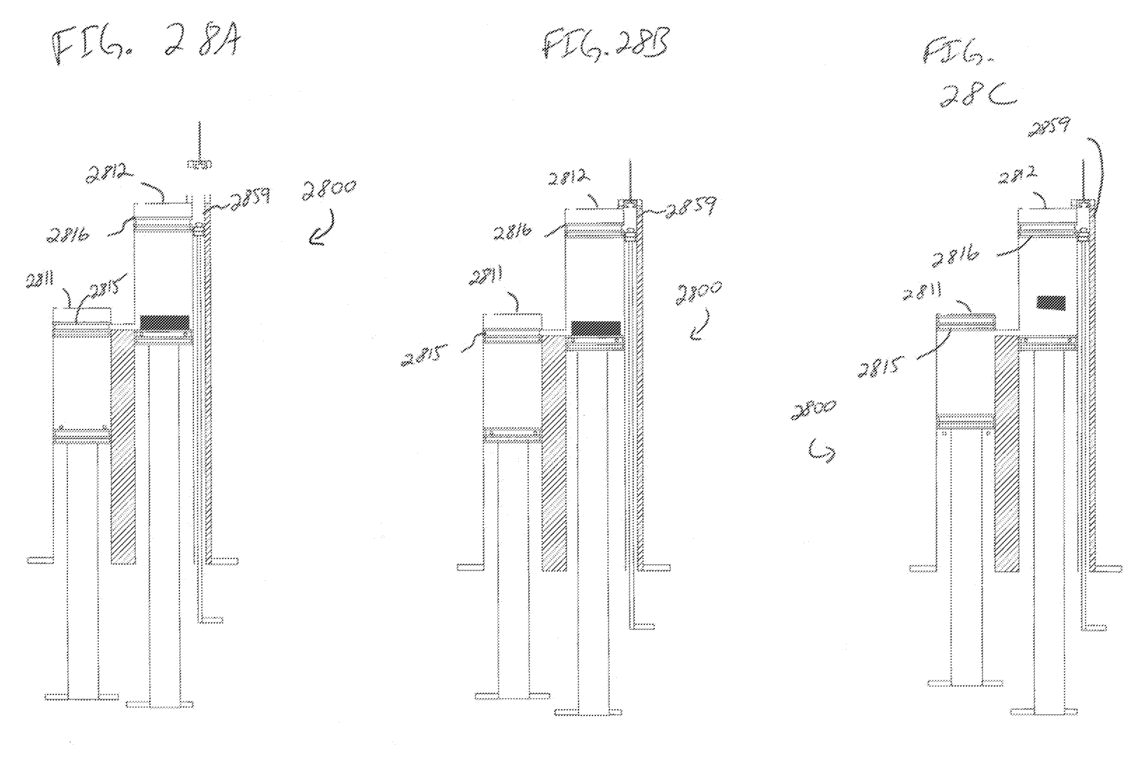

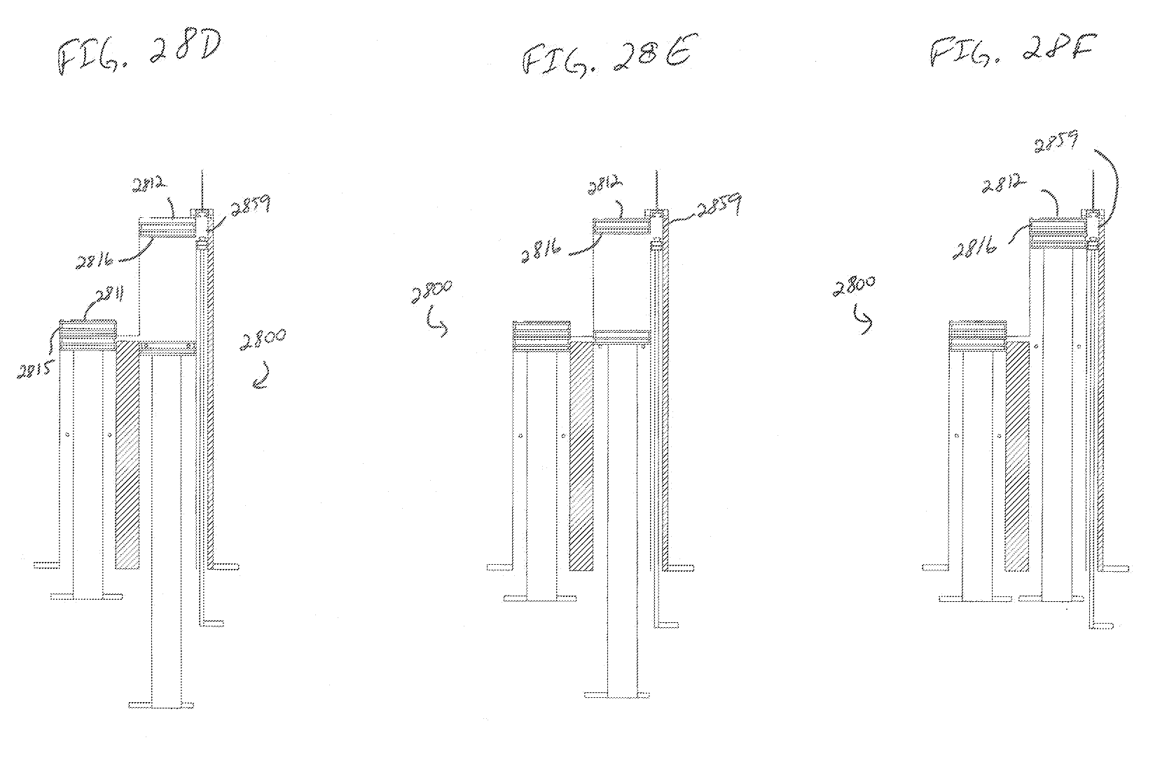

The additional of a second stopper so as to seal a mixing chamber from communicating with an outlet channel may also be used in embodiments relating to removable needles. For example, device 2800 of FIGS. 28A-28F has a second barrel that is similar to the barrel in FIGS. 18A-18F, except the second barrel of device 2800 has a stopper 2816 that seals off the mixing chamber from the outlet channel 2859. In this way, the mixing chamber of device 2800 may be hermetically sealed by adding distal stopper 2816. The process may then proceed with a liquid component passing from a first chamber of 2800 to the second chamber of 2800. In particular, a stopper 2815 within the first chamber may be pressed against the distal wall 2811 of the first barrel, allowing the liquid component to pass through to the second barrel in a manner similar to embodiments discussed above. Once the liquid component has been transferred to the second barrel, however, the liquid displaces the stopper 2816 due to the volume of liquid being greater than the amount of space available to place the liquid in the mixing chamber but for the displacement of 2816 to the end wall 2812 of the second barrel. The displacement is seen in FIG. 28D, where the substantially incompressible liquid from the first barrel has displaced stopper 2816 after it has been transferred to the second barrel. After this has occurred, the outlet channel 2859 is open to have the medicament mixture to pass out of the drug delivery device 2800. The medicament mixture may then be provided to a patient via outlet channel 2859 and the syringe needle as an outlet, as seen in FIGS. 28E and 28F.

Although a number of different features and embodiments have been described, including retracting needles, hermetically sealed chambers, and filling ports, it should be appreciated that the inventions disclosed herein may be implemented in many other forms without departing from the spirit or scope of the invention. For example, retractable needle apparatuses such as those disclosed herein may be compatible with many other forms of syringe-based devices.

Additionally, in drug delivery device embodiments with separable casings, it is possible to individually seal casings of a device that may then be combined at a later date. For example, a liquid component of a medicament may be sealed in a first portion of a device, while a solid component of a medicament may be sealed in a second portion of the device. The first portion of the device may then be coupled with the second portion of the device at a number of different points prior to the administration of the medicament to the patient. For example, the two portions of the device may be coupled at a pharmaceutical manufacturer, they may be combined at a contract research organization, they may be combined by a nurse or other medical professional, or they may be combined by a patient. If the two portions of the device are combined by a person who is unfamiliar with the device, assistance aid may be used to ensure the device is joined appropriately. In this way, the number of possible combinations of medicament components may be increased.

Therefore, the present embodiments should be considered illustrative and not restrictive and the invention is not to be limited to the details given herein, but may be modified within the scope and equivalents of the appended claims.

While this invention has been described in terms of several preferred embodiments, there are alterations, permutations, and various substitute equivalents, which fall within the scope of this invention. For example, outlets as discussed herein may feed a needles or micro-needles that are either open- or closed-bottomed. In particular, the use of closed-bottomed needles or micro-needles may help prevent clogging the hollow interior of the needle/micro-needle with skin debris when then needle/micro-needle is inserted into the skin. Further, a needle/micro-needle as used herein may have at least one side opening communicating with the hollow interior of the needle/micro-needle (e.g., similar to the side openings as described in the incorporated patent application).

Additionally, in embodiments with separable casings, it is possible to individually seal casings of a device that may then be combined at a later date. For example, a liquid component of a medicament may be sealed in a first portion of a device, while a solid component of a medicament may be sealed in a second portion of the device. The first portion of the device may then be coupled with the second portion of the device at a number of different points prior to the administration of the medicament to the patient. For example, the two portions of the device may be coupled at a pharmaceutical manufacturer, they may be combined at a contract research organization, they may be combined by a nurse or other medical professional, or they may be combined by a patient. If the two portions of the device are combined by a person who is unfamiliar with the device, assistance aid may be used to ensure the device is joined appropriately. In this way, the number of possible combinations of medicament components may be increased.

What is claimed is:

1. A method for combining a first liquid component with a second lyo component in a drug mixing apparatus, the method, comprising:
displacing a seal that prevents the first liquid component within a first barrel from coming into contact with the second lyo/liquid component within a second barrel, wherein the first barrel is connected to the second barrel through at least one communication channel;
using an activating mechanism to move the first liquid component from the first barrel through the at least one communication channel to an area within the second barrel holding the second lyo component, allowing the first liquid component to mix with the second lyo component to form a medicament;
displacing a second seal that prevents a medicament from entering an outlet channel; and
using the activating mechanism to eject the mixed medicament from the drug mixing apparatus through the outlet channel.

2. The method of claim 1, wherein the activating mechanism is a plunger.

3. An apparatus for combining a liquid component with a lyo component, the apparatus comprising:
a first barrel containing a liquid between a first plunger seal and stopper when a first plunger of the first barrel is in a first position;
a connecting portion stemming from the first barrel, the connecting portion placed above the stopper when the first plunger is in a first position and below the stopper when the first plunger is in a second position;
a second barrel communicatively connected to a portion within the first barrel that accesses the liquid component when the first plunger is in the second position, the first barrel connected to the second barrel via the connection portion, the second barrel containing a lyo component, wherein the lyo component is placed above a second plunger seal and below a hermetic seal;
an outlet channel that connects the second barrel to an outlet, the outlet channel sealed from the second barrel when the first plunger is in the first position and not sealed when the first plunger is in the second position; and
a second plunger connected to the second plunger seal, the second plunger movable to eject a mixed liquid component and lyo component from the apparatus via an outlet connected to the second chamber via the outlet channel.

4. The apparatus of claim 3, wherein each stopper forms a hermetic seal when the first barrel is in a first position.

5. The apparatus of claim 3, wherein the first barrel is within a first portion of the apparatus and the second barrel is within a second portion of the apparatus, wherein the first portion is separable from the second portion.
